SEQUENCE LISTING (47 sequences)

SEQ ID NO: 1 - DNA, 24 bp, Artificial Sequence
  synthetic construct; forward primer for hsMPC1 exon 1
ccagccccagccgttttacggcag

SEQ ID NO: 2 - DNA, 23 bp, Artificial Sequence
  synthetic construct; reverse primer for hsMPC1 exon 1
ctgaaaggcgcccactgtcaccg

SEQ ID NO: 3 - DNA, 23 bp, Artificial Sequence
  synthetic construct; forward primer for hsMPC1 exon 2
ggcacaaaccacccatgcccagc

SEQ ID NO: 4 - DNA, 25 bp, Artificial Sequence
  synthetic construct; reverse primer for hsMPC1 exon 2
gctgtcacagagctcgtgtctaggc

SEQ ID NO: 5 - DNA, 27 bp, Artificial Sequence
  synthetic construct; forward primer for hsMPC1 exon 3
gtgtctggggtcctgggcattgatttc

SEQ ID NO: 6 - DNA, 31 bp, Artificial Sequence
  synthetic construct; reverse primer for hsMPC1 exon 3
gcaagcaggagctctactatgttgaaagtcc

SEQ ID NO: 7 - DNA, 29 bp, Artificial Sequence
  synthetic construct; forward primer for hsMPC1 exon 4
cagcctactcttgctcggaaatatgttcc

SEQ ID NO: 8 - DNA, 21 bp, Artificial Sequence
  synthetic construct; reverse primer for hsMPC1 exon 4
ccagtcccgcagcactccctc

SEQ ID NO: 9 - DNA, 20 bp, Artificial Sequence
  synthetic construct; forward primer for hsMPC1 cDNA
gccggggtgtcattggctct

SEQ ID NO: 10 - DNA, 23 bp, Artificial Sequence
  synthetic construct; reverse primer for hsMPC1 cDNA
gtgactcagcagcagctggcaat

SEQ ID NO: 11 - DNA, 30 bp, Artificial Sequence
  synthetic construct; forward primer for hsMPC1 ORF
gatcgcggccgcaccatggcgggcgcgttg

SEQ ID NO: 12 - DNA, 37 bp, Artificial Sequence
  synthetic construct; reverse primer for hsMPC1 ORF
gatcggatccttatgcagatgccgttttagtcatctc

SEQ ID NO: 13 - DNA, 19 bp, Artificial Sequence
  synthetic construct; forward primer for mmMPC1 qPCR
gcacggccatggctggagc

SEQ ID NO: 14 - DNA, 24 bp, Artificial Sequence
  synthetic construct; reverse primer for mmMPC1 qPCR
gcaacagagggcgaaagtcatccg

SEQ ID NO: 15 - DNA, 21 bp, Artificial Sequence
  synthetic construct; forward primer for mmMPC2 qPCR
ccgctttacaaccacccggca

SEQ ID NO: 16 - DNA, 24 bp, Artificial Sequence
  synthetic construct; reverse primer for mmMPC2 qPCR
cagcacacaccaatccccatttca

SEQ ID NO: 17 - protein, 109 aa, Homo sapiens
MAGALVRKAADYVRSKDFRDYLMSTHFWGPVANWGLPIAAINDMKKSPEIISGRMTFALCCYSLTFMRFAYKVQPRNWLLFACHATNEVAQLIQGGRLIKHEMTKTASA

SEQ ID NO: 18 - protein, 127 aa, Homo sapiens [sequence omitted]

SEQ ID NO: 19 - protein, 30 aa, Saccharomyces cerevisiae
SPKNYLLFGCHLINETAQLAQGYRFLKYTY

SEQ ID NO: 20 - protein, 30 aa, Drosophila melanogaster
QPRNWLLFACHATNATAQSIQGLRFLHYNY

SEQ ID NO: 21 - protein, 30 aa, Homo sapiens
QPRNWLLFACHATNEVAQLIQGGRLIKHEM

SEQ ID NO: 22 - protein, 30 aa, Caenorhabditis elegans
QPRNLLLFACHFANFSAQGAQLGRFVNHNY

SEQ ID NO: 23 - protein, 30 aa, Danio rerio
QPRNWLLFACHFTNEGAQLIQGSRLIKYNM

SEQ ID NO: 24 - protein, 30 aa, Arabidopsis thaliana
QPRNYLLLACHASNETVQLYQLSRWARAQG

SEQ ID NO: 25 - protein, 97 aa, Saccharomyces cerevisiae
RSFLQKYINKETLKYIFTTHFWGPVSNFGIPIAAIYDLKKDPTLISGPMTFALVTYSGVFMKYALSVSPKNYLLFGCHLINETAQLAQGYRFLKYTY

SEQ ID NO: 26 - protein, 97 aa, Drosophila melanogaster
RRAMSTTASKEWRDYFMSTHFWGPVANWGIPVAALADTQKSPKFISGKMTLALTLYSCIFMRFAYKVQPRNWLLFACHATNATAQSIQGLRFLHYNY

SEQ ID NO: 27 - protein, 97 aa, Homo sapiens
RKAADYVRSKDFRDYLMSTHFWGPVANWGLPIAAINDMKKSPEIISGRMTFALCCYSLTFMRFAYKVQPRNWLLFACHATNEVAQLIQGGRLIKHEM

SEQ ID NO: 28 - protein, 97 aa, Caenorhabditis elegans
TTYFKQHSTAEWKHYFLSTHFWGPVANWGLPLAALGDLKKNPDMISGPMTSALLIYSSVFMRFAWHVQPRNLLLFACHFANFSAQGAQLGRFVNHNY

SEQ ID NO: 29 - protein, 97 aa, Danio rerio
RKAVDHLRSKEFRDYLMSTHFWGPVANWGLPIAAISDMRKSPEIISGRMTFALTCYSLLFMRFAYKVQPRNWLLFACHFTNEGAQLIQGSRLIKYNM

SEQ ID NO: 30 - protein, 95 aa, Arabidopsis thaliana
TSRFQAFLNSPIGPKTTHFWGPIANWGFVAAGLVDMQKPPEMISGNMSSAMCVYSALFMRFAWMVQPRNYLLLACHASNETVQLYQLSRWARAQG

SEQ ID NO: 31 - protein, 95 aa, Saccharomyces cerevisiae
RFAFRRFWQSETGPKTVHFWAPTLKWGLVFAGFSDMKRPVEKISGAQNLSLLSTALIWTRWSFVIKPRNILLASVNSFLCLTAGYQLGRIANYRI

SEQ ID NO: 32 - protein, 95 aa, Saccharomyces cerevisiae
NFAFRRFWNSETGPKTVHFWAPTLKWGLVFAGLNDIKRPVEKVSGAQNLSLLATALIWTRWSFVIKPKNYLLASVNFFLGCTAGYHLTRIANFRI

SEQ ID NO: 33 - protein, 95 aa, Drosophila melanogaster
PPKMRPLWMHPAGPKTIFFWAPIVKWSLVIAGLSDLTRPADKISPNGCLALGATNLIWTRYSLVIIPKNYSLFAVNLFVSLTQLFQLGRYYNYQW

SEQ ID NO: 34 - protein, 95 aa, Homo sapiens
PEKLRPLYNHPAGPRTVFFWAPIMKWGLVCAGLADMARPAEKLSTAQSAVLMATGFIWSRYSLVIIPKNWSLFAVNFFVGAAGASQLFRIWRYNQ

SEQ ID NO: 35 - protein, 95 aa, Caenorhabditis elegans
PAFAKPAWNHAAGPKTVFFWAPTIKWTLIGAGLADLARPADKLSLYQNSALFATGAIWTRYCLVITPINYYLSSVNFFVMCTGLAQLCRIAHYRY

SEQ ID NO: 36 - protein, 95 aa, Danio rerio
PAKLRPFYNHPAGPKTVFFWAPMFKWGLVLAGLADMARPAEKLSTSQSAVLTATGLIWSRYSLVIIPKNWNLFAVNFFVGSAGGSQLYRIWMHNR

SEQ ID NO: 37 - protein, 95 aa, Arabidopsis thaliana
TSKLQAIWNHPAGPKTIHFWAPTFKWGISIANIADFAKPPEKLSYPQQIAVTCTGVIWSRYSMVINPKNWNLFSVNVAMAGTGIYQLARKIKHDY

SEQ ID NO: 38 - DNA, 22 bp, Artificial Sequence
  synthetic construct; forward primer for eGFP
ctcgaggtgagcaagggcgagg

SEQ ID NO: 39 - DNA, 28 bp, Artificial Sequence
  synthetic construct; reverse primer for eGFP
ggtaccttacttgtacagctcgtccatg

SEQ ID NO: 40 - DNA, 29 bp, Artificial Sequence
  synthetic construct; forward primer for dMPC1
cggaattcaactttcggagtgacaacacg

SEQ ID NO: 41 - DNA, 33 bp, Artificial Sequence
  synthetic construct; reverse primer for dMPC1
cgctcgagggctgccgcctgctgctccttagac

SEQ ID NO: 42 - DNA, 21 bp, Artificial Sequence
  synthetic construct; MPC1 siRNA
ucaacuacgagaugaguaatt

SEQ ID NO: 43 - DNA, 21 bp, Artificial Sequence
  synthetic construct; MPC1 si RNA
gggaaaacacagaaugcuatt

SEQ ID NO: 44 - DNA, 21 bp, Artificial Sequence
  synthetic construct; MPC1 siRNA
ccauguaacaaacgaaguatt

SEQ ID NO: 45 - DNA, 21 bp, Artificial Sequence
  synthetic construct; MPC2 siRNA
ccgauaaggugaugcuaaatt

SEQ ID NO: 46 - DNA, 21 bp, Artificial Sequence
  synthetic construct; MPC2 siRNA
uggauaaaguggaguuguutt

SEQ ID NO: 47 - DNA, 21 bp, Artificial Sequence
  synthetic construct; MPC2 siRNA
accaagaacucaaaucuaatt

(19) United States
(12) Patent Application Publication
(10) Pub. No.: US 2015/0099790 A1
(43) Pub. Date: Apr. 9, 2015

(54) COMPOSITIONS AND METHODS FOR MODULATING MITOCHONDRIAL PYRUVATE CARRIER ACTIVITY

(71) Applicant: UNIVERSITY OF UTAH RESEARCH FOUNDATION, Salt Lake City, UT (US)

(72) Inventors: Jared Rutter, Salt Lake City, UT (US); Carl Thummel, Salt Lake City, UT (US); Daniel K. Bricker, Salt Lake City, UT (US); Thomas Orsak, Woodbridge, VA (US)

(21) Appl. No.: 14/400,274

(22) PCT Filed: May 8, 2013
(86) PCT No.: PCT/US13/40220

§ 371 Date: Nov. 10, 2014

Related U.S. Application Data
(60) Provisional application No. 61/645,315, filed on May 10, 2012.
(60) Provisional application No. 61/664,434, filed on Jun. 26, 2012.

(51) Int. Cl. C12N 15/113; C12Q 1/68; A61K 31/405

(52) U.S. Cl. CPC C12N 15/113; A61K 31/405; C12Q 1/6883; C12N 2310/14; C12N 2320/30; C12Q 2600/158
USPC 514/44 A; 514/419; 506/9

(57) ABSTRACT

Disclosed herein are methods and compositions for the diagnosis and treatment of conditions associated with aberrant pyruvate metabolism, and symptoms thereof, in a subject. Disclosed herein are methods of detecting an aberrant pyruvate metabolism-associated condition, and symptoms thereof, in a subject, by the expression level of MPC1 or MPC2. Disclosed herein are methods of detecting an aberrant pyruvate metabolism-associated condition, and symptoms thereof, in a subject, by the activity level of MPC1 or MPC2. Disclosed herein are methods of determining responsiveness to a treatment for an aberrant pyruvate metabolism associated condition, and symptoms thereof, in a subject.

### CROSS-REFERENCE TO RELATED APPLICATIONS

This application claims benefit of U.S. Provisional Patent Application No. 61/645,315 filed May 10, 2012 and the benefit of U.S. Provisional Patent Application No. 61/664,434 filed Jun. 26, 2012, each application of which is hereby incorporated by reference it its entirety.

### STATEMENT REGARDING FEDERALLY SPONSORED RESEARCH OR DEVELOPMENT

This invention was made with government support under Grants RO1GM087346, GM0RC1DK086426, R24DK092784, and T32GM007464 awarded by NIH. The government has certain rights in the invention.

### REFERENCE TO SEQUENCE LISTING

The Sequence Listing submitted on May 8, 2013 as a text file named “21101_{—}0267P1_{—}2013_{—}05_{—}08 Sequence_Listing.txt,” created on May 8, 2013 and having a size of 21,838 bytes is hereby incorporated by reference pursuant to 37 C.F.R. §1.52(e)(5).

### FIELD OF THE INVENTION

The invention relates generally to the diagnosis and treatment of conditions associated with aberrant pyruvate metabolism, and symptoms thereof, in a subject. Specifically, described herein are a) methods of detecting an aberrant pyruvate metabolism associated condition, and symptoms thereof, in a subject, by determining the expression level of MPC1 or MPC2 b) methods of detecting an aberrant pyruvate metabolism associated condition, and symptoms thereof, in a subject, by the determining the activity level of MPC1 or MPC2, and c) methods of determining responsiveness to a treatment for an aberrant pyruvate metabolism associated condition, and symptoms thereof, in a subject.

### BACKGROUND OF THE INVENTION

Mitochondria are organelles that play a central role in all aspects of biology, including energy production, intermediary metabolism, and apoptosis. These broad cellular functions also place mitochondria as a central player in human health. Mitochondrial dysfunction is associated with a wide range of diseases, including cancer, type 2 diabetes, and most neurodegenerative disorders. As a result of these wide-ranging activities, many efforts have focused on identifying and characterizing the mitochondrial proteome, with over 1,000 proteins identified to date in mammals. However, roughly one-quarter of these proteins remain uncharacterized. These include many proteins that are highly conserved throughout eukarya, a strong indication that they perform a fundamentally important function. Prior studies of uncharacterized mitochondrial conserved proteins support this, revealing new roles for these proteins in aspects of mitochondrial function and diseases such as cancer.

Pyruvate occupies a node in the regulation of carbon metabolism as it is the end product of glycolysis and a major substrate for the tricarboxylic acid (TCA) cycle in mitochondria. Pyruvate lies at the intersection of these catabolic pathways with anabolic pathways for lipid synthesis, amino acid biosynthesis, and gluconeogenesis. As a result, the failure to correctly partition carbon between these fates influences the altered metabolism evident in diabetes, obesity and cancer [Hanahan et al., 2011; Kahn et al., 2006]. Due to the importance of pyruvate, what is needed are diagnostic and treatment methods based on the expression of activity of the mitochondrial pyruvate carrier (MPC) genes and proteins. Furthermore, what is needed are methods of screening for MPC modulators.

### BRIEF SUMMARY OF THE INVENTION

Described herein are methods of detecting an aberrant pyruvate metabolism associated condition in a subject comprising determining the expression level of MPC1 or MPC2 in a sample from the subject and comparing the expression level to the expression level of MPC1 or MPC2 in a normal sample.

Disclosed herein is a method of detecting an aberrant pyruvate metabolism associated condition in a subject, the method comprising: (a) isolating nucleic acid from a subject; (b) amplifying the nucleic acid isolated in step (a); (c) determining the expression level of MPC1 or MPC2 present in the amplified nucleic acid of step (b) and (d) comparing the expression level obtained in step (c) to the expression level of MPC1 or MPC2 in a normal sample.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) determining the expression level of MPC1 or MPC2; and (e) comparing the expression level obtained in step (d) to the expression level of MPC1 or MPC2 in a normal sample. In an aspect, a decrease in the expression level of MPC1 or MPC2 obtained in step (d) when compared to the expression level of MPC1 or MPC2 in the normal sample can indicate that the subject has an aberrant pyruvate metabolism associated condition.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) determining the expression level of MPC1; (e) determining the expression level of MPC2; (f) comparing the expression level obtained in step (d) to the expression level of MPC1 in a normal sample; and (g) comparing the expression level obtained in step (e) to the expression level of MPC1 in a normal sample.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) sequencing the cDNA; (e) identifying one or more mutations in the MPC1 sequence, wherein the one or more mutations is T236A or C289T, and wherein the presence of the one or more mutations indicates that the subject has an aberrant pyruvate metabolism associated condition.

Also described herein are methods for of detecting an aberrant pyruvate metabolism associated condition in a subject comprising determining the activity of MPC1 or MPC2 in a sample from the subject and comparing the activity level to the activity level of MPC1 or MPC2 in a normal sample.

Disclosed herein is a method of detecting an aberrant pyruvate metabolism associated condition in a subject, the method comprising: (a) obtaining cells from a subject; (b) isolating protein from the cells; (c) determining the activity level of MPC1 or MPC2; and (d) comparing the activity level obtained in step (c) to the activity level of MPC1 or MPC2 in a normal sample. In an aspect of a disclosed method, a decrease in the activity level of MPC1 or MPC2 obtained in step (c) when compared to the activity level of MPC1 or MPC2 in the normal sample can indicate that the subject has an aberrant pyruvate metabolism associated condition.

Disclosed herein is a method of detecting an aberrant pyruvate metabolism associated condition in a subject, the method comprising: (a) obtaining cells from a subject; (b) isolating protein from the cells; (c) determining the amount of MPC1 or MPC2; and (d) comparing the amount obtained in step (c) to the amount of MPC1 or MPC2 in a normal sample.

Also described herein are methods for detecting an aberrant pyruvate metabolism associated condition in a subject comprising determining in the subject the identity of one or more point mutations in the MPC1 or MPC2 gene wherein the presence of the one or more mutations indicates the presence of an aberrant pyruvate metabolism associated condition in the subject.

Also described herein are methods for detecting an aberrant pyruvate metabolism associated condition in a subject comprising determining in the subject the identity of one or more mutations in the MPC1 protein, wherein the presence of the one or more mutations indicates the presence of an aberrant pyruvate metabolism associated condition in the subject.

Also described herein are methods of treating a subject with an aberrant pyruvate metabolism associated condition, the method comprising administering to the subject in need thereof an effective amount of one or more MPC1/MPC2 complex modulators in an amount sufficient to ameliorate the aberrant pyruvate metabolism associated condition.

Disclosed herein is a method of detecting an aberrant pyruvate metabolism associated condition in a subject, the method comprising: identifying one or more mutations in a MPC1 protein, wherein the one or more mutations is Leu79His or Arg97Trp, and wherein the presence of the one or more mutations indicates that the subject has an aberrant pyruvate metabolism associated condition.

Disclosed herein is a method of detecting an aberrant pyruvate metabolism associated condition in a subject, the method comprising: (a) obtaining cells from a subject; (b) isolating protein from the cells; (c) sequencing the isolated protein; (d) identifying one or more mutations in the MPC1 protein, wherein the one or more mutations is Leu79His or Arg97Trp, and wherein the presence of the one or more mutations indicates that the subject has an aberrant pyruvate metabolism associated condition. In an aspect, a disclosed method can comprise (e) determining the activity level of MPC1 or MPC2; and (f) comparing the activity level obtained in step e to the activity level of MPC1 or MPC2 in a normal sample, wherein a decrease in the activity level of MPC1 or MPC2 obtained in step (e) when compared to the activity level of MPC1 or MPC2 in the normal sample can confirm that the subject has an aberrant pyruvate metabolism associated condition.

Also described herein are methods of ameliorating one or more symptoms associated with an aberrant pyruvate metabolism associated condition comprising administering to a subject in need thereof an effective amount of one or more MPC1/MPC2 complex modulators.

Also described herein are methods for treating a subject with an aberrant pyruvate metabolism associated condition, the method comprising administering to the subject in need thereof an effective amount of a composition comprising MPC1-D118G in an amount sufficient to ameliorate the aberrant pyruvate metabolism associated condition.

Also described herein are methods of ameliorating one or more symptoms associated with an aberrant pyruvate metabolism associated condition comprising administering to a subject in need thereof an effective amount of a composition comprising MPC1-D118G in an amount sufficient to ameliorate the one or more symptoms associated with an aberrant pyruvate metabolism associated condition.

Also described herein are methods of treating cancer by inhibiting or reversing the Warburg effect comprising administering to a subject in need thereof an effective amount of one or more MPC1/MPC2 complex modulators in an amount sufficient to inhibit or reverse the Warburg effect.

Also described herein are methods of treating a subject diagnosed with a mitochondrial pyruvate oxidation defect comprising administering to the subject in need thereof an effective amount of one or more pharmaceutical agents, wherein the one or more pharmaceutical agents restore MPC1 or MPC2 expression or activity in the subject.

Disclosed herein is a method for treating a subject diagnosed with an aberrant pyruvate metabolism associated condition, the method comprising: administering to a subject in need thereof an effective amount of a composition comprising one or more MPC1/MPC2 complex modulators; and ameliorating one or more symptoms associated with the aberrant pyruvate metabolism associated condition.

Disclosed herein is a method for treating a subject diagnosed with an aberrant pyruvate metabolism associated condition, the method comprising: administering to a subject in need thereof an effective amount of a composition comprising MPC1-D118G; and ameliorating one or more symptoms associated with the aberrant pyruvate metabolism associated condition.

Also described herein are methods of determining a subject's responsiveness to a treatment for an aberrant pyruvate metabolism associated condition.

Also described herein are methods of determining a subject's lack of responsiveness to a treatment for an aberrant pyruvate metabolism associated condition.

Also described herein are methods of determining a subject's responsiveness to a treatment for an aberrant pyruvate metabolism associated condition.

Disclosed herein is a method of determining responsiveness to a treatment in a subject diagnosed with an aberrant pyruvate metabolism associated condition, the method comprising: (a) obtaining cells from a subject; (b) isolating RNA from the cells; (c) synthesizing cDNA from the isolated RNA; (d) determining the expression level of MPC1 or MPC2; (e) administering a composition comprising a pharmaceutical agent to the subject; (f) repeating steps (a)-(d); and (g) comparing the expression level MPC1 or MPC2 obtained in step (f) to the expression level of MPC1 or MPC2 obtained in step (d), wherein if the expression level obtained in step (f) is greater than the expression level obtained in step (d), then the subject is responsive to the treatment; wherein if the expression level obtained in step (f) is about equal to the expression level obtained in step (d), then the subject is not responsive to the treatment; and wherein if the expression level obtained in step (f) is less than the expression level obtained in step (d), then the subject is not responsive to the treatment.

Disclosed herein is a method of determining responsiveness to a treatment in a subject diagnosed with an aberrant pyruvate metabolism associated condition, the method comprising: (a) obtaining cells from a subject; (b) isolating protein from the cells; (c) determining the activity level of MPC1 or MPC2; (d) administering a composition comprising a pharmaceutical agent to the subject; (c) repeating steps (a)-(c); and (f) comparing the activity level of MPC1 or MPC2 obtained in step (e) to the activity level of MPC1 or MPC2 obtained in step (c), wherein if the activity level obtained in step (e) is greater than the activity level obtained in step (c), then the subject is responsive to the treatment, wherein if the activity level obtained in step (e) is about equal to the activity level obtained in step (c), then the subject is not responsive to the treatment; and wherein if the activity level obtained in step (e) is less than the activity level obtained in step (c), then the subject is not responsive to the treatment.

Disclosed herein is a method of determining responsiveness to a treatment in a subject diagnosed with an aberrant pyruvate metabolism associated condition, the method comprising: (a) obtaining cells from a subject; (b) isolating protein from the cells; (c) determining the amount of MPC1 or MPC2; (d) administering a composition comprising a pharmaceutical agent to the subject; (e) repeating steps (a)-(c); and (f) comparing the amount of MPC1 or MPC2 obtained in step (e) to the amount of MPC1 or MPC2 obtained in step (c), wherein if the amount obtained in step (e) is greater than the amount obtained in step (c), then the subject is responsive to the treatment, wherein if the amount obtained in step (e) is about equal to the amount obtained in step (c), then the subject is not responsive to the treatment; and wherein if the amount obtained in step (e) is less than the amount obtained in step (c), then the subject is not responsive to the treatment.

Also described herein are methods screening for a pharmaceutical agent effective in treating an aberrant pyruvate metabolism associated condition.

Also described herein are methods of screening for a pharmaceutical agent effective in ameliorating one or more symptoms associated with an aberrant pyruvate metabolism associated condition.

Disclosed herein is a method of screening for a pharmaceutical agent effective in treating an aberrant pyruvate metabolism associated condition, the method comprising (a) determining the expression level of MPC1 or MPC2 in a first sample from a subject; (b) administering a composition comprising a pharmaceutical agent to the subject; (c) determining the expression level of MPC1 or MPC2 in a second sample from the subject; and (d) comparing the expression level of MPC1 or MPC2 obtained in step (c) to the expression level of MPC1 or MPC2 obtained in step (a); wherein if the expression level obtained in step (c) is greater than the expression level obtained in step (a), then the composition comprising the pharmaceutical agent is effective in treating an aberrant pyruvate metabolism associated condition; wherein if the expression level obtained in step c is about equal to the expression level obtained in step (a), then the composition comprising the pharmaceutical agent is not effective in treating an aberrant pyruvate metabolism associated condition; and wherein if the expression level obtained in step (c) is less than the expression level obtained in step (a), then the composition comprising the pharmaceutical agent is not effective in treating an aberrant pyruvate metabolism associated condition.

Disclosed herein is a method of screening for a pharmaceutical agent effective in treating an aberrant pyruvate metabolism associated condition, the method comprising (a) determining the activity level of MPC1 or MPC2 in a first sample from a subject; (b) administering a composition comprising a pharmaceutical agent to the subject; (c) determining the activity level of MPC1 or MPC2 in a second sample from the subject; and (d) comparing the activity level of MPC1 or MPC2 obtained in step (c) to the activity level of MPC1 or MPC2 obtained in step (a), wherein if the activity level of obtained in step (c) is greater than the activity level obtained in step (a), then the composition comprising the pharmaceutical agent is effective in treating an aberrant pyruvate metabolism associated condition; wherein if the activity level obtained in step (c) is about equal to the activity level obtained in step (a), then the composition comprising the pharmaceutical agent is not effective in treating an aberrant pyruvate metabolism associated condition; and wherein if the activity level obtained in step (c) is less than the activity level obtained in step (a), then the composition comprising the pharmaceutical agent is not effective in treating an aberrant pyruvate metabolism associated condition.

Disclosed herein is a method of screening for a pharmaceutical agent effective in treating an aberrant pyruvate metabolism associated condition, the method comprising: (a) determining the amount of MPC1 or MPC2 in a first sample from a subject; (b) administering a composition comprising a pharmaceutical agent to the subject; (c) determining the amount of MPC1 or MPC2 in a second sample from the subject; and (d) comparing the amount of MPC1 or MPC2 obtained in step (c) to the amount of MPC1 or MPC2 obtained in step (a), wherein if the amount of obtained in step c is greater than the activity level obtained in step a, then the composition comprising the pharmaceutical agent is effective in treating an aberrant pyruvate metabolism associated condition; wherein if the amount obtained in step (c) is about equal to the amount obtained in step (a), then the composition comprising the pharmaceutical agent is not effective in treating an aberrant pyruvate metabolism associated condition; and wherein if the amount obtained in step (c) is less than the activity level obtained in step (a), then the composition comprising the pharmaceutical agent is not effective in treating an aberrant pyruvate metabolism associated condition.

Also described herein are methods of screening for a drug-resistant MPC mutant organism.

Disclosed herein is a method of screening for a drug-resistant MPC mutant organism, the method comprising (a) administering to an organism a composition comprising one or more MPC inhibitors; and (b) determining the viability of the organism following the administration of the composition, wherein a viable organism is a drug-resistant MPC mutant organism.

Also described herein are methods of screening for a mutant MPC peptide effective in treating an aberrant pyruvate metabolism associated condition.

Disclosed herein is a method of screening for a mutant MPC peptide effective in treating an aberrant pyruvate metabolism associated condition, the method comprising (a) administering to an organism a composition comprising one or more MPC inhibitors; (b) determining the viability of the organism following the administration of the composition, wherein if the organism is viable, then (i) isolating the MPC1 or MPC2 peptide from the viable organism, and (ii) sequencing the isolated MPC1 or MPC2 peptide, wherein the sequenced MPC peptide is a mutant MPC peptide effective in treating an aberrant pyruvate metabolism associated condition.

Also described herein are methods of screening for a pharmaceutical agent effective in treating a mitochondrial pyruvate oxidation defect.

Also described herein are methods of screening for an MPC agonist or activator.

Disclosed herein is an in vitro method of screening for an MPC modulator, the method comprising: administering to a cell an effective amount of a composition comprising a candidate MPC modulator; and determining the level of pyruvate oxidation in the cell following administration of the composition, wherein an increase in the level of pyruvate oxidation indicates that the candidate MPC modulator is an MPC modulator.

Also described herein are MPC mutant organisms.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows expression of Mpc1 and Mpc2 in mitochondrial inner membranes via Mpc1-GFP and mitochondrial targeted RFP (mtRFP), immunoprecipitations, and immunoblotting of whole cell lysate (WCL) and post mitochondrial supernatant (PMS). FIG. 1 also shows serial dilutions of the indicated yeast strains spotted on synthetic media lacking leucine.

FIG. 2 shows the percentage of living Drosophila controls (dMPC1⁺) or dMPC1 mutants (dMPC⁻) after transfer to standard laboratory medium (std. food) or to media containing only sugar as well as the relative concentrations of ATP, trehalose, glucose, pyruvate, and TCA cycle intermediates between the two groups.

FIG. 3 shows the effect of mpc1Δ, compared to wt, on mitochondrial pyruvate uptake.

FIG. 4 shows the impact of siRNAs targeting either MPC1 or MPC2 on mouse embryonic fibroblasts (MEFs) under basal and FCCP-stimulated conditions.

FIG. 5 shows the alignment of Mpc1 and Mpc2 protein family sequences.

FIG. 6 shows the localization of Mpc1, Mpc2, and Mpc3 to the mitochondrial inner membrane.

FIG. 7 shows the effect of MPC mutations on the ability of yeast to grow on synthetic complete media, synthetic media containing glycerol, or YPAD media.

FIG. 8 shows the localization of dMPC1, the Drosophila ortholog of MPC, via immunofluorescence.

FIG. 9 shows a Northern Blot analysis of RNA isolated from wt Drosophila, homozygotes for a dMPC1 precise excision (dMPC1+) and transheterozygotes for the dMPC1¹/dMPC1² deletion alleles.

FIG. 10 shows the impact of the dMPC1 mutation on the abundance of glycogen, TAG, fructose, and amino acids metabolized through either glycolysis or the TCA cycle in Drosophila.

FIG. 11 shows the relative abundance of metabolites in MPC1, MPC2, and/or MPC3 mutant strains as measured by GC-MS metabolomics.

FIG. 12 shows the growth of pda1 and mpc1 mutants plated on medium lacking leucine.

FIG. 13 shows the effect of siRNAs targeting either MPC1 or MPC2 on pyruvate oxidation and oxidative phosphorylation in mouse embryonic fibroblasts (MEFs).

FIG. 14 shows amplification of cDNA containing the MPC1 open reading frame (ORF) in human skin fibroblasts from a control patient and from patients with impaired pyruvate metabolism.

FIG. 15 shows the effect of mpc1Δ, compared to wt, on mitochondrial pyruvate uptake.

FIG. 16 shows serial dilutions of indicated strains spotted on synthetic media lacking leucine and grown at 30° C. for 48 hours. (wt=wild-type; EV=empty vector).

FIG. 17 shows the impact of siRNAs targeting either MPC1 or MPC2 on mouse embryonic fibroblasts (MEFs) under basal and FCCP-stimulated conditions.

FIG. 18 shows pedigrees of families 1, 2, and 3. Circles indicate females, squares males, and diamonds indicate unknown sex. Black indicates deceased and white living. Arrows mark individuals from whom fibroblasts were obtained. FIG. 18 also shows the protein region of MPC1 containing the predicted amino acid substitutions from all three families aligned by ClustalW.

FIG. 19 shows the rescue by expression of WT MPC1 in mouse embryonic fibroblasts (MEFs) under basal and FCCP-stimulated conditions.

FIG. 20 shows a diagram of a non-competitive inhibitor of MPC relative to mitochondrial metabolic pathways.

FIG. 21 shows a cell assay for purposes of discovering MPC activators.

FIG. 22 shows immunoprecipitations from mitochondrial extracts from mpc1Δmpc2Δ cells expressing Mpc1 and Mpc2 tagged as indicated. FIG. 22 also shows mitochondria extracted from strains harboring either EV or plasmids expressing Mpc1-V5 and Mpc2-HA. FIG. 22 also shows the location of the MPC complex on the cell membrane.

FIG. 23 shows serial dilutions of the indicated yeast strains spotted on synthetic media lacking leucine and grown at 30° C. for 24 hours. FIG. 23 also shows serial dilutions of wild type (wt) and the indicated mutant strains were spotted on synthetic complete media, synthetic media containing glycerol, or YPAD media and grown at 30° C. for 24 hours.

FIG. 24 shows the survival rate and normalized concentration of ATP in wt and dMPC1Δ Drosophila.

FIG. 25 shows the levels of glycolytic intermediates in wt and dMPC1Δ Drosophila.

FIG. 26 shows the levels of TCA Cycle intermediates in wt and dMPC1Δ Drosophila.

FIG. 27 shows the relative abundance of Pyruvate, Acetyl CoA, and CoA in wt and MPG mutant Drosophila.

FIG. 28 shows serial dilutions of the indicated strains carrying the indicated centromeric expression plasmids were plated on medium lacking leucine and grown at 30° C. for 72 hours. FIG. 28 also shows a mitochondrial pyruvate dehydrogenase activity in the indicated strains. p value relative to wt and mpc1Δ.

FIG. 29 shows a diagram of a non-competitive inhibitor of MPC relative to mitochondrial metabolic pathways.

FIG. 30 shows Mae1Δmpc1Δ cells transformed with the indicated plasmid and plated on media containing or lacking combinations of leucine or UK-5099. FIG. 30 also shows the uptake of ¹⁴C-pyruvate into mitochondria isolated from the mpc1Δ strain containing the indicated plasmid in the presence or absence of UK-5099. *** p<0.001, ** p<0.01, * p<0.05, NS=not significant (Student's t test). Mean±SEM.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention can be understood more readily by reference to the following detailed description of the invention and the Examples included therein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats, and that these data, represent endpoints, starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Optional” or “optionally” means that the subsequently described event or circumstance may or may not occur and that the description includes instances where said event or circumstance occurs and instances where it does not.

The word “or” as used herein means any one member of a particular list and also includes any combination of members of that list.

As used herein, “normal” refers to an individual or group of individuals who have not shown any symptoms associated with an aberrant pyruvate metabolism associated condition, or to an individual or group of individuals who have not been diagnosed with an aberrant pyruvate metabolism associated condition. “Normal” also refers to a sample or samples isolated from a normal individual or from group of normal individuals. A sample can be a biological sample. For example, a sample or samples include, but are not limited to, the following: DNA, RNA, mRNA, cDNA, and protein isolated from normal individuals. In an aspect, a “normal” sample can also be defined as a “control” sample or as a “baseline” sample or as “unaffected” sample.

As used herein, the term “determining” can refer to measuring or ascertaining an activity or an event or a quantity or an amount or a change in expression and/or in activity level or in prevalence and/or incidence. For example, determining can refer to measuring or ascertaining the quantity or amount (e.g., the volume, the concentration, the weight, etc. of something, such as amount of MPC1 protein or the amount of MPC2 protein) of aberrant pyruvate metabolism Determining can also refer to measuring or ascertaining the quantity or amount of pyruvate oxidation. Determining can also refer to measuring or ascertaining the quantity or amount of pyruvate oxidation. Determining can also refer to measuring or ascertaining the quantity or amount of MPC1 or MPC2 activity or expression. Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence.

As used herein, mitochondrial pyruvate carrier (MPC) complex means the protein complex comprised of at least the proteins MPC1 and MPC2. The MPC can facilitate the passage of pyruvate into the mitochondrial matrix. As used herein, MPC can also refer to an MPG gene or an individual MPC protein such as the proteins MPC1 and MPC2 (also referred to a MPC1 and MPC2).

As used herein, the MPC1/MPC2 complex means the protein complex comprised of the MPC1 and MPC2 proteins. The MPC1/MPC2 complex can facilitate the passage of pyruvate into the mitochondrial matrix.

### A. DIAGNOSTIC METHODS

Described herein is the MPC1/MPC2 complex, a component of the mitochondrial pyruvate carrier in a variety of animals, including mammals such as humans. The MPC1 and MPC2 proteins have no recognizable homology to any known proteins, particularly any known membrane transporters. One MPC1 subunit and 6-8 MPC2 subunits comprise the intact MPC1/MPC2 complex even though MPC1 and MPC2 are homologous to one another and share many regions of absolute identity throughout eukaryotes.

The identification of the MPC1 and MPC2 proteins as important for mitochondrial pyruvate transport provides a new framework for understanding this level of metabolic control as well as new directions for potential therapeutic intervention.

### 1. Diagnostic Methods Based on MPC Expression or Activity

Described herein are methods of detecting an aberrant pyruvate metabolism associated condition in a subject comprising determining the expression level of MPC1 or MPC2 in a sample from the subject and comparing the expression level to the expression level of MPC1 or MPC2 in a normal sample. Expression levels can be determined from a sample obtained from the subject (e.g. in vitro). In any of the methods describe herein, the expression level of MPC1 or MPC2 can be determined by first amplifying a nucleic acid sample obtained from the subject prior to determining the expression level. If amplification methods are used, the same amplification should be applied to the normal sample for comparative purposes. In addition, once an aberrant pyruvate metabolism associated condition is detected, the method can further comprise administering a therapeutic agent to the subject in order to treat the aberrant pyruvate metabolism associated condition in the subject.

As used herein, the term “subject” means an individual. In an aspect, a subject is a mammal such as a primate, and, more preferably, a human. Non-human primates include marmosets, monkeys, chimpanzees, gorillas, orangutans, and gibbons, to name a few. The term “subject” also includes domesticated animals, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). Subjects can also include, but are not limited to fish (for example, zebrafish, goldfish, tilapia, salmon, and trout), amphibians and reptiles. As used herein, a “subject” is the same as a “patient,” and the terms can be used interchangeably.

As used herein, “aberrant” means differing from the norm or the expected type. Aberrant can be the same as “abnormal” or “anomalous,” and the terms can be used interchangeably. More specifically, as used herein an “aberrant pyruvate metabolism associated condition” can be a condition resulting from an inability to efficiently convert cytosolic pyruvate to mitochondrial acetyl-CoA to drive the TCA cycle and ATP production. An aberrant metabolism associated condition can be, but is not limited to, diabetes, cancer, obesity, heart disease, a neurodegenerative disorder, lactic acidosis, hyperpyruvatemia, cardiomyopathy, or diabetic cardiomyopathy.

In an aspect, an aberrant metabolism associated condition can cause a change in the degree to which carbohydrates are imported into mitochondria and converted into acetyl-CoA, which is a critical step in normal carbohydrate metabolism as well as the onset of diabetes, obesity, and cancer.

In an aspect, a decrease in the expression level of the MPC1 or MPC2 genes below the expression level in the normal sample can indicate the presence of an aberrant pyruvate metabolism associated condition. In another aspect, a change in the ratio of the expression level of the MPC1 or MPC2 genes as compared to the ratio of the expression level in the normal sample can indicate the presence of an aberrant pyruvate metabolism associated condition. By way of example, determining overexpression of either of the MPC1 or MPC2 genes and determining normal expression of the other of the MPC1 or MPC2 genes can indicate the presence of an aberrant pyruvate metabolism associated condition.

Also described herein are methods of detecting an aberrant pyruvate metabolism associated condition in a subject comprising determining the activity of MPC1 or MPC2 in a sample from the subject and comparing the activity level to the activity level of MPC1 or MPC2 in a normal sample. In an aspect, a decrease in the activity of MPC1 or MPC2 below the activity level in the normal sample can indicate the presence of an aberrant pyruvate metabolism associated condition. Any methods known in the art for determining MPC activity can be employed in the methods described herein. In an aspect, MPC1 or MPC2 activity can be determined by measuring pyruvate oxidation. In another aspect, MPC1 or MPC2 activity can be determined by the methods described in Brivet et al. (2003) Mol Genet Metab. 78(3):186-192, which is herein incorporated by reference in its entirety. In addition, once an aberrant pyruvate metabolism associated condition is detected, the method can further comprise administering a therapeutic agent to the subject in order to treat the aberrant pyruvate metabolism associated condition in the subject.

Disclosed herein is a method of detecting an aberrant pyruvate metabolism associated condition in a subject, the method comprising: (a) isolating nucleic acid from a subject; (b) amplifying the nucleic acid isolated in step (a); (c) determining the expression level of MPC1 or MPC2 present in the amplified nucleic acid of step (b) and (d) comparing the expression level obtained in step (c) to the expression level of MPC1 or MPC2 in a normal sample. In an aspect, a disclosed method can comprise isolating RNA from the cells; and synthesizing cDNA from the isolated RNA. In an aspect, determining the expression level of MPC1 or MPC2 in a disclosed method can comprise using the cDNA in a polymerase chain reaction (PCR). In an aspect, a disclosed method can comprise administering a therapeutic agent to the subject and treating the aberrant pyruvate metabolism associated condition in the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) determining the expression level of MPC1 or MPC2; and (e) comparing the expression level obtained in step (d) to the expression level of MPC1 or MPC2 in a normal sample. In an aspect, a decrease in the expression level of MPC1 or MPC2 obtained in step (d) when compared to the expression level of MPC1 or MPC2 in the normal sample can indicate that the subject has an aberrant pyruvate metabolism associated condition. In an aspect of a disclosed method, determining the expression level of MPC1 or MPC2 can comprise using the cDNA in a polymerase chain reaction (PCR). In an aspect, a disclosed method can comprise administering a therapeutic agent to the subject and treating the aberrant pyruvate metabolism associated condition in the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) determining the expression level of MPC1; (e) determining the expression level of MPC2; (f) comparing the expression level obtained in step (d) to the expression level of MPC1 in a normal sample; and (g) comparing the expression level obtained in step (e) to the expression level of MPC1 in a normal sample. In an aspect, a change in the ratio of the expression level of MPC1 obtained in step d and the expression level of MPC2 in step e when compared to the ratio of expression level of the MPC1 and MPC2 in the normal sample can indicate that the subject has an aberrant pyruvate metabolism associated condition. In an aspect, determining the expression level of MPC1 or MPC2 in a disclosed method can comprise using the cDNA in a polymerase chain reaction (PCR). In an aspect, a disclosed method can comprise administering a therapeutic agent to the subject and treating the aberrant pyruvate metabolism associated condition in the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

A method of detecting an aberrant pyruvate metabolism associated condition in a subject, the method comprising: (a) obtaining cells from a subject; (b) isolating protein from the cells; (c) determining the activity level of MPC1 or MPC2; and (d) comparing the activity level obtained in step (c) to the activity level of MPC1 or MPC2 in a normal sample. In an aspect of a disclosed method, a decrease in the activity level of MPC1 or MPC2 obtained in step (c) when compared to the activity level of MPC1 or MPC2 in the normal sample can indicate that the subject has an aberrant pyruvate metabolism associated condition. In an aspect, a disclosed method can comprise administering a therapeutic agent to the subject and treating the aberrant pyruvate metabolism associated condition in the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, determining the activity level of MPC1 or MPC2 or both MPC1 and MPC2 can comprise utilizing one or more immunological-based methods, separation-based methods, protein-based methods, or function-based methods. In an aspect, determining the activity level of MPC1 or MPC2 or both MPC1 and MPC2 can comprise utilizing one or more of the following: electrophoresis, capillary electrophoresis, two-dimensional electrophoresis, chromatography, high performance liquid chromatography, thin layer chromatography, hyperdiffusion chromatography, mass spectrometry, fluid or gel precipitin reactions, single or double immunodiffusion, immunoelectrophoresis, radioimmunoassay, enzyme-linked immunosorbent assays, immunofluorescent assays, and western blotting.

Disclosed herein is a method of detecting an aberrant pyruvate metabolism associated condition in a subject, the method comprising: (a) obtaining cells from a subject; (b) isolating protein from the cells; (c) determining the amount of MPC1 or MPC2; and (d) comparing the amount obtained in step c to the amount of MPC1 or MPC2 in a normal sample. In an aspect, a disclosed method can comprise administering a therapeutic agent to the subject and treating the aberrant pyruvate metabolism associated condition in the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, determining the activity level of MPC1 or MPC2 or both MPC1 and MPC2 can comprise utilizing one or more immunological-based methods, separation-based methods, protein-based methods, or function-based methods. In an aspect, determining the activity level of MPC1 or MPC2 or both MPC1 and MPC2 can comprise utilizing one or more of the following: electrophoresis, capillary electrophoresis, two-dimensional electrophoresis, chromatography, high performance liquid chromatography, thin layer chromatography, hyperdiffusion chromatography, mass spectrometry, fluid or gel precipitin reactions, single or double immunodiffusion, immunoelectrophoresis, radioimmunoassay, enzyme-linked immunosorbent assays, immunofluorescent assays, and western blotting.

### 2. Diagnostic Methods Based on Point Mutations and Resultant Peptides

Described herein are point mutations comprised of multiple variations in the MPC1 or MPC2 genes. One or more of these point mutations are associated with an aberrant pyruvate metabolism condition, or a symptom thereof. Detection of these and other point mutations and sets of point mutations can be useful in designing and performing diagnostic assays for an aberrant pyruvate metabolism associated condition, or a symptom thereof. Point mutations and sets of point mutations can be detected by analysis of nucleic acids, by analysis of polypeptides encoded by MPC1 or MPC2 coding sequences (including polypeptides encoded by splice variants), by analysis of MPC1 or MPC2 non-coding sequences, or by other means known in the art. Analysis of such point mutations and can also be useful in designing prophylactic and therapeutic regimes for an aberrant pyruvate metabolism associated condition, or a symptom thereof. In any of the methods describe herein, the point mutations or variations in the MPC1 or MPC2 can be determined by first amplifying a nucleic acid sample obtained from the subject prior to determining whether a point mutation or variation is present. In addition, once an aberrant pyruvate metabolism associated condition is detected, the method can further comprise administering a therapeutic agent to the subject in order to treat the aberrant pyruvate metabolism associated condition in the subject.

More specifically, described herein are methods of detecting an aberrant pyruvate metabolism associated condition in a subject comprising determining in the subject the identity of one or more point mutations in the MPC1 or MPC2 gene wherein the presence of the one or more mutations indicates the presence of an aberrant pyruvate metabolism associated condition in the subject. In an aspect the methods can comprise determining in the subject the identity of one or more mutations in the MPC1 gene wherein the one or more mutations are 236T>A or 289C>T, and wherein the presence of the one or more point mutations indicates the presence of an aberrant pyruvate metabolism associated condition in the subject. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the methods described herein can further comprise determining the expression or activity of MPC1 or MPC2 in a sample from the subject and comparing the expression or activity levels to the expression or activity levels of MPC1 or MPC2 in a normal sample, wherein a lower or decreased expression or activity of MPC1 or MPC2 below the expression or activity levels in the normal sample indicates the presence of an aberrant pyruvate metabolism associated condition. In another aspect, the methods described herein can further comprise determining the ratio of expression or activity of MPC1 or MPC2 in a sample from the subject and comparing the ratio of expression or activity levels to the ratio of expression or activity levels of MPC1 or MPC2 in a normal sample, wherein a change in the ratio of expression or activity of MPC1 or MPC2 from the ratio of the expression or activity levels in the normal sample indicates the presence of an aberrant pyruvate metabolism associated condition.

Disclosed herein is a method of detecting an aberrant pyruvate metabolism associated condition in a subject, the method comprising: (a) obtaining cells from a subject; (b) isolating RNA from the cells; (c) synthesizing cDNA from the isolated RNA; (d) sequencing the cDNA; (e) identifying one or more mutations in the MPC1 sequence, wherein the one or more mutations is T236A or C289T, and wherein the presence of the one or more mutations indicates that the subject has an aberrant pyruvate metabolism associated condition. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, a T236A mutation can correspond to a Leu79His mutation. In an aspect of a disclosed method, a C289T mutation can correspond to a Arg97Trp mutation.

In an aspect, a disclosed method can comprise (f) determining the expression level of MPC1 or MPC2; and (g) comparing the expression level of MPC1 or MPC2 to the expression level of MPC1 or MPC2 in a normal sample, wherein a decrease in the expression level of MPC1 or MPC2 obtained in step (f) when compared to the expression level of MPC1 or MPC2 in the normal sample can confirm that the subject has an aberrant pyruvate metabolism associated condition. In an aspect of a disclosed method, determining the expression level of MPC1 or MPC2 of step (f) can comprise using the cDNA in a polymerase chain reaction (PCR).

In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

In an aspect, a disclosed method can comprise (f) determining the expression level of MPC1; (g) determining the expression level of MPC2; (h) comparing the expression level obtained in step f to the expression level of MPC1 in a normal sample; and (i) comparing the expression level obtained in step (g) to the expression level of MPC1 in a normal sample; wherein a change in the ratio of the expression level of MPC1 obtained in step (f) and the expression level of MPC2 in step (g) when compared to the ratio of expression level of the MPC1 and MPC2 in the normal sample confirms that the subject has an aberrant pyruvate metabolism associated condition. In an aspect, determining the expression level of MPC1 and determining the expression level of MPC2 can comprise using the cDNA in a polymerase chain reaction (PCR). In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of detecting an aberrant pyruvate metabolism associated condition in a subject, the method comprising: identifying one or more mutations in a MPC1 protein, wherein the one or more mutations is Leu79His or Arg97Trp, and wherein the presence of the one or more mutations indicates that the subject has an aberrant pyruvate metabolism associated condition. In an aspect of a disclosed method, identifying one or more mutations in the MPC1 protein can comprise utilizing one or more antibodies that binds to a mutated MPC1 protein.

Disclosed herein is a method of detecting an aberrant pyruvate metabolism associated condition in a subject, the method comprising: (a) obtaining cells from a subject; (b) isolating protein from the cells; (c) sequencing the isolated protein; (d) identifying one or more mutations in the MPC1 protein, wherein the one or more mutations is Leu79His or Arg97Trp, and wherein the presence of the one or more mutations indicates that the subject has an aberrant pyruvate metabolism associated condition. In an aspect, a disclosed method can comprise (e) determining the activity level of MPC1 or MPC2; and (f) comparing the activity level obtained in step e to the activity level of MPC1 or MPC2 in a normal sample, wherein a decrease in the activity level of MPC1 or MPC2 obtained in step (e) when compared to the activity level of MPC1 or MPC2 in the normal sample can confirm that the subject has an aberrant pyruvate metabolism associated condition. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

As used herein, the term “point mutation” refers to a single base substitution in a gene. A point mutation can be a mutation that results in the replacement of a single base nucleotide with another nucleotide of the genetic material, DNA or RNA. The term point mutation can also include insertions or deletions of a single base pair. The point mutations described herein can be transitions—replacement of a purine base with another purine or replacement of a pyrimidine with another pyrimidine—or transversions—replacement of a purine with a pyrimidine or replacement of a pyrimidine with a purine. In an aspect, a point mutation can be a mutation in which one nucleotide is added, deleted, or replaced by another. Point mutations can also include missense, nonsense, frameshift, and silent mutations. In an aspect, the point mutations described herein can be single nucleotide polymorphisms.

A “set” of point mutations means one or more point mutations, e.g., at least 1, at least 2, at least 3, at least 4, at least 5, at least 6, or more than 6 polymorphisms known, for example, in the MPC1 or MPC2 genes.

As used herein, a “nucleic acid”, “polynucleotide” or “oligonucleotide” is a polymeric form of nucleotides of any length, may be DNA or RNA, and may be single- or double-stranded. Nucleic acids may include promoters or other regulatory sequences. Oligonucleotides are usually prepared by synthetic means. Nucleic acids include segments of DNA, or their complements spanning or flanking any one of the polymorphic sites known in the MPC1 or MPC2 genes. The segments are usually between 5 and 100 contiguous bases and often range from a lower limit of 5, 10, 15, 20, or 25 nucleotides to an upper limit of 10, 15, 20, 25, 30, 50, or 100 nucleotides (where the upper limit is greater than the lower limit). Nucleic acids between 5-10, 5-20, 10-20, 12-30, 15-30, 10-50, 20-50, or 20-100 bases are common. The polymorphic site can occur within any position of the segment. A reference to the sequence of one strand of a double-stranded nucleic acid defines the complementary sequence and except where otherwise clear from context, a reference to one strand of a nucleic acid also refers to its complement.

As used herein, “hybridization probes” are nucleic acids capable of binding in a base-specific manner to a complementary strand of nucleic acid. Such probes include nucleic acids and peptide nucleic acids (Nielsen et al., 1991). Hybridization may be performed under stringent conditions which are known in the art. For example, see Berger and Kimmel (1987) Methods In Enzymology, Vol. 152: Guide To Molecular Cloning Techniques, San Diego: Academic Press, Inc.; Sambrook et al. (1989) Molecular Cloning: A Laboratory Manual, 2nd Ed., Vols. 1-3, Cold Spring Harbor Laboratory; Sambook (2001) 3rd Edition; Rychlik, W. and Rhoads, R. E., 1989, Nucl. Acids Res. 17, 8543; Mueller, P. R. et al. (1993) In: Current Protocols in Molecular Biology 15.5, Greene Publishing Associates, Inc. and John Wiley and Sons, New York; and Anderson and Young, Quantitative Filter Hybridization in Nucleic Acid Hybridization (1985)). As used herein, the term “probe” includes primers. Probes and primers are sometimes referred to as “oligonucleotides.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer but typically ranges from 15 to 30 nucleotides. A primer sequence need not be exactly complementary to a template but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

Exemplary hybridization conditions for short probes and primers is about 5 to 12° C., below the calculated T. Formulas for calculating Tm are known and include: Tₘ=4° C.×(number of G's and C's in the primer)+2° C.×(number of A's and T's in the primer) for oligos<14 bases and assumes a reaction is carried out in the presence of 50 mM monovalent cations. For longer oligos, the following formula can be used: Tm=64.9° C.+41° C.×(number of G's and C's in the primer-16.4)/N, where N is the length of the primer. Another commonly used formula takes into account the salt concentration of the reaction (Rychlik, supra, Sambrook, supra, Mueller, supra.): Tm=81.5° C.+16.6° C.×(log 10[Na+]+[K+])+0.41° C.×(% GC)−675/N, where N is the number of nucleotides in the oligo. The aforementioned formulas provide a starting point for certain applications; however, the design of particular probes and primers may take into account additional or different factors. Methods for design of probes and primers for use in the methods of the invention are well known in the art.

Also described herein are methods of detecting an aberrant pyruvate metabolism associated condition in a subject comprising determining in the subject the identity of one or more mutations in the MPC1 protein, wherein the one or more mutations are Leu79His or Arg97Trp, and wherein the presence of the one or more mutations indicates the presence of an aberrant pyruvate metabolism associated condition in the subject. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the MPC1 protein can be a variant protein.

The term “variant” can refer to a nucleotide sequence in which the sequence differs from the sequence most prevalent in a population, for example by one nucleotide, in the case of the point mutations described herein. For example, some variations or substitutions in the nucleotide sequence of the MPC1 or MPC2 genes can alter a codon so that a different amino acid is encoded resulting in a variant polypeptide. The term “variant,” can also refer to a polypeptide in which the sequence differs from the sequence most prevalent in a population. In an aspect, the polypeptide sequence can differ at a position that does not change the amino acid sequence of the encoded polypeptide (i.e., a conserved change). Variant polypeptides can be encoded by a mutated MPC1 or MPC2. Variant MPC1 or MPC2 polypeptides can be associated with the presence of an aberrant pyruvate metabolism associated condition of the risk of developing an aberrant pyruvate metabolism associated condition.

In an aspect, point mutations can be detected in a target nucleic acid isolated from a subject. Typically genomic DNA is analyzed. For assay of genomic DNA, virtually any biological sample containing genomic DNA or RNA, e.g., nucleated cells, is suitable. Other suitable samples include, but are not limited to, saliva, cheek scrapings, biopsies of retina, kidney or liver or other organs or tissues; skin biopsies; amniotic fluid or CNS samples; and the like. In an aspect RNA or cDNA can be assayed. In an aspect, the assay can detect variant proteins encoded by one or more of the MPC1 or MPC2 genes.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

Two amino acid sequences are considered to have “substantial identity” when they are at least about 80% identical, at least about 90% identical, at least about 95% identical, at least about 98% identical, or at least about 99% identical. Percentage sequence identity is typically calculated by determining the optimal alignment between two sequences and comparing the two sequences. Optimal alignment of sequences may be conducted by inspection, or using the local homology algorithm of Smith and Waterman, 1981, Adv. Appl. Math. 2: 482, using the homology alignment algorithm of Needleman and Wunsch, 1970, J. Mol. Biol. 48: 443, using the search for similarity method of Pearson and Lipman, 1988, Proc. Natl. Acad. Sci. U.S.A. 85: 2444, by computerized implementations of these algorithms (e.g., in the Wisconsin Genetics Software Package, Genetics Computer Group, 575 Science Dr., Madison, Wis.) using default parameters for amino acid comparisons (e.g., for gap-scoring, etc.). It is sometimes desirable to describe sequence identity between two sequences in reference to a particular length or region (e.g., two sequences may be described as having at least 95% identity over a length of at least 500 base pairs). Usually the length will be at least about 50, 100, 200, 300, 400, 500, 600, 700, 800, 900, or 1000 amino acids, or the full length of the reference protein. Two amino acid sequences can also be considered to have substantial identity if they differ by 1, 2, or 3 residues, or by from 2-20 residues, 2-10 residues, 3-20 residues, or 3-10 residues.

It should be understood that mutated sites in the MPC1 or MPC2 genes, can be associated with an aberrant pyruvate metabolism associated condition, or a symptom thereof, wherein the symptom of aberrant pyruvate metabolism associated condition can be, but is not limited to diabetes, cancer, obesity, heart disease, a neurodegenerative disorder, lactic acidosis, hyperpyruvatemia, cardiomyopathy, or diabetic cardiomyopathy. In an aspect, the MPC1 protein can be a variant protein.

Exemplary mutated sites in the MPC1 or MPC2 genes are described herein as examples and are not intended to be limiting. These sites, or point mutations, can also be used in carrying out methods of the invention. Moreover, it will be appreciated that these MPC1 or MPC2 point mutations are useful for linkage and association studies, genotyping clinical populations, correlation of genotype information to phenotype information, loss of heterozygosity analysis, identification of the source of a cell sample and can also be useful to target potential therapeutics to cells.

It will be appreciated that additional mutated sites in the MPC1 or MPC2 genes, which are not explicitly described herein, may further refine this analysis. A point mutation analysis using non-synonymous point mutations in the MPC1 or MPC2 genes can be useful to identify variant MPC1 or MPC2 polypeptides. Point mutations associated with risk may change the level or site of MPC1 or MPC2 expression. It will also be appreciated that a point mutation in the MPC1 or MPC2 genes may be linked to a variation in a neighboring gene. The variation in the neighboring gene may result in a change in expression or form of an encoded protein and have detrimental effects in the carrier.

The methods and materials provided herein can be used to determine whether an MPC1 or MPC2 nucleic acid of a subject (e.g., human) contains a point mutation. For example, methods and materials provided herein can be used to determine whether a subject has a variant point mutation. Any method can be used to detect a point mutation in an MPC1 or MPC2 nucleic acid. For example, point mutations can be detected by sequencing exons, introns, or untranslated sequences, denaturing high performance liquid chromatography (DHPLC), allele-specific hybridization, allele-specific restriction digests, mutation specific polymerase chain reactions, single-stranded conformational polymorphism detection, and combinations of such methods.

Described herein are methods for predicting a subject's risk for having or developing an aberrant pyruvate metabolism associated condition in a human subject, comprising: determining in the subject the identity of one or more point mutations in the MPC1 or MPC2 genes, wherein the one or more point mutations are indicative of the subject's risk for having or developing an aberrant pyruvate metabolism associated condition. In an aspect, the method can predict a subject's risk for having or developing diabetes, cancer, obesity, heart disease, a neurodegenerative disorder, lactic acidosis, hyperpyruvatemia, cardiomyopathy, or diabetic cardiomyopathy.

As used herein, “linkage” describes the tendency of genes, alleles, loci or genetic markers to be inherited together as a result of their location on the same chromosome. Linkage can be measured by percent recombination between the two genes, alleles, loci or genetic markers. Typically, loci occurring within a 50 centimorgan (cM) distance of each other are linked. Linked markers may occur within the same gene or gene cluster. As used herein, “linkage disequilibrium” is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium can be influenced by a number of factors including genetic linkage, the rate of recombination, the rate of mutation, random drift, non-random mating, and population structure. “Linkage disequilibrium” or “allelic association” thus means the non-random association of a particular allele or genetic marker with another specific allele or genetic marker more frequently than expected by chance for any particular allele frequency in the population. A marker in linkage disequilibrium with an informative marker, such as one of the MPC1 or MPC2 point mutations described herein can be useful in detecting susceptibility to an aberrant pyruvate metabolism associated condition, or a symptom thereof. A point mutation that is in linkage disequilibrium with a risk, protective, or otherwise informative point mutation or genetic marker described herein can be referred to as a “proxy” or “surrogate” point mutation. A proxy point mutation may be in at least 50%, 60%, or 70% in linkage disequilibrium with risk, protective, or otherwise informative point mutation or genetic marker described herein, and in an aspect is at least about 80%, 90%, and in another aspect 91%, 92%, 93%, 94%, 95%, 96%, 97%, 98%, 99%, or about 100% in LD with a risk, protective, or otherwise informative point mutation or genetic marker described herein.

Publicly available databases such as the HapMap database (http://ftp.hapmap.org/ld_data/latest/) and Haploview (Barrett, J. C. et al., Bioinformatics 21, 263 (2005)) may be used to calculate linkage disequilibrium between two point mutations. The frequency of identified alleles in disease versus control populations can be determined using the methods described herein. Statistical analyses can be employed to determine the significance of a non-random association between the two point mutations (e.g., Hardy-Weinberg Equilibrium, Genotype likelihood ratio (genotype p value), Chi Square analysis, Fishers Exact test). A statistically significant non-random association between the two point mutations indicates that they are in linkage disequilibrium and that one point mutations can serve as a proxy for the second point mutation.

The discovery that point mutations in the MPC1 or MPC2 genes are associated with an aberrant pyruvate metabolism associated condition, or a symptom thereof, has a number of specific applications including, but not limited to, screening individuals to ascertain risk of developing an aberrant pyruvate metabolism associated condition, or a symptom thereof, and identification of new and optimal therapeutic approaches for individuals afflicted with, or at increased risk of developing, an aberrant pyruvate metabolism associated condition, or a symptom thereof. Without intending to be limited to a specific mechanism, point mutations in the MPC1 or MPC2 genes can contribute to the phenotype of an individual in different ways. Point mutations that occur within the protein coding region of MPC1 or MPC2 may contribute to a phenotype by affecting the protein structure and/or function. Point mutations that occur in the non-coding regions of MPC1 or MPC2 may exert phenotypic effects indirectly via their influence on replication, transcription and/or translation. Certain point mutations in the MPC1 or MPC2 genes may predispose an individual to a distinct mutation that is causally related to an aberrant pyruvate metabolism associated condition, or a symptom thereof. Alternatively, as noted above, a point mutation in the MPC1 or MPC2 genes may be linked to a variation in a neighboring gene. The variation in the neighboring gene may result in a change in expression or form of an encoded protein and have detrimental or protective effects in the carrier.

The identity of bases occupying the point mutation sites in the MPC1 or MPC2 genes can be determined in an individual, e.g., in a patient being analyzed, using any of several methods known in the art. For example, and not to be limiting use of allele-specific probes, use of allele-specific primers, direct sequence analysis, denaturing gradient gel electrophoresis (DGGE) analysis, single-strand conformation polymorphism (SSCP) analysis, and denaturing high performance liquid chromatography (DHPLC) analysis. Other well-known methods to detect point mutations in DNA include use of: Molecular Beacons technology (see, e.g., Piatek et al., 1998; Nat. Biotechnol. 16:359-63; Tyagi, and Kramer, 1996, Nat. Biotechnology 14:303-308; and Tyagi, et al., 1998, Nat. Biotechnol. 16:49-53), Invader technology (see, e.g., Neri et al., 2000, Advances in Nucleic Acid and Protein Analysis 3826:117-125 and U.S. Pat. No. 6,706,471), nucleic acid sequence based amplification (Nasba) (Compton 1991), Scorpion technology (Thelwell et al., 2000, Nuc. Acids Res, 28:3752-3761 and Solinas et al., 2001, “Duplex Scorpion primers in SNP analysis and FRET applications” Nuc. Acids Res, 29:20), restriction fragment length polymorphism (RFLP) analysis, and the like. Additional methods will be apparent to one of skill in the art.

The design and use of allele-specific probes for analyzing polymorphisms are described by e.g., Saiki et al., 1986; Dattagupta, EP 235,726, Saiki, WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes can be designed to hybridize to a segment of target DNA such that the polymorphic site aligns with a central position of the probe.

Allele-specific probes can be used in pairs, one member of a pair designed to hybridize to the reference allele of a target sequence and the other member designed to hybridize to the variant allele. Several pairs of probes can be immobilized on the same support for simultaneous analysis of multiple polymorphisms within the same target gene sequence.

The design and use of allele-specific primers for analyzing polymorphisms are described by, e.g., WO 93/22456 and Gibbs, 1989 Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works best when the polymorphic site is at the extreme 3′-end of the primer, because this position is most destabilizing to elongation from the primer.

In some embodiments, genomic DNA can be used to detect MPC1 or MPC2 point mutations. Genomic DNA is typically extracted from a sample, such as a peripheral blood sample or a tissue sample. Standard methods can be used to extract genomic DNA from a sample, such as phenol extraction. In some cases, genomic DNA can be extracted using a commercially available kit (e.g., from Qiagen, Chatsworth, Calif.; Promega, Madison, Wis.; or Gentra Systems, Minneapolis, Minn.).

Other methods for detecting point mutations can involve amplifying a nucleic acid from a sample obtained from a subject (e.g., amplifying the segments of the MPC1 or MPC2 gene of an individual using MPC1 or MPC2-specific primers) and analyzing the amplified gene. This can be accomplished by standard polymerase chain reaction (PCR & RT-PCR) protocols or other methods known in the art. The amplifying can result in the generation of MPC1 or MPC2 allele-specific oligonucleotides, which span the sites of the point mutations in the MPC1 or MPC2 genes. The MPC1 or MPC2 specific primer sequences and MPC1 or MPC2 allele-specific oligonucleotides can be derived from the coding (exons) or non-coding (promoter, 5′ untranslated, introns or 3′ untranslated) regions of the MPC1 or MPC2 genes. In an aspect Genomic DNA from all subjects can be isolated from peripheral blood leukocytes with QIAamp DNA Blood Maxi kits (Qiagen, Valencia, Calif.). DNA samples can be screened for point mutations in MPC1 or MPC2. Genotyping can be performed by TaqMan assays (Applied Biosystems, Foster City, Calif.) and plates can be read in the 7900HT Fast Real-Time PCR System (Applied Biosystems).

Amplification products generated using PCR can be analyzed by the use of denaturing gradient gel electrophoresis (DGGE). Different alleles can be identified based on sequence-dependent melting properties and electrophoretic migration in solution. See Erlich, ed., PCR Technology, Principles and Applications for DNA Amplification, Chapter 7 (W.H. Freeman and Co, New York, 1992).

Alleles of target sequences can be differentiated using single-strand conformation polymorphism (SSCP) analysis. Different alleles can be identified based on sequence- and structure-dependent electrophoretic migration of single stranded PCR products (Orita et al., 1989). Amplified PCR products can be generated according to standard protocols and heated or otherwise denatured to form single stranded products, which may refold or form secondary structures that are partially dependent on base sequence.

Alleles of target sequences can be differentiated using denaturing high performance liquid chromatography (DHPLC) analysis. Different alleles can be identified based on base differences by alteration in chromatographic migration of single stranded PCR products (Frueh and Noyer-Weidner, 2003). Amplified PCR products can be generated according to standard protocols and heated or otherwise denatured to form single stranded products, which may refold or form secondary structures that are partially dependent on the base sequence.

Direct sequence analysis of polymorphisms can be accomplished using DNA sequencing procedures that are well-known in the art. See Sambrook et al., Molecular Cloning, A Laboratory Manual (2nd Ed., CSHP, New York 1989) and Zyskind et al., Recombinant DNA Laboratory Manual (Acad. Press, 1988).

A wide variety of other methods are known in the art for detecting point mutations in a biological sample. See, e.g., Ullman et al. “Methods for single nucleotide polymorphism detection” U.S. Pat. No. 6,632,606; Shi 2002, “Technologies for individual genotyping: detection of genetic polymorphisms in drug targets and disease genes” Am J Pharmacogenomics 2:197-205; Kwok et al., 2003, “Detection of single nucleotide polymorphisms” Curr Issues Biol. 5:43-60).

Described herein are nucleic acids, disease polymorphic sites, adjacent to or spanning the MPC1 or MPC2 point mutation sites. The nucleic acids can be used as probes or primers (including Invader, Molecular Beacon and other fluorescence resonance energy transfer (FRET) type probes) for detecting MPC1 or MPC2 point mutations.

Also described herein are vectors comprising a nucleotide sequence that encodes a full length MPC1 or MPC2 polypeptide. The vector can also comprise a nucleotide sequence that encodes a sub-domain of the MPC1 or MPC2 polypeptide. Therefore, the MPC1 or MPC2 polypeptides can comprise the amino acid sequence found at MPC1: GenBank: CAI19654.1 (under the name Brain Protein 44 Like (BRP44L)(SEQ ID NO: 17); MPC2: GenBank: CAI20075.1 (under the name Brain Protein 44 (BRP44)(SEQ ID NO: 18), or a variant thereof (e.g., a risk variant or a protective variant) and may be a full-length form or a truncated form. The nucleic acid may be DNA or RNA and may be single-stranded or double-stranded.

Some nucleic acids can encode full-length, variant forms of MPC1 or MPC2 polypeptides. The variant MPC1 or MPC2 polypeptides can differ from the sequences identified under GenBank: CAI19654.1 (SEQ ID NO: 17) or GenBank: CAI20075.1 (SEQ ID NO: 18) at an amino acid encoded by a codon including one of any non-synonymous polymorphic position known in the MPC1 or MPC2 genes. In an aspect, the variant MPC1 or MPC2 polypeptides differ from the sequences identified under GenBank: CAI19654.1 (SEQ ID NO: 17) or GenBank: CAI20075.1 (SEQ ID NO: 18) at an amino acid encoded by a codon including one of the non-synonymous polymorphic positions described herein. It is understood that variant MPC1 or MPC2 genes can be generated that encode variant MPC1 or MPC2 polypeptides that have alternate amino acids at multiple mutated sites in the MPC1 or MPC2 genes.

Expression vectors for production of recombinant proteins and peptides are well known in the art (see Ausubel et al., 2004, Current Protocols In Molecular Biology, Greene Publishing and Wiley-Interscience, New York). Such expression vectors can include the nucleic acid sequence encoding the MPC1 or MPC2 polypeptides linked to regulatory elements, such as a promoter, which drives transcription of the DNA and is adapted for expression in prokaryotic (e.g., E. coli) and eukaryotic (e.g., yeast, insect or mammalian cells) hosts. A variant MPC1 or MPC2 polypeptide can be expressed in an expression vector in which a variant MPC1 or MPC2 gene is operably linked to a promoter. The promoter can be a eukaryotic promoter for expression in a mammalian cell. The transcription regulatory sequences can comprise a heterologous promoter and optionally an enhancer, which is recognized by the host cell. Commercially available expression vectors can be used. Expression vectors can include host-recognized replication systems, amplifiable genes, selectable markers, host sequences useful for insertion into the host genome, and the like.

Also described herein are isolated host cells comprising a vector that encodes a full length MPC1 or MPC2 polypeptide. The host cells can also comprise a vector that encodes a sub-domain of the MPC1 or MPC2 polypeptide. Suitable host cells can include bacteria such as E. coli, yeast, filamentous fungi, insect cells, and mammalian cells, which are typically immortalized, including mouse, hamster, human, and monkey cell lines, and derivatives thereof. Host cells may be able to process the MPC1 or MPC2 gene product to produce an appropriately processed, mature polypeptide. Such processing may include glycosylation, ubiquitination, disulfide bond formation, and the like.

Expression constructs containing an MPC1 or MPC2 gene can be introduced into a host cell, depending upon the particular construction and the target host. Appropriate methods and host cells, both prokaryotic and eukaryotic, are well-known in the art.

Also described herein are mutant organisms comprising mpc1Δ cells and yeast strains. Additionally, described herein are mutant organisms comprising mpc2Δ cells and yeast strains. Furthermore, described herein are organisms comprising mpc3Δ cells and yeast strains. Also described herein are mutant organisms comprising a Drosophila dMPC1 mutant. Further described herein are mutant organisms comprising a Themae1 ΔMPC1 Δ double mutant.

Also described herein are purified or isolated proteins comprising the amino acid sequence of the full length MPC1 or MPC2 polypeptide. In an aspect, the purified or isolated protein can comprise the amino acid sequence of an MPC1 or MPC2 polypeptide sub-domain. A protein assay can be carried out to identify the proteins and to characterize mutations in a subject's MPC1 or MPC2 genes. Methods that can be adapted for detection of the MPC1 or MPC2 proteins are well known. These methods include analytical biochemical methods such as electrophoresis (including capillary electrophoresis and two-dimensional electrophoresis), chromatographic methods such as high performance liquid chromatography (HPLC), thin layer chromatography (TLC), hyperdiffusion chromatography, mass spectrometry, and various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting and others. For example, and not to be limiting, a number of well-established immunological binding assay formats suitable for the practice of the invention are known (see, e.g., Harlow, E.; Lane, D. Antibodies: a laboratory manual. Cold Spring Harbor, N.Y.: Cold Spring Harbor Laboratory; 1988; and Ausubel et al., (2004) Current Protocols in Molecular Biology, John Wiley & Sons, New York N.Y. The assay can be competitive or non-competitive. Typically, immunological binding assays (or immunoassays) utilize a “capture agent” to specifically bind to and, often, immobilize the analyte. In an aspect, the capture agent can be a moiety that specifically binds to a variant MPC1 or MPC2 polypeptide or subsequence. The bound protein may be detected using, for example, a detectably labeled anti-MPC1 or MPC2 antibody. In an aspect, at least one of the antibodies is specific for a variant form of a MPC1 or MPC2 polypeptide. In an aspect, the variant polypeptide can be detected using an immunoblot (Western blot) format.

Also described herein are purified polyclonal antibodies or fragments thereof that bind an MPC1 or MPC2 polypeptide. Also described herein are isolated antibodies or fragments thereof that bind an MPC1 or MPC2 polypeptide.

The antibodies described herein can recognize and hybridize to a reference MPC1 or MPC2 polypeptide or a variant MPC1 or MPC2 polypeptide, in which one or more point mutations are present in the MPC1 or MPC2 coding region. In an aspect, the antibodies can specifically hybridize to variant MPC1 or MPC2 polypeptides or fragments thereof, but not MPC1 or MPC2 polypeptides without a variation at the polymorphic site. The antibodies can be polyclonal, and can be made according to standard protocols. Antibodies can be made by injecting a suitable animal with a wild type or variant MPC1 or MPC2 polypeptide, or fragment thereof, or synthetic peptide fragments thereof.

Also described herein are methods of detecting MPC1 or MPC2 in a subject comprising detecting MPC1 or MPC2 levels using an antibody that specifically hybridizes to MPC1 or MPC2. Methods to identify antibodies that specifically hybridizes to a polypeptide are well-known in the art. For methods, including antibody screening and subtraction methods; see Harlow & Lane, Antibodies, A Laboratory Manual, Cold Spring Harbor Press, New York (1988); Current Protocols in Immunology (J. E. Coligan et al., eds., 1999, including supplements through 2005); Goding, Monoclonal Antibodies, Principles and Practice (2d ed.) Academic Press, New York (1986); Burioni et al., 1998, “A new subtraction technique for molecular cloning of rare antiviral antibody specificities from phage display libraries” Res Virol. 149(5):327-30; Ames et al., 1994, Isolation of neutralizing anti-05a monoclonal antibodies from a filamentous phage monovalent Fab display library. J Immunol. 152(9):4572-81; Shinohara et al., 2002, Isolation of monoclonal antibodies recognizing rare and dominant epitopes in plant vascular cell walls by phage display subtraction. J Immunol Methods 264(1-2):187-94 Immunization or screening can be directed against a full-length protein or, alternatively (and often more conveniently), against a peptide or polypeptide fragment comprising an epitope known to differ between the variant and wild-type forms. Polyclonal antibodies specific for MPC1 or MPC2 polypeptides can be useful in diagnostic assays for detection of the variant forms of MPC1 or MPC2, or as an active ingredient in a pharmaceutical composition.

Also described herein are methods of screening for polymorphic sites in other genes that are in linkage disequilibrium with a point mutation or genetic marker described herein, including but not limited to the point mutations in the MPC1 or MPC2 genes. These methods can involve identifying a polymorphic site in a gene that is in linkage disequilibrium with a point mutation in the MPC1 or MPC2 gene, wherein the point mutation in the MPC1 or MPC2 gene is associated with an aberrant pyruvate metabolism associated condition, or a symptom thereof, (e.g., increased or decreased risk).

Point mutations in the MPC1 or MPC2 genes, such as those described herein, can be used to establish physical linkage between a genetic locus associated with a trait of interest and polymorphic markers that are not associated with the trait, but are in physical proximity with the genetic locus responsible for the trait and co-segregate with it. Mapping a genetic locus associated with a trait of interest facilitates cloning the gene(s) responsible for the trait following procedures described herein or others that are known in the art.

In an aspect, described herein are biological compounds, in particular, proteins, peptides, or nucleic acids, that are differentially present in samples from subjects with an aberrant pyruvate metabolism associated condition, or a symptom thereof, as compared to age-matched control subjects (individuals without the disease). These proteins or conditions can therefore be associated with an aberrant pyruvate metabolism associated condition, or a symptom thereof, and termed an aberrant pyruvate metabolism associated condition-associated biomarkers or biomarkers. In another aspect, these proteins or conditions can be associated with a symptom of an aberrant pyruvate metabolism associated condition, and termed an aberrant pyruvate metabolism associated condition associated biomarkers. In yet another aspect, these proteins or conditions can be associated with the presence or risk of developing an aberrant pyruvate metabolism associated condition and termed an aberrant pyruvate metabolism associated condition-associated biomarkers. These biomarkers can be present at different levels in individuals with an aberrant pyruvate metabolism associated condition, or a symptom thereof, as compared to individuals without the disease. In an aspect, these biomarkers can be present in individuals with an aberrant pyruvate metabolism associated condition, or a symptom thereof, at reduced levels compared to healthy individuals. Exemplary biomarkers shown to be present in individuals with an aberrant pyruvate metabolism associated condition, or a symptom thereof, at reduced levels compared to age-matched control individuals are MPC1 or MPC2. Therefore, described herein are methods of determining MPC1 or MPC2 expression levels in a subject. In practicing the methods described herein, biomarkers can be obtained in a sample, preferably a fluid sample, of the individual. The biomarkers are preferentially obtained in a sample of the individual's saliva, cheek scrapings, biopsies of retina, kidney or liver or other organs or tissues; skin biopsies; amniotic fluid or CNS samples; and the like.

As used herein, the term “biomarker” can refer to a protein found at different levels in a sample from a subject with an aberrant pyruvate metabolism associated condition, or a symptom thereof, compared to a control subject. For example, a “biomarker” can be a protein found at reduced levels in a sample from a subject with an aberrant pyruvate metabolism-associated condition, or a symptom thereof, compared to a control subject. The term “biomarker” can also refer to nucleic acid sequences, for example DNA or RNA sequences, such as the MPC1 or MPC2 nucleic acid sequences described herein.

The biomarkers described herein can be in any form that provides information regarding presence or absence of a variant or point mutation described herein. For example, a disclosed biomarker can be, but is not limited to, a nucleic acid molecule, for example a DNA or RNA molecule, a polypeptide, or an antibody.

The term “level” refers to the amount of a biomarker in a sample obtained from an individual. The amount of the biomarker can be determined by any method known in the art and will depend in part on the nature of the biomarker (e.g., electrophoresis, including capillary electrophoresis, 1- and 2-dimensional electrophoresis, 2-dimensional difference gel electrophoresis DIGE followed by MALDI-ToF mass spectroscopy, chromatographic methods such as high performance liquid chromatography (HPLC), thin layer chromatography (TLC), hyperdiffusion chromatography, mass spectrometry (MS), various immunological methods such as fluid or gel precipitin reactions, single or double immunodiffusion, immunoelectrophoresis, radioimmunoassay (RIA), enzyme-linked immunosorbant assays (ELISA), immunofluorescent assays, Western blotting and others, and enzyme- or function-based activity assays. It is understood that the amount of the biomarker need not be determined in absolute terms, but can be determined in relative terms. For example, the amount of the biomarker may be expressed by its concentration in a sample, by the concentration of an antibody that binds to the biomarker, or by the functional activity (i.e., binding or enzymatic activity) of the biomarker.

The level(s) of a biomarker(s) can be determined as described above for a single biomarker or for a “set” of biomarkers. A set of biomarkers refers to a group of more than one biomarkers that have been grouped together, for example and not for limitation, by a shared property such as their presence at elevated levels in patients with an aberrant pyruvate metabolism associated condition compared to controls, by their presence at reduced levels in patients with an aberrant pyruvate metabolism associated condition compared to controls, by their ratio or difference in levels between patients with an aberrant pyruvate metabolism associated condition and controls (e.g., difference between 1.25- and 2-fold, difference between 2- and 3-fold, difference between 3- and 5-fold, and difference of at least 5-fold), or by function.

The term “difference” as it relates to the level of a biomarker of the invention refers to a difference that is statistically different. A difference is statistically different, for example and not to be limiting, if the expectation is <0.05, the p value determined using the Student's t-test is <0.05, or if the p value determined using the Student's t-test is <0.1. The difference in level of a biomarker between an individual with an aberrant pyruvate metabolism associated condition, or a symptom thereof, and a control individual or population can be, for example and not to be limiting, at least 10% different (1.10 fold), at least 25% different (1.25-fold), at least 50% different (1.5-fold), at least 100% different (2-fold), at least 200% different (3-fold), at least 400% different (5-fold), at least 10-fold different, at least 20-fold different, at least 50-fold different, at least 100-fold different, at least 150-fold, or at least 200-fold different.

MPC1 or MPC2 biomarkers can be detected in any of a number of methods including immunological assays (e.g., ELISA), separation-based methods (e.g., gel electrophoresis), protein-based methods (e.g., mass spectroscopy), function-based methods (e.g., enzymatic or binding activity), or the like. In an aspect, determining MPC1 or MPC2 expression levels comprises using an antibody that specifically binds to MPC1 or MPC2. Other methods are known to those of skill in the art guided by this specification. The particular method for determining the levels will depend, in part, on the identity and nature of the biomarker protein. In an aspect, normal or baseline values (or ranges) can be established for biomarker expression levels. Normal levels can be determined for any particular population, subpopulation, or group of organisms according to standard methods well known to those of skill in the art. Generally, baseline (normal) levels of biomarkers can be determined by quantifying the amount of biomarker in biological samples (e.g., fluids, cells or tissues) obtained from normal (healthy) subjects. Application of standard statistical methods used in medicine permits determination of baseline levels of expression, as well as significant deviations from such baseline levels. It will be appreciated that the assay methods do not necessarily require measurement of absolute values of biomarker, unless it is so desired, because relative values can be sufficient for many applications of the methods described herein. Where quantification is desirable, described herein are reagents such that virtually any known method for quantifying gene products can be used.

In an aspect, the method for separating and determining the levels of the one or more biomarkers described herein, including, but not limited to, MPC1 or MPC2, can involve obtaining a biological sample from an individual, separating and determining the levels of the biomarkers by 2-dimensional difference gel electrophoresis (DIGE), and identifying the biomarkers by MALDI-ToF mass spectroscopy. In another aspect, the biomarkers separated by DIGE can be identified by comparison to a known separation pattern of biomarkers using DIGE.

In a further aspect, the method for separating, detecting, and determining the levels of the biomarkers described herein, including, but not limited to, MPC1 or MPC2, involves obtaining a biological sample from an individual, separating the proteins by chromatography, if appropriate, capturing the proteins on a biochip (i.e., an adsorbent of a SELDI probe), and detecting and determining the levels of the captured biomarkers by mass spectrometry (i.e., ToF-MS).

A biochip can comprise a solid substrate and can have a generally planar surface to which a capture reagent (also called an adsorbent or affinity reagent) can be attached. Frequently, the surface of a biochip comprises a plurality of addressable locations, each of which can have the capture reagent bound thereto.

A “protein biochip” as used herein refers to a biochip adapted for the capture of proteins. Protein biochips are known to those of skill in the art, including, but not limited to, those produced by Ciphergen Biosystems, Inc. (Fremont, Calif.), Packard BioScience Company (Meriden, Conn.), Zyomyx (Hayward, Calif.), Phylos (Lexington, Mass.) and Biacore (Uppsala, Sweden). Examples of such protein biochips are described in, e.g., U.S. Pat. Nos. 6,225,047, 6,329,209 and 5,242,828, and PCT Publication Nos. WO 99/51773 and WO 00/56934.

In an aspect, the biomarkers of the invention can be detected by mass spectrometry (MS) methods. Examples of mass spectrometers include, but are not limited to, time-of-flight (ToF), magnetic sector, quadrupole filter, ion trap, ion cyclotron resonance, electrostatic sector analyzer, and hybrids of these.

In an aspect, the mass spectrometer can be a laser desorption/ionization mass spectrometer. In laser desorption/ionization mass spectrometry, the analytes (i.e., proteins) are placed on the surface of a MS probe, which engages a probe interface of the mass spectrometer and presents an analyte to ionizing energy for ionization and introduction into the mass spectrometer. A laser desorption mass spectrometer employs laser energy, typically from an ultraviolet laser, but also from an infrared laser, which desorbs the analytes from the surface, and volatilizes and ionizes the analytes, thereby making them available to the ion optics of the mass spectrometer.

A mass spectrometry method for use in the methods described herein can be “Surface Enhanced Laser Desorption and Ionization” or “SELDI,” as described, for example, in U.S. Pat. Nos. 5,719,060 and 6,225,047. SELDI refers to a method of desorption/ionization gas phase ion spectrometry in which the analyte (i.e., at least two of the biomarkers) is captured on the surface of a SELDI MS probe. There are several versions of SELDI, including “affinity capture mass spectrometry,” “Surface-Enhanced Affinity Capture” or “SEAC,” “Surface-Enhanced Neat Desorption” or “SEND,” and “Surface-Enhanced Photolabile Attachment and Release” or “SEPAR”.

SEAC involves the use of probes having a material on the probe surface that captures analytes (i.e., proteins) through non-covalent affinity interactions (i.e., adsorption) between the material and the analyte. The material is variously called an “adsorbent,” a “capture reagent,” an “affinity reagent, or a “binding moiety.” Such probes are called “affinity capture probes” having “adsorbent surfaces.” The capture reagent can be any material capable of binding an analyte. The capture reagent can be attached directly to the substrate of the selective surface, or the substrate can have a reactive surface that carries a reactive moiety capable of binding the capture reagent, e.g., through a reaction forming a covalent or coordinate covalent bond. Epoxide and carbodiimidizole can be reactive moieties used to covalently bind protein capture reagents, such as antibodies or cellular receptors. Nitriloacetic acid and iminodiacetic acid can be reactive moieties that function as chelating agents to bind metal ions that interact non-covalently with histidine containing peptides. Adsorbents can be generally classified as either chromatographic adsorbents or biospecific adsorbents.

A “chromatographic adsorbent” refers to an adsorbent material typically used in chromatography. Chromatographic adsorbents include, for example, anion and cation exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A “biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, or a nucleic acid (e.g., DNA)-protein conjugate). In some aspects, the biospecific adsorbent can be a macromolecular structure, such as a multi-protein complex, a biological membrane or a virus. Examples of biospecific adsorbents include, but are not limited to, antibodies, receptor proteins and nucleic acids. Biospecific adsorbents can have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity typically of at least 10⁻⁸ M.

Protein biochips produced by Ciphergen Biosystems, Inc. comprise surfaces having chromatographic or biospecific adsorbents attached thereto at addressable locations. Ciphergen PROTEINCHIP arrays include NP20 (hydrophilic); H4 and HSO (hydrophobic); SAX2, Q10 and LSAX30 (anion exchange); WCX2, CM10 and LWCX30 (cation exchange); IMAC3, IMAC30 and IMAC40 (metal chelate); and PS10, PS20 (reactive surface with carboimidizole, expoxide) and PG20 (protein G coupled through carboimidizole). Hydrophobic PROTEINCHIP arrays have isopropyl or nonylphenoxy-poly(ethylene glycol)methacrylate functionalities. Anion exchange PROTEINCHIP arrays have quaternary ammonium functionalities. Cation exchange PROTEINCHIP arrays have carboxylate functionalities. Immobilized metal chelate PROTEINCHIP arrays have nitriloacetic acid functionalities that adsorb transition metal ions, such as copper, nickel, zinc, and gallium, by chelation. Preactivated PROTEINCHIP arrays have carboimidizole or epoxide functional groups that can react with groups on proteins for covalent binding.

Protein biochips are further described in U.S. Pat. Nos. 6,579,719 and 6,555,813, PCT Publication Nos. WO 00/66265 and WO 03/040700, U.S. Patent Application Nos. US 20030032043 A1, US 20030218130 A1 and US 20050059086 A1.

In an aspect, a probe with an adsorbent surface can be contacted with the sample for a period of time sufficient to allow proteins present in the sample to bind to the adsorbent. After the incubation period, the substrate can be washed to remove unbound material. Any suitable washing solutions can be used; for example, aqueous solutions can be employed. The extent to which proteins remain bound to the adsorbent can be manipulated by adjusting the stringency of the wash. The elution characteristics of a wash solution can depend, for example, on pH, ionic strength, hydrophobicity, degree of chaotropism, detergent strength, temperature, and the like. Unless the probe has both SEAC and SEND properties (as described herein), an energy absorbing molecule can then applied to the substrate with the bound proteins.

The biomarkers bound to the substrates can be detected in a gas phase ion spectrometer such as a ToF mass spectrometer. The biomarkers can be ionized by an ionization source such as a laser, the generated ions can be collected by an ion optic assembly, and then a mass analyzer can disperse and analyze the passing ions. The detector can then translate information of the detected ions into mass-to-charge ratios. Detection of a biomarker can involve detection of signal intensity. Thus, both the quantity and mass of the biomarker can be determined.

SEND involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In an aspect, the EAM can be incorporated into a linear or cross-linked polymer, e.g., a polymethacrylate. For example, the composition can be a co-polymer of α-cyano-4-methacryloyloxycinnamic acid and acrylate. In another aspect, the composition is a co-polymer of α-cyano-4-methacryloyloxycinnamic acid, acrylate and 3-(tri-ethoxy)silyl propyl methacrylate. In another aspect, the composition can be a co-polymer of α-cyano-4-methacryloyloxycinnamic acid and octadecylmethacrylate (“C18 SEND”). SEND is further described in U.S. Pat. No. 6,124,137 and PCT Publication No. WO 03/64594.

SEAC/SEND is a version of SELDI in which both a capture reagent and an EAM can be attached to the sample presenting surface. SEAC/SEND probes can therefore allow the capture of analytes through affinity capture and ionization/desorption without the need to apply an external matrix. The C18 SEND biochip is a version of SEAC/SEND, comprising a C18 moiety which functions as a capture reagent, and a CHCA moiety which functions as an EAM.

SEPAR involves the use of probes having moieties attached to the surface that can covalently bind an analyte, and then release the analyte through breaking a photolabile bond in the moiety after exposure to light, e.g., to laser light (see U.S. Pat. No. 5,719,060). SEPAR and other forms of SELDI can be readily adapted to detecting a biomarker or biomarker profile, pursuant to the methods described herein.

In another MS method, the biomarkers can first be captured on a resin having chromatographic properties that bind biomarkers. This can include a variety of methods. For example, the biomarkers can be captured on a cation exchange resin, such as CM CERAMIC HYPERD F resin, the resin can be washed, biomarkers can be eluted and the eluted biomarkers can be detected by MALDI. Alternatively, this method can be preceded by fractionating the sample on an anion exchange resin, such as Q CERAMIC HYPERD F resin, before application to the cation exchange resin. In another aspect, the sample on an anion exchange resin can be fractionated and detected by MALDI directly. In yet another aspect, the biomarkers can be captured on an immuno-chromatographic resin comprising antibodies that bind particular biomarkers, resin can be washed to remove unbound material, the biomarkers can be eluted from the resin and the eluted biomarkers can be detected by MALDI or by SELDI.

Analysis of analytes by ToF-MS generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data can then be subject to data processing using Ciphergen's PROTEINCHIP software, or any equivalent data processing software. Data processing can include TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program can analyze the data to indicate the number of biomarkers detected, the strength of the signal, or the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which can be set at zero in the scale.

The computer can transform the resulting data into various formats for display. The standard spectrum can be displayed, but in an aspect only the peak height and mass-to-charge information can be retained from the spectrum view, thereby yielding a cleaner image and enabling biomarkers with nearly identical molecular weights to be more easily seen. In another aspect, two or more spectra can be compared, conveniently highlighting unique biomarkers and biomarkers that are up- or down-regulated between samples. Using any of these formats, it can readily be determined whether a particular biomarker is present in a sample.

Analysis can involve the identification of peaks in the spectrum that represent signal from an analyte. Peak selection can be done visually, but software is available, for example, as part of Ciphergen's PROTEINCHIP software package, which can automate the detection of peaks. In general, this software functions by identifying signals having a signal-to-noise ratio above a selected threshold and labeling the mass of the peak at the centroid of the peak signal. In an aspect, many spectra can be compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker described herein. The software also can subject the data regarding observed biomarker peaks to classification tree or ANN analysis, to determine whether a biomarker peak or combination of biomarker peaks is present that indicates the status of the particular clinical parameter under examination. Analysis of the data can be “keyed” to a variety of parameters that are obtained, either directly or indirectly, from the mass spectrometric analysis of the sample. These parameters include, but are not limited to, the presence or absence of at least two peaks, the shape of a peak or group of peaks, the height of at least two peaks, the log of the height of at least two peaks, and other arithmetic manipulations of peak height data.

An example protocol for the detection of biomarkers described herein is as follows. The biological sample to be tested can be obtained a subject, depleted of albumin and IgG or pre-fractionated on an anion exchange chromatographic resin or other chromatographic resin, as appropriate, and then contacted with an affinity capture SELDI probe comprising a cation exchange adsorbant (e.g., CM10 or WCX2 PROTEINCHIP array from Ciphergen Systems, Inc.), an anion exchange adsorbant (e.g., Q10 PROTEINCHIP array from Ciphergen Systems, Inc.), a hydrophobic exchange adsorbant (e.g., HSO PROTEINCHIP array from Ciphergen Systems, Inc.), or an IMAC adsorbant (e.g., IMAC3 or IMAC30 PROTEINCHIP array from Ciphergen Systems, Inc.). The SELDI probe can be washed with a suitable buffer that retains the biomarkers of the invention, while washing away unbound biomolecules. The biomarkers specifically retained on the SELDI probe can then be detected by laser desorption/ionization mass spectrometry.

The biological sample, e.g., serum, plasma or urine, can be depleted of albumin and IgG or subjected to pre-fractionation before binding to a SELDI probe. In an aspect, pre-fractionation can involve contacting the biological sample with an anion exchange chromatographic resin. The bound biomolecules can then be subjected to stepwise pH elution using buffers at various pH. Various fractions containing biomolecules can be collected and subjected to binding to a SELDI probe.

In a further aspect, if analysis of particular proteins and various forms thereof are desired, antibodies which recognize specific proteins can be attached to the surface of a SELDI probe (e.g., pre-activated PS10 or PS20 PROTEINCHIP array from Ciphergen Systems, Inc.). The antibodies capture the target proteins from a biological sample onto the SELDI probe. The captured proteins can then be detected by, for example, laser desorption/ionization mass spectrometry. The antibodies can also capture the target proteins on immobilized support, and the target proteins can be eluted and captured on a SELDI probe and detected as described herein.

Antibodies to target proteins are either commercially available or can be produced by methods known in the art, e.g., by immunizing animals with the target proteins isolated by standard purification techniques or with synthetic peptides of the target proteins.

In some aspects it will be desirable to establish normal or baseline values (or ranges) for biomarker expression levels. Normal levels can be determined for any particular population, subpopulation, or group of organisms according to standard methods well known to those of skill in the art. Generally, baseline (normal) levels of biomarkers are determined by quantifying the amount of biomarker in biological samples (e.g., fluids, cells or tissues) obtained from normal (healthy) subjects. Application of standard statistical methods used in medicine permits determination of baseline levels of expression, as well as significant deviations from such baseline levels.

In carrying out the diagnostic and prognostic methods described herein, it will sometimes be useful to refer to “diagnostic” and “prognostic” values. As used herein, “diagnostic value” refers to a value that is determined for the biomarker gene product detected in a sample which, when compared to a normal (or “baseline”) range of the biomarker gene product is indicative of the presence of a disease. “Prognostic value” refers to an amount of the biomarker that is consistent with a particular diagnosis and prognosis for the disease. The amount of the biomarker gene product detected in a sample is compared to the prognostic value for the biomarker such that the relative comparison of the values indicates the presence of disease or the likely outcome of the disease progression. In an aspect, for example, to assess prognosis of an aberrant pyruvate metabolism associated condition, data are collected to obtain a statistically significant correlation of biomarker levels with different symptoms of an aberrant pyruvate metabolism associated condition. A predetermined range of biomarker levels is established from subjects having known clinical outcomes. A sufficient number of measurements is made to produce a statistically significant value (or range of values) to which a comparison will be made.

It will be appreciated that the assay methods described herein do not necessarily require measurement of absolute values of a biomarker, unless it is so desired, because relative values are sufficient for many applications of the methods described herein. Where quantification is desirable, the presently described methods provide reagents such that one or more of the methods described herein as well as known method for quantifying gene products can be used.

In an aspect, described herein are methods for diagnosing or determining the risk a subject may develop an aberrant pyruvate metabolism associated condition, or a symptom thereof, by determining levels of at least one aberrant pyruvate metabolism associated condition-associated biomarker in a sample from the individual, and comparing the levels of the biomarker in the sample to reference levels of the biomarker characteristic of a control population of individuals without an aberrant pyruvate metabolism associated condition, where a difference in the levels of the biomarker between the sample from the individual and the control population indicates that the individual has or has an increased risk of having an aberrant pyruvate metabolism associated condition, or a symptom thereof. A biomarker can be, but is not limited to, an MPC1 or MPC2 protein. For example, the biomarker can be an MPC1 or MPC2 protein expressed at elevated levels in individuals with an aberrant pyruvate metabolism associated condition, or a symptom thereof. In another aspect, the biomarker can be an MPC1 or MPC2 nucleic acid, such as DNA or RNA.

### B. METHODS OF TREATING AN ABERRANT PYRUVATE METABOLISM ASSOCIATED CONDITION

Described herein are methods for treating a subject with an aberrant pyruvate metabolism associated condition, the method comprising administering to the subject in need thereof an effective amount of one or more MPC1/MPC2 complex modulators in an amount sufficient to ameliorate the aberrant pyruvate metabolism associated condition. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the one or more MPC1/MPC2 complex modulators can be one or more MPC1/MPC2 complex agonists. In another aspect, the one or more MPC1/MPC2 complex modulators can be one or more allosteric MPC inhibitors. In another aspect, the methods for treating a subject with an aberrant pyruvate metabolism associated condition can comprise administering to the subject in need thereof an effective amount of one or more MPC1 or MPC2 modulators in an amount sufficient to ameliorate the aberrant pyruvate metabolism associated condition.

In an aspect, transcriptional regulation of the MPC can play a role in metabolic regulation. Thus, MPC can be regulated post-translationally. Many cancer cells have a high-Km form of the MPC, and the known MPC inhibitors can act non-competitively with a slow onset-of-action. Therefore, in a further aspect, the MPC inhibitors described herein can bind an allosteric site and favor a conformation that is inactive for pyruvate transport.

Also described herein are methods of ameliorating one or more symptoms associated with an aberrant pyruvate metabolism associated condition comprising administering to a subject in need thereof an effective amount of one or more MPC1/MPC2 complex modulators. In an aspect, the one or more MPC1/MPC2 complex modulators can be one or more MPC1/MPC2 complex agonists. In an aspect, the methods of ameliorating one or more symptoms associated with an aberrant pyruvate metabolism associated condition can comprise administering to a subject in need thereof an effective amount of one or more MPC1 or MPC2 modulators.

Disclosed herein is a method for treating a subject diagnosed with an aberrant pyruvate metabolism associated condition, the method comprising: administering to a subject in need thereof an effective amount of a composition comprising one or more MPC1/MPC2 complex modulators; and ameliorating one or more symptoms associated with the aberrant pyruvate metabolism associated condition. In an aspect of a disclosed method, one or more MPC1/MPC2 complex modulators can comprise MPC1/MPC2 complex agonists. In an aspect, one or more MPC1/MPC2 complex modulators can comprise allosteric MPC inhibitors. In an aspect, treatment can restore MPC1 expression in a subject. In an aspect, treatment can restore MPC1 activity in a subject.

In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, diagnosing a subject with an aberrant pyruvate metabolism associated condition can comprises subjecting the subject to a physical examination conducted by a skilled person, such as—for example, a physician, a specialist, or a clinician. In an aspect, a physical examination can identify one or more symptoms associated with the aberrant pyruvate metabolism associated condition. In an aspect of a disclosed method, diagnosing a subject with an aberrant pyruvate metabolism associated condition can comprise utilizing a sample obtained from the subject in one or more biochemical assays and detecting a T236A mutation and/or a C289T mutation.

Disclosed herein is a method for treating a subject diagnosed with an aberrant pyruvate metabolism associated condition, the method comprising: administering to a subject in need thereof an effective amount of a composition comprising MPC1-D118G; and ameliorating one or more symptoms associated with the aberrant pyruvate metabolism associated condition. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

As used herein, “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein the term “ameliorating” means to make or become better or to improve upon. As used herein the term “ameliorating” can also mean to reduce or remove.

The terms “administering” and “administration” refer to any method of providing a pharmaceutical preparation to a subject. Such methods are well known to those skilled in the art and include, but are not limited to, oral administration, sublingual administration, trans-buccal mucosa administration, transdermal administration, administration by inhalation, nasal administration, topical administration, intravaginal administration, ophthalmic administration, intraaural administration, intracerebral administration, intrathecal administration, rectal administration, intraperitoneal administration, and parenteral administration, including injectable such as intravenous administration, intra-arterial administration, intramuscular administration, intradermal administration, and subcutaneous administration. Ophthalmic administration can include topical administration, subconjunctival administration, sub-Tenon's administration, epibulbar administration, retrobulbar administration, intra-orbital administration, and intraocular administration, which includes intra-vitreal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “therapeutically effective amount” or “effective amount” refers to an amount that is sufficient to achieve the desired result or to have an effect on an undesired condition. For example, an “effective amount” can refer to an amount that is sufficient to achieve the desired result, such as inhibiting the development of an aberrant pyruvate metabolism associated condition.

Also described herein are methods of modulating the expression or activity of MPC1 or MPC2 comprising administering to a subject a therapeutically effective amount of one or more of an antisense molecule, an siRNA, a peptide, or a small molecule. For example, modulating can mean either increasing or decreasing the expression or activity of MPC1 or MPC2. In the methods described herein, enhancing MPC1 or MPC2 transcription, or enhancing translation of the MPC1 or MPC2 gene product can modulate the expression or activity of MPC1 or MPC2. Similarly, the activity of an MPC1 or MPC2 gene product (for example, an mRNA, a polypeptide or a protein) can be enhanced, either directly or indirectly. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of MPC1 or MPC2 has not been modulated.

By “modulate,” “modulating,” or “modulated” is meant to alter, by increasing or decreasing.

As used herein, a “modulator” can mean a composition that can either increase or decrease the expression or activity of a gene or gene product such as an MPC1 or MPC2 peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition. For example, a “candidate modulator” can be an active agent, a therapeutic agent, or a pharmaceutical agent.

The term “active agent,” “therapeutic agent,” or “pharmaceutical agent” is defined as an agent, such as a drug, chemotherapeutic agent, chemical compound, etc. For example, and not to be limiting, an active agent, a therapeutic agent, or a pharmaceutical agent can be a naturally occurring molecule or may be a synthetic compound, including, for example and not to be limiting, a small molecule (e.g., a molecule having a molecular weight<1000), a peptide, a protein, an antibody, or a nucleic acid, such as an siRNA or an antisense molecule. An active, therapeutic, or pharmaceutical agent can be used individually or in combination with any other active, therapeutic, or pharmaceutical agent.

In an aspect, the methods described herein can further comprise diagnosing a subject with an aberrant pyruvate metabolism associated condition prior to the administration of a pharmaceutical agent. As used herein, the term “diagnosed” means having been subjected to a physical examination, including but not limited to genetic examination, by a person of skill, for example, a physician, and found to have a condition that can be diagnosed or treated by the compounds, compositions, or methods described herein. For example, “diagnosed with an aberrant pyruvate metabolism associated condition, or a symptom thereof,” means having been subjected to a physical examination by a person of skill, for example, a physician utilizing the methods described herein, and found to have a condition that can be diagnosed as an aberrant pyruvate metabolism associated condition, or a symptom thereof.

As used herein, the term “diagnosed” can also mean having examined a subject's DNA, RNA, or in some cases, protein, to assess the presence or absence of the various SNPs described herein (and, in an aspect, other SNPs and genetic or behavioral characteristics) so as to determine whether the subject has an aberrant pyruvate metabolism associated condition, or a symptom thereof.

Also described herein are methods of modulating the expression or activity of MPC1 or MPC2 comprising administering to a subject a therapeutically effective amount of one or more of an antisense molecule, an siRNA, a peptide, or a small molecule.

Also described herein are methods of treating or alleviating an aberrant pyruvate metabolism associated condition, or a symptom thereof, comprising modulating the expression or activity of MPC1 or MPC2 by administering to a subject a therapeutically effective amount of one or more of an antisense molecule, an siRNA, a peptide, or a small molecule.

In an aspect, described herein are methods for treating a subject with an aberrant pyruvate metabolism associated condition, the method comprising administering to the subject in need thereof an effective amount of a composition comprising MPC1-D118G in an amount sufficient to ameliorate the aberrant pyruvate metabolism associated condition. In an aspect, the aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy.

Also described herein are methods of ameliorating one or more symptoms associated with an aberrant pyruvate metabolism associated condition comprising administering to a subject in need thereof an effective amount of a composition comprising MPC1-D118G in an amount sufficient to ameliorate the one or more symptoms associated with an aberrant pyruvate metabolism associated condition.

The most universal feature of cancer metabolism is known as the Warburg Effect. Cancer cells metabolize glucose to pyruvate through glycolysis, but instead of oxidizing the pyruvate in mitochondria, they convert it to lactate and excrete it. Essentially, cancer cells do not do the reaction catalyzed by the MPC. This is due to decreased expression or activity of the MPC. Thus, many cancer cells can have lower MPC capacity, which can be a result of decreased expression. Many cancers can also have an altered MPC that is less active. For example, and not to be limiting, MPC1 and MPC2 expression decreases ˜20 and ˜10-fold, respectively, upon differentiation of ES cells (which exhibit prototypical “cancer” metabolism). Additionally, expressing MPC1 in cancer cells that have decreased expression of MPC1 can slow the growth rate of the cancer cells.

Therefore, also described herein are methods of treating cancer by inhibiting or reversing the Warburg effect comprising administering to a subject in need thereof an effective amount of one or more MPC1/MPC2 complex modulators in an amount sufficient to inhibit or reverse the Warburg effect. In an aspect, the one or more MPC1/MPC2 complex modulators can be one or more MPC1/MPC2 complex agonists. In another aspect, the methods of inhibiting or reversing the Warburg effect can comprise administering to a subject in need thereof an effective amount of one or more MPC1 or MPC2 modulators.

Further described herein are methods of treating a subject diagnosed with a mitochondrial pyruvate oxidation defect comprising administering to the subject in need thereof an effective amount of one or more pharmaceutical agents, wherein the one or more pharmaceutical agents restore MPC1 expression or activity in the subject. In an aspect the pharmaceutical agent can be an orphan drug. As used herein, “orphan drug” means a pharmaceutical agent that has been developed specifically to treat a rare medical condition, for example, an “orphan disease.” As used herein, an “orphan disease” can be any disease that affects a small percentage of the population, for example, and not to be limiting, a rare genetic disease. Furthermore, as used herein, “mitochondrial pyruvate oxidation defect” means a loss of the activity in cells wherein pyruvate is imported into mitochondria and oxidized to carbon dioxide through the activity of the MPC, pyruvate dehydrogenase and the tricarboxylic acid cycle.

### 1. Antisense Molecules

Described herein are antisense molecules that interact with the disclosed polynucleotides. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437, each of which is herein incorporated by reference in its entirety for its teaching of modifications and methods related to the same.

Generally, the term “antisense” refers to a nucleic acid molecule capable of hybridizing to a portion of an RNA sequence (such as mRNA) by virtue of some sequence complementarity. The antisense nucleic acids described herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane (Letsinger et al., Proc. Natl. Acad. Sci. USA 1989, 86:6553-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988). Additional modifications include those set forth in U.S. Pat. Nos. 6,608,035, 7,176,296; 7,329,648; 7,262,489, 7,115,579; and 7,105,495.

Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine.

Examples of modified sugar moieties include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense molecule is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the host cell.

In a specific example, antisense molecules that recognize a nucleic acid set forth herein, include a catalytic RNA or a ribozyme (for example see WO 90/11364; WO 95/06764; and Sarver et al., Science 247:1222-5, 1990). Conjugates of antisense with a metal complex, such as terpyridylCu (II), capable of mediating mRNA hydrolysis, are described in Bashkin et al. (Appl. Biochem Biotechnol. 54:43-56, 1995). In one example, the antisense nucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

Antisense molecules can be generated by utilizing the Antisense Design algorithm of Integrated DNA Technologies, Inc. (1710 Commercial Park, Coralville, Iowa 52241 USA; (http://www.idtdna.com/Scitools/Applications/AntiSense/Antisense.aspx/).

2. siRNA

Short interfering RNAs (siRNAs), also known as small interfering RNAs, are double-stranded RNAs that can induce sequence-specific post-transcriptional gene silencing, thereby decreasing gene expression (See, for example, U.S. Pat. Nos. 6,506,559, 7,056,704, 7,078,196, 6,107,094, 5,898,221, 6,573,099, and European Patent No. 1.144,623, all of which are hereby incorporated in their entireties by this reference). siRNAs can be of various lengths as long as they maintain their function. In some examples, siRNA molecules are about 19-23 nucleotides in length, such as at least 21 nucleotides, for example at least 23 nucleotides. In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing expression of a gene set forth in Table The effects of siRNAs have been demonstrated in cells from a variety of organisms, including Drosophila, C. elegans, insects, frogs, plants, fungi, mice and humans (for example, WO 02/44321; Gitlin et al., Nature 418:430-4, 2002; Caplen et al., Proc. Natl. Acad. Sci. 98:9742-9747, 2001; and Elbashir et al., Nature 411:494-8, 2001).

Utilizing sequence analysis tools, one of skill in the art can design siRNAs to specifically target any gene set forth herein for decreased gene expression. SiRNAs that inhibit or silence gene expression can be obtained from numerous commercial entities that synthesize siRNAs, for example, Ambion Inc. (2130 Woodward Austin, Tex. 78744-1832, USA), Qiagen Inc. (27220 Turnberry Lane, Valencia, Calif. USA) and Dharmacon Inc. (650 Crescent Drive, #100 Lafayette, Colo. 80026, USA). The siRNAs synthesized by Ambion Inc., Qiagen Inc. or Dharmacon Inc, can be readily obtained from these and other entities by providing a GenBank Accession No. for the mRNA of any gene set forth in Table 1. In addition, siRNAs can be generated by utilizing Invitrogen's BLOCK-IT™ RNAi Designerhttps://rnaidesigner.invitrogen.com/rnaiexpress.

3. shRNA

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e. loop) and about a 19-29 nucleotide sequence that is the reverse complement of the initial target sequence.

### 4. Morpholinos

Morpholinos are synthetic antisense oligos that can block access of other molecules to small (about 25 base) regions of ribonucleic acid (RNA). Morpholinos are often used to determine gene function using reverse genetics methods by blocking access to mRNA. Morpholinos, usually about 25 bases in length, bind to complementary sequences of RNA by standard nucleic acid base-pairing. Morpholinos do not degrade their target RNA molecules. Instead, Morpholinos act by “steric hindrance”, binding to a target sequence within an RNA and simply interfering with molecules which might otherwise interact with the RNA. Morpholinos have been used in mammals, ranging from mice to humans.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety & acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products. Methods of designing, making and utilizing morpholinos are disclosed in U.S. Pat. No. 6,867,349 which is incorporated herein by reference in its entirety.

### 5. Aptamers

Also disclosed are aptamers that interact with the disclosed polynucleotides. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) andtheophylline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of aptamers, the background protein could be ef-1α. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

### 6. Ribozymes

Also disclosed are ribozymes that interact with the disclosed polynucleotides. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrate's sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

### 7. Triplex Forming Functional Nucleic Acid Molecules

Also disclosed are triplex forming functional nucleic acid molecules that interact with the disclosed polynucleotides. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

### 8. External Guide Sequences

Also disclosed are external guide sequences that form a complex with the disclosed polynucleotides. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

### 9. Peptide Nucleic Acid

Also disclosed are polynucleotides that contain peptide nucleic acids (PNAs) compositions. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNAs have 2-aminoethyl-glycine linkages replacing the normal phosphodiester backbone of DNA (Nielsen et al., Science Dec. 6, 1991; 254(5037):1497-500; Hanvey et al., Science. Nov. 27, 1992; 258(5087):1481-5; Hyrup and Nielsen, Bioorg Med Chem. 1996 January; 4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 April; 3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 April; 3(4):437-45; Petersen et al., J Pept Sci. 1995 May-June; 1(3):175-83; Orum et al., Biotechniques. 1995 September; 19(3):472-80; Footer et al., Biochemistry. Aug. 20, 1996; 35(33): 10673-9; Griffith et al., Nucleic Acids Res. Aug. 11, 1995; 23(15):3003-8; Pardridge et al., Proc Natl Acad Sci USA. Jun. 6, 1995; 92(12):5592-6; Boffa et al., Proc Natl Acad Sci USA. Mar. 14, 1995; 92(6):1901-5; Gambacorti-Passerini et al., Blood. Aug. 15, 1996; 88(4):1411-7; Armitage et al., Proc Natl Acad Sci USA. Nov. 11, 1997; 94(23):12320-5; Seeger et al., Biotechniques. 1997 September; 23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993; 65(24):3545-9) and Jensen et al. (Biochemistry. Apr. 22, 1997; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

### 10. Antibodies

As used herein, the term “antibodies” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In an aspect, the “humanized” antibody is a human version of the antibody produced by a germ line mutant animal Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, described herein are humanized versions of an antibody, comprising at least one, two, three, four, or up to all CDRs of a monoclonal antibody that specifically binds to a protein or fragment thereof encoded by a gene set forth herein. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody can comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also can comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies

### 11. Peptides

An MPC1 or MPC2 peptide can be a recombinant MPC1 or MPC2 peptide, a synthetic MPC1 or MPC2 peptide, an MPC1 or MPC2 peptide, a purified MPC1 or MPC2 peptide, or a commercially available MPC1 or MPC2 peptide. An MPC1 or MPC2 peptide can have a non-naturally occurring sequence or can have a sequence present in any species (e.g., human, rat, or mouse). In an aspect, the MPC1 or MPC2 polypeptide can be a variant MPC1 or MPC2 polypeptide. For example, and not to be limiting, the polypeptide can be MPC1-D118G. In some cases, an MPC1 or MPC2 peptide can contain one or more amino acid analogs or other peptidomimetics. As used herein, the term “peptidomimetics” means a molecule that mimics the biological activity of a polypeptide, but that is not peptidic in chemical nature. While, in certain aspects, a peptidomimetic can be a molecule that contains no peptide bonds (that is, amide bonds between amino acids), the term peptidomimetic can include molecules that are not completely peptidic in character, such as pseudo-peptides, semi-peptides and peptoids. Whether completely or partially non-peptide in character, peptidomimetics as described herein can provide a spatial arrangement of reactive chemical moieties that closely resembles the three-dimensional arrangement of active groups in a polypeptide. As a result of this similar active-site geometry, the peptidomimetic can exhibit biological effects that are similar to the biological activity of a polypeptide. The subunits of an MPC1 or MPC2 peptide may be linked by peptide bonds or other bonds such as, for example, ester or ether bonds. An MPC1 or MPC2 peptide can be a full-length MPC1 or MPC2 peptide, a precursor MPC1 or MPC2 peptide, or a fragment of an MPC1 or MPC2 peptide. In some cases, an MPC1 or MPC2 peptide can contain one or more modifications. For example, an MPC1 or MPC2 peptide can be modified to be pegylated or to contain additional amino acid sequences such as an albumin sequence (e.g., a human albumin sequence). In some cases, an MPC1 or MPC2 peptide can be a fusion polypeptide, such as a fusion polypeptide that contains a fragment of an albumin sequence. In some cases, an MPC1 or MPC2 peptide can be covalently attached to oligomers, such as short, amphiphilic oligomers that enable oral administration or improve the pharmacokinetic or pharmacodynamic profile of a conjugated MPC1 or MPC2 peptide. The oligomers can comprise water soluble polyethylene glycol (PEG) and lipid soluble alkyls (short chain fatty acid polymers). See, for example, International Patent Application Publication No. WO 2004/047871 which describes variant and modified peptides and peptide analogs that can be used in the treatment of a variety of conditions. In some cases, an MPC1 or MPC2 peptide can be fused to the Fc domain of an immunoglobulin molecule (e.g., an IgG1 molecule) such that active transport of the fusion polypeptide occurs across epithelial cell barriers via the Fc receptor.

In an aspect, administering an MPC1 or MPC2 peptide to a subject can be designed to produce MPC1 or MPC2 antibodies in the subject. For example, an MPC1 or MPC2 polypeptide that is foreign to a subject's immune system can be administered to the subject so that the subject produces MPC1 or MPC2 antibodies that can inhibit the activity of an MPC1 or MPC2 polypeptide in the subject. Polypeptides that can be administered to the subject include, but are not limited to: MPC1 or MPC2. In a further aspect, a self MPC1 or MPC2 polypeptide can be designed to contain foreign T-cell epitopes so that administration of the polypeptide produces MPC1 or MPC2 antibodies that can inhibit the activity of an MPC1 or MPC2 polypeptide in the subject. Adjuvants such as alum can be used in combination with MPC1 or MPC2 polypeptides. The MPC1 or MPC2 peptide activity can be inhibited by any amount. For example, and not to be limiting, the peptide activity can be inhibited by about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, and by any percentage between about 5% and 100%.

### 12. Small Molecules

Any small molecule that targets, either directly or indirectly, MPC1 or MPC2 nucleic acids or peptides, can be utilized in the methods described herein. These molecules can be identified in the scientific literature, in the StarLite database available from the European Bioinformatics Institute, in DrugBank (Wishart et al. Nucleic Acids Res. 2006 Jan. 1;34 (Database issue):D668-72), package inserts, brochures, chemical suppliers (for example, Sigma, Tocris, Aurora Fine Chemicals, to name a few), or by any other means, such that one of skill in the art makes the association between a small molecule and inhibition of MPC1 or MPC2, either direct or indirect, by the molecule. Preferred small molecules are those small molecules that have IC₅₀ values of less than about 1 mM, less than about 100 micromolar, less than about 75 micromolar, less than about 50 micromolar, less than about 25 micromolar, less than about 10 micromolar, less than about 5 micromolar or less than about 1 micromolar. The half maximal inhibitory concentration (IC₅₀) is a measure of the effectiveness of a compound in inhibiting biological or biochemical function. This quantitative measure indicates how much of a particular compound or other substance (inhibitor) is needed to inhibit a given biological process (or component of a process, i.e., an enzyme, cell, cell receptor or microorganism) by half In other words, it is the half maximal (50%) inhibitory concentration (IC) of a substance (50% IC, or IC₅₀).

### C. METHODS OF DETERMINING THE EFFICACY OF THERAPEUTICS

Described herein are methods of determining in a subject responsiveness to a treatment for an aberrant pyruvate metabolism associated condition, the method comprising: a. determining the expression level of MPC1 or MPC2 in a sample from the subject; b. administering a pharmaceutical agent to the subject; c. determining the expression level of the MPC1 or MPC2 genes in a sample from the subject; and d. comparing the expression level to the expression level of MPC1 or MPC2 prior to administering the pharmaceutical agent to the expression level of the MPC1 or MPC2 genes in the subject after administering the pharmaceutical agent, wherein an increase in the expression level of the MPC1 or MPC2 genes in the subject after administering the pharmaceutical agent indicates responsiveness to the treatment. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the expression level of MPC1 or MPC2 can be increased above the “normal” level of expression.

Also described herein are methods of determining in a subject lack of responsiveness to a treatment for an aberrant pyruvate metabolism associated condition, the method comprising: a. determining the expression level of MPC1 or MPC2 in a sample from the subject; b. administering a pharmaceutical agent to the subject; c. determining the expression level of the MPC1 or MPC2 genes in a sample from the subject; and d. comparing the expression level to the expression level of MPC1 or MPC2 prior to administering the pharmaceutical agent to the expression level of the MPC1 or MPC2 genes in the subject after administering the pharmaceutical agent, wherein no change in the expression level of the MPC1 or MPC2 genes in the subject, or a decrease in the expression level of the MPC1 or MPC2 genes in the subject, after administering the pharmaceutical agent indicates lack of responsiveness to the treatment.

Also described herein are methods of determining in a subject responsiveness to a treatment for an aberrant pyruvate metabolism associated condition, the method comprising: a. determining the activity of MPC1 or MPC2 in a sample from the subject; b. administering a pharmaceutical agent to the subject; c. determining the activity of MPC1 or MPC2 in a sample from the subject; and d. comparing the activity level to the activity level of MPC1 or MPC2 prior to administering the pharmaceutical agent, wherein an increase in the activity of MPC1 or MPC2 in the subject after administering the pharmaceutical agent indicates responsiveness to the treatment. In an aspect, the activity of MPC1 or MPC2 can be increased above the “normal” level of activity.

Further described herein are methods of determining in a subject lack of responsiveness to a treatment for an aberrant pyruvate metabolism associated condition, the method comprising: a. determining the activity of MPC1 or MPC2 in a sample from the subject; b. administering a pharmaceutical agent to the subject; c. determining the activity of MPC1 or MPC2 in a sample from the subject; and d. comparing the activity level to the activity level of MPC1 or MPC2 prior to administering the pharmaceutical agent, wherein no change in the activity of MPC1 or MPC2 in the subject, or a decrease in the activity of MPC1 or MPC2 in the subject, after administering the pharmaceutical agent indicates lack of responsiveness to the treatment.

In an aspect, the methods described herein can further comprise diagnosing a subject with an aberrant pyruvate metabolism associated condition prior to the administration of a pharmaceutical agent. As used herein, the term “diagnosed” means having been subjected to a physical examination, including but not limited to genetic examination, by a person of skill, for example, a physician, and found to have a condition that can be diagnosed or treated by the compounds, compositions, or methods described herein. For example, “diagnosed with an aberrant pyruvate metabolism associated condition, or a symptom thereof,” means having been subjected to a physical examination by a person of skill, for example, a physician utilizing the methods described herein, and found to have a condition that can be diagnosed as an aberrant pyruvate metabolism associated condition, or a symptom thereof.

As used herein, the term “diagnosed” can also mean having examined a subject's DNA, RNA, or in some cases, protein, to assess the presence or absence of the various SNPs described herein (and, in an aspect, other SNPs and genetic or behavioral characteristics) so as to determine whether the subject has an aberrant pyruvate metabolism associated condition, or a symptom thereof.

As used herein, “no increase” or “a decrease” means that there is no significant or perceptible increase in the symptoms of an aberrant pyruvate metabolism associated condition when a subject is examined by a person of ordinary skill using procedures well known in the art, such as the procedures described herein. Treatment can be in the form of administering one or more pharmaceutical agents to the subject alone or in combination with other forms of treatments including, but not limited to, exercise, reducing or eliminating smoking, reducing or eliminating alcohol intake, reducing stress, weight loss, controlling blood pressure, or improving diet and nutritional intake.

Thus, a person of skill in the art can determine whether a course of treatment of an aberrant pyruvate metabolism associated condition, or a symptom thereof, in a subject is effective by following the subject at various time intervals and examining.

A person of skill in the art can determine whether a course of treatment of an aberrant pyruvate metabolism associated condition, or a symptom thereof, in a subject is not effective by following the subject at various time intervals and examining.

Disclosed herein is a method of determining responsiveness to a treatment in a subject diagnosed with an aberrant pyruvate metabolism associated condition, the method comprising: (a) obtaining cells from a subject; (b) isolating RNA from the cells; (c) synthesizing cDNA from the isolated RNA; (d) determining the expression level of MPC1 or MPC2; (e) administering a composition comprising a pharmaceutical agent to the subject; (f) repeating steps (a)-(d); and (g) comparing the expression level MPC1 or MPC2 obtained in step (f) to the expression level of MPC1 or MPC2 obtained in step (d), wherein if the expression level obtained in step (f) is greater than the expression level obtained in step (d), then the subject is responsive to the treatment; wherein if the expression level obtained in step (f) is about equal to the expression level obtained in step (d), then the subject is not responsive to the treatment; and wherein if the expression level obtained in step (f) is less than the expression level obtained in step (d), then the subject is not responsive to the treatment. In an aspect of a disclosed method, if the subject is responsive to the treatment, then the subject's treatment can be continued. In an aspect, continuing treatment can comprise repeating one or more times the administration of a composition. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

In an aspect of a disclosed method, a pharmaceutical agent can be an orphan drug. In an aspect, a pharmaceutical agent can comprise a MPC1/MPC2 complex modulator. In an aspect, a MPC1/MPC2 complex modulator can comprise a MPC1/MPC2 complex agonist. In an aspect, a MPC1/MPC2 complex modulator can comprise an allosteric MPC inhibitor. In an aspect of a disclosed method, treatment can restore MPC1 expression in a subject.

In an aspect, a disclosed method can comprise using the comparison obtained in step (g) to alter the subject's treatment. For example, in an aspect, if a subject is not responsive to the treatment, then the subject's treatment can be discontinued. In an aspect, if a subject is not responsive to the treatment, then a disclosed method can comprise altering an aspect of the composition of step (e) or altering an aspect of the administration of the composition of step (e) and repeating steps (e)-(g). In an aspect of a disclosed method, determining the expression level of MPC1 or MPC2 can comprise using the cDNA in a polymerase chain reaction (PCR). In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of determining responsiveness to a treatment in a subject diagnosed with an aberrant pyruvate metabolism associated condition, the method comprising: (a) obtaining cells from a subject; (b) isolating RNA from the cells; (c) synthesizing cDNA from the isolated RNA; (d) determining the expression level of MPC1 or MPC2; (e) administering a composition comprising a pharmaceutical agent to the subject; (f) repeating steps (a)-(d); and (g) comparing the expression level MPC1 or MPC2 obtained in step (f) to the expression level of MPC1 or MPC2 obtained in step (d), wherein if the expression level obtained in step (f) is greater than the expression level obtained in step (d), then the subject is responsive to the treatment; wherein if the expression level obtained in step (f) is about equal to the expression level obtained in step (d), then the subject is not responsive to the treatment; and wherein if the expression level obtained in step (f) is less than the expression level obtained in step (d), then the subject is not responsive to the treatment. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

In an aspect of a disclosed method, if the subject is responsive to the treatment, then the subject's treatment can be continued. In an aspect, continuing treatment can comprise repeating one or more times the administration of a composition. In an aspect of a disclosed method, treatment can restore MPC1 expression in a subject. In an aspect, a disclosed method can comprise using the comparison obtained in step (g) to alter the subject's treatment. For example, in an aspect, if a subject is not responsive to the treatment, then the subject's treatment can be discontinued. In an aspect, if a subject is not responsive to the treatment, then a disclosed method can comprise altering an aspect of the composition of step (e) or altering an aspect of the administration of the composition of step (e) and repeating steps (e)-(g). In an aspect of a disclosed method, a pharmaceutical agent can be an orphan drug. In an aspect, a pharmaceutical agent can comprise a MPC1/MPC2 complex modulator. In an aspect, a MPC1/MPC2 complex modulator can comprise a MPC1/MPC2 complex agonist. In an aspect, a MPC1/MPC2 complex modulator can comprise an allosteric MPC inhibitor.

In an aspect of a disclosed method, determining the expression level of MPC1 or MPC2 can comprise using the cDNA in a polymerase chain reaction (PCR). In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of determining responsiveness to a treatment in a subject diagnosed with an aberrant pyruvate metabolism associated condition, the method comprising: (a) obtaining cells from a subject; (b) isolating protein from the cells; (c) determining the activity level of MPC1 or MPC2; (d) administering a composition comprising a pharmaceutical agent to the subject; (c) repeating steps (a)-(c); and (f) comparing the activity level of MPC1 or MPC2 obtained in step (e) to the activity level of MPC1 or MPC2 obtained in step (c), wherein if the activity level obtained in step (e) is greater than the activity level obtained in step (c), then the subject is responsive to the treatment, wherein if the activity level obtained in step (e) is about equal to the activity level obtained in step (c), then the subject is not responsive to the treatment; and wherein if the activity level obtained in step (e) is less than the activity level obtained in step (c), then the subject is not responsive to the treatment. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

In an aspect, if a subject is responsive to the treatment, then the subject's treatment can be continued. In an aspect, continuing treatment can comprise repeating one or more times the administration of a composition. In an aspect, a disclosed method can comprise using the comparison obtained in step (f) to alter the subject's treatment. In an aspect, if a subject is not responsive to the treatment, then the subject's treatment can be discontinued. In an aspect, if a subject is not responsive to the treatment, then the method can comprise: altering an aspect of the composition of step (e) or altering an aspect of the administration of the composition of step (e) and repeating steps (e)-(g). In an aspect of a disclosed method, treatment can restore MPC1 expression in a subject. In an aspect, treatment can restore MPC1 activity in a subject. In an aspect, a pharmaceutical agent can be an orphan drug. In an aspect of a disclosed method, a pharmaceutical agent can comprise a MPC1/MPC2 complex modulator. In an aspect, a MPC1/MPC2 complex modulator can comprise a MPC1/MPC2 complex agonist. In an aspect, a MPC1/MPC2 complex modulator can comprise an allosteric MPC inhibitor.

Disclosed herein is a method of determining responsiveness to a treatment in a subject diagnosed with an aberrant pyruvate metabolism associated condition, the method comprising: (a) obtaining cells from a subject; (b) isolating protein from the cells; (c) determining the amount of MPC1 or MPC2; (d) administering a composition comprising a pharmaceutical agent to the subject; (e) repeating steps (a)-(c); and (f) comparing the amount of MPC1 or MPC2 obtained in step (e) to the amount of MPC1 or MPC2 obtained in step (c), wherein if the amount obtained in step (e) is greater than the amount obtained in step (c), then the subject is responsive to the treatment, wherein if the amount obtained in step (e) is about equal to the amount obtained in step (c), then the subject is not responsive to the treatment; and wherein if the amount obtained in step (e) is less than the amount obtained in step (c), then the subject is not responsive to the treatment. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

In an aspect, if a subject is responsive to the treatment, then the subject's treatment can be continued. In an aspect, continuing treatment can comprise repeating one or more times the administration of a composition. In an aspect, a disclosed method can comprise using the comparison obtained in step (f) to alter the subject's treatment. In an aspect, if a subject is not responsive to the treatment, then the subject's treatment can be discontinued. In an aspect, if a subject is not responsive to the treatment, then the method can comprise: altering an aspect of the composition of step (e) or altering an aspect of the administration of the composition of step (e) and repeating steps (e)-(g). In an aspect of a disclosed method, treatment can restore MPC1 expression in a subject. In an aspect, treatment can restore MPC1 activity in a subject. In an aspect, a pharmaceutical agent can be an orphan drug. In an aspect of a disclosed method, a pharmaceutical agent can comprise a MPC1/MPC2 complex modulator. In an aspect, a MPC1/MPC2 complex modulator can comprise a MPC1/MPC2 complex agonist. In an aspect, a MPC1/MPC2 complex modulator can comprise an allosteric MPC inhibitor.

### D. SCREENING METHODS

Furthermore, described herein are methods of screening for an agent or combination of agents effective in treating an aberrant pyruvate metabolism associated condition, or a symptom thereof, in a subject diagnosed with an aberrant pyruvate metabolism associated condition, or a symptom thereof. The methods of screening described herein can also be used to screen for combinations of therapeutic agents in combination with other treatments including, but not limited to, exercise, reducing or eliminating smoking, reducing or eliminating alcohol intake, reducing stress, weight loss, controlling blood pressure, or improving diet and nutritional intake.

Specifically, described herein are methods for screening for a pharmaceutical agent effective in treating an aberrant pyruvate metabolism associated condition comprising: a. determining the expression level of MPC1 or MPC2 in a sample from the subject; b. administering a pharmaceutical agent to a subject; c. determining the expression level of the MPC1 or MPC2 genes in a sample from the subject; and d. comparing the expression level to the expression level of MPC1 or MPC2 prior to administering the pharmaceutical agent to the expression level of the MPC1 or MPC2 genes in the subject after administering the pharmaceutical agent, wherein an increase in the expression level of the MPC1 or MPC2 genes in the subject after administration of the pharmaceutical agent indicates efficacy of the pharmaceutical agent. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the sample from the subject can be cancer cells.

Also described herein are methods of screening for a pharmaceutical agent effective in ameliorating one or more symptoms associated with an aberrant pyruvate metabolism associated condition comprising: a. determining the expression level of MPC1 or MPC2 in a sample from the subject; b. administering a pharmaceutical agent to a subject; c. determining the expression level of the MPC1 or MPC2 genes in a sample from the subject; and d. comparing the expression level to the expression level of MPC1 or MPC2 prior to administering the pharmaceutical agent to the expression level of the MPC1 or MPC2 genes in the subject after administering the pharmaceutical agent, wherein an increase in the expression level of the MPC1 or MPC2 genes in the subject after administration indicates efficacy of the pharmaceutical agent.

Further described herein are methods of screening for a pharmaceutical agent effective in treating an aberrant pyruvate metabolism associated condition comprising: a. determining the activity of MPC1 or MPC2 in a sample from the subject; b. administering a pharmaceutical agent to the subject; c. determining the activity of MPC1 or MPC2 in a sample from the subject; and d. comparing the activity level to the activity level of MPC1 or MPC2 prior to administering the pharmaceutical agent, wherein an increase in the activity of MPC1 or MPC2 in the subject after administration indicates efficacy of the pharmaceutical agent. The aberrant pyruvate metabolism associated condition can be, but is not limited to, diabetes, cancer, obesity, heart disease, a neurodegenerative disorder, lactic acidosis, hyperpyruvatemia, cardiomyopathy, or diabetic cardiomyopathy. In an aspect, the sample from the subject can be cancer cells.

Also described herein are methods of screening for a pharmaceutical agent effective in ameliorating one or more symptoms associated with an aberrant pyruvate metabolism associated condition comprising: a. determining the activity of MPC1 or MPC2 in a sample from the subject; b. administering a pharmaceutical agent to the subject; c. determining the activity of MPC1 or MPC2 in a sample from the subject; and d. comparing the activity level to the activity level of MPC1 or MPC2 prior to administering the pharmaceutical agent, wherein an increase in the activity of MPC1 or MPC2 in the subject after administration indicates efficacy of the pharmaceutical agent.

Also described herein are methods of screening for a drug-resistant MPC mutant organism comprising administering to an organism one or more MPC inhibitors and determining the viability of the organism following the administration of the one or more MPC inhibitors, wherein a viable organism is a drug-resistant MPC mutant organism. In an aspect, the MPC mutant organisms can be an MPC1 mutant organism. In another aspect, the MPC mutant organism can be an MPC2 mutant organism. In a further aspect, the MPC inhibitor used in the methods can be UK-5099 or any MPC inhibitor known in the art.

As used herein, “mutant organism” means an organism arising or resulting from an instance of mutation, for example a base-pair sequence change within the DNA of the organism, which results in the creation or development of a new characteristic or trait not found in the wild type organism. In an aspect, the mutation can be the MPC1 or MPC2 point mutations described herein. Additionally, as used herein “drug-resistant” means the capacity of an organism to withstand or survive exposure to an agent that is toxic to the organism under normal or most circumstances. In an aspect, an organism can acquire drug resistance through a mutation, such as the various MPC1 or MPC2 mutations described herein.

Also described herein are methods of screening for a mutant MPC peptide effective in treating an aberrant pyruvate metabolism associated condition comprising administering to an organism one or more MPC inhibitors, determining the viability of the organism following the administration of the one or more MPC inhibitors, and, wherein the organism is viable, determining the MPC1 or MPC2 peptide sequence from the viable organism, wherein an MPC peptide identified from the viable organism is a mutant MPC peptide effective in treating an aberrant pyruvate metabolism associated condition. In an aspect, the MPC inhibitor can be an MPC1 inhibitor. In another aspect, the MPC1 inhibitor can be UK-5099 or any MPC1 inhibitor known in the art. In a further aspect, the mutant MPC peptide can be a mutant MPC1 peptide. In yet another aspect, the mutant MPC peptide can be a mutant MPC2 peptide. In an aspect, a mutant MPC peptide identified by the screening methods described herein can be useful as a pharmaceutical agent in treating a subject with an aberrant pyruvate metabolism associated condition.

As used herein, a “mutant peptide” can be a polypeptide in which the sequence differs from the sequence most prevalent in a population. In an aspect, the polypeptide sequence can differ at a position that does not change the amino acid sequence of the encoded polypeptide (i.e., a conserved change). Variant polypeptides can be encoded by a mutated MPC1 or MPC2.

Also described herein are methods of screening for a pharmaceutical agent effective in treating a mitochondrial pyruvate oxidation defect comprising administering one or more pharmaceutical agents to a subject, determining the expression or activity of MPC1 or MPC2 in the subject, and comparing those expression or activity levels to the expression or activity levels of MPC1 or MPC2 prior to administering the pharmaceutical agent, wherein an increase in the expression or activity of MPC1 or MPC2 in the subject after administration indicates efficacy of the pharmaceutical agent. In an aspect, the pharmaceutical agent can be an orphan drug.

Disclosed herein is a method of screening for a pharmaceutical agent effective in treating an aberrant pyruvate metabolism associated condition, the method comprising (a) determining the expression level of MPC1 or MPC2 in a first sample from a subject; (b) administering a composition comprising a pharmaceutical agent to the subject; (c) determining the expression level of MPC1 or MPC2 in a second sample from the subject; and (d) comparing the expression level of MPC1 or MPC2 obtained in step (c) to the expression level of MPC1 or MPC2 obtained in step (a); wherein if the expression level obtained in step (c) is greater than the expression level obtained in step (a), then the composition comprising the pharmaceutical agent is effective in treating an aberrant pyruvate metabolism associated condition; wherein if the expression level obtained in step c is about equal to the expression level obtained in step (a), then the composition comprising the pharmaceutical agent is not effective in treating an aberrant pyruvate metabolism associated condition; and wherein if the expression level obtained in step (c) is less than the expression level obtained in step (a), then the composition comprising the pharmaceutical agent is not effective in treating an aberrant pyruvate metabolism associated condition. In an aspect, a decrease in the expression level of MPC1 or MPC2 obtained in step (d) when compared to the expression level of MPC1 or MPC2 in the normal sample can indicate that the subject has an aberrant pyruvate metabolism associated condition. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

In an aspect, a disclosed method can comprise using the pharmaceutical agent identified in step (d) to treat the subject. In an aspect, a pharmaceutical agent can ameliorate one or more symptoms associated with an aberrant pyruvate metabolism associated condition. In an aspect, treatment can restore MPC1 expression in the subject. In an aspect, a pharmaceutical agent can be an orphan drug. In an aspect, a pharmaceutical agent can be a MPC1/MPC2 complex modulator. In an aspect, a MPC1/MPC2 complex modulator can be a MPC1/MPC2 complex agonist. In an aspect, a MPC1/MPC2 complex modulator can be an allosteric MPC inhibitor.

In an aspect of a disclosed method, determining the expression level of MPC1 or MPC2 in a first sample from a subject can comprise isolating nucleic acid from a subject and amplifying the nucleic acid isolated in step (a). In an aspect of a disclosed method, determining the expression level of MPC1 or MPC2 in a second sample from a subject can comprises isolating nucleic acid from a subject and amplifying the nucleic acid isolated in step (a). In an aspect of a disclosed method, determining the expression level of MPC1 or MPC2 in a first sample from a subject can comprise obtaining cells from a subject; isolating RNA from the cells; synthesizing cDNA from the isolated RNA; and using the cDNA in a polymerase chain reaction (PCR). In an aspect of a disclosed method, determining the expression level of MPC1 or MPC2 in a second sample from the subject can comprise obtaining cells from a subject; isolating RNA from the cells; synthesizing cDNA from the isolated RNA; and using the cDNA in a polymerase chain reaction (PCR).

In an aspect of a disclosed method, PCR can comprise at least one primer. In an aspect of a disclosed method, PCR can comprise several primers. In an aspect, the at least one primer can be a forward primer. In an aspect, the at least one primer can be a reverse primer. In an aspect, when the at least one primer is a forward primer, PCR can further comprise at least one reverse primer. In an aspect, one or more primers can be specific for a target of interest. For example, in a method disclosed herein, one or more primers can be specific for MPC1. In an aspect, one or more primers are specific for MPC2. In an aspect, one or more primers can be specific for a mutant or variant MPC1. In an aspect, one or more primers can be specific for a mutant or variant MPC2. In an aspect, a disclosed forward primer can comprise the sequence set forth in SEQ ID NO:9. In an aspect, a disclosed reverse primer can comprise the sequence set forth in SEQ ID NO:10.

Disclosed herein is a method of screening for a pharmaceutical agent effective in treating an aberrant pyruvate metabolism associated condition, the method comprising (a) determining the activity level of MPC1 or MPC2 in a first sample from a subject; (b) administering a composition comprising a pharmaceutical agent to the subject; (c) determining the activity level of MPC1 or MPC2 in a second sample from the subject; and (d) comparing the activity level of MPC1 or MPC2 obtained in step (c) to the activity level of MPC1 or MPC2 obtained in step (a), wherein if the activity level of obtained in step (c) is greater than the activity level obtained in step (a), then the composition comprising the pharmaceutical agent is effective in treating an aberrant pyruvate metabolism associated condition; wherein if the activity level obtained in step (c) is about equal to the activity level obtained in step (a), then the composition comprising the pharmaceutical agent is not effective in treating an aberrant pyruvate metabolism associated condition; and wherein if the activity level obtained in step (c) is less than the activity level obtained in step (a), then the composition comprising the pharmaceutical agent is not effective in treating an aberrant pyruvate metabolism associated condition. In an aspect, a disclosed method can comprise using the pharmaceutical agent identified in step (d) to treat the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, treatment can restore MPC1 activity in the subject. In an aspect, a pharmaceutical agent can be an orphan drug. In an aspect, a pharmaceutical agent can be a MPC1/MPC2 complex modulator. In an aspect, a MPC1/MPC2 complex modulator can be a MPC1/MPC2 complex agonist. In an aspect, a MPC1/MPC2 complex modulator can be an allosteric MPC inhibitor.

Disclosed herein is a method of screening for a pharmaceutical agent effective in treating an aberrant pyruvate metabolism associated condition, the method comprising: (a) determining the amount of MPC1 or MPC2 in a first sample from a subject; (b) administering a composition comprising a pharmaceutical agent to the subject; (c) determining the amount of MPC1 or MPC2 in a second sample from the subject; and (d) comparing the amount of MPC1 or MPC2 obtained in step (c) to the amount of MPC1 or MPC2 obtained in step (a), wherein if the amount of obtained in step c is greater than the activity level obtained in step a, then the composition comprising the pharmaceutical agent is effective in treating an aberrant pyruvate metabolism associated condition; wherein if the amount obtained in step (c) is about equal to the amount obtained in step (a), then the composition comprising the pharmaceutical agent is not effective in treating an aberrant pyruvate metabolism associated condition; and wherein if the amount obtained in step (c) is less than the activity level obtained in step (a), then the composition comprising the pharmaceutical agent is not effective in treating an aberrant pyruvate metabolism associated condition. In an aspect, a disclosed method can comprise using the pharmaceutical agent identified in step (d) to treat the subject. In an aspect, an aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity. In an aspect of a disclosed method, treatment can restore MPC1 activity in the subject. In an aspect, a pharmaceutical agent can be an orphan drug. In an aspect, a pharmaceutical agent can be a MPC1/MPC2 complex modulator. In an aspect, a MPC1/MPC2 complex modulator can be a MPC1/MPC2 complex agonist. In an aspect, a MPC1/MPC2 complex modulator can be an allosteric MPC inhibitor.

### 1. Methods of Screening for MPC Agonists or Activators

Described herein are methods of screening for an MPC agonist or activator comprising administering to a cell an effective amount of one or more compositions and determining pyruvate oxidation in the cell, wherein an increase in pyruvate oxidation is indicative of an MPC agonist or activator. The cell can be any cell known in the art that can be utilized in a pyruvate oxidation assay. For example, and not to be limiting, the cell can be a cancer cell. In an aspect, the MPC agonist or activator can be an MPC1 agonist or activator. In another aspect, the MPC agonist or activator can be an MPC2 agonist or activator. In yet another aspect, the MPC agonist or activator can be an MPC1/MPC2 complex agonist or activator. In still another aspect, the one or more compositions can be modified allosteric MPC inhibitors.

Pyruvate oxidation can be determined by any methods known in the art. In an aspect, pyruvate oxidation can be determined by a colormetric assay. In an aspect, the colormetric assay can be one of the various commercially available colormetric assays including, but not limited to, the Pyruvate Determination assay kit (Bio Vision, Mountain View, Calif.), or the Pyruvate Assay kit available from Eton Bioscience, Inc. In another aspect, pyruvate oxidation can be determined by the fluorimetric assay described by Zhu, et al. in Analytical Biochemistry 396 (2010) 146-151, which is herein incorporated in its entirety by this reference.

In an aspect, cell-free methods can be employed to screen for an MPC agonist or activator. Such cell-free screening methods can comprise directly determining pyruvate uptake into mitochondria or reconstituted vesicles following the administration of one or more compositions. In an aspect, the cell-free screening methods can comprise determining pyruvate oxidation in purified mitochondria.

Disclosed herein is a method of screening for a drug-resistant MPC mutant organism, the method comprising (a) administering to an organism a composition comprising one or more MPC inhibitors; and (b) determining the viability of the organism following the administration of the composition, wherein a viable organism is a drug-resistant MPC mutant organism. In an aspect, a MPC mutant can be a MPC1 mutant. In an aspect, a MPC mutant can be a MPC2 mutant. In an aspect, one or more MPC inhibitors can comprise UK-5099.

Disclosed herein is a method of screening for a mutant MPC peptide effective in treating an aberrant pyruvate metabolism associated condition, the method comprising (a) administering to an organism a composition comprising one or more MPC inhibitors; (b) determining the viability of the organism following the administration of the composition, wherein if the organism is viable, then (i) isolating the MPC1 or MPC2 peptide from the viable organism, and (ii) sequencing the isolated MPC1 or MPC2 peptide, wherein the sequenced MPC peptide is a mutant MPC peptide effective in treating an aberrant pyruvate metabolism associated condition. In an aspect, one or more MPC inhibitors can comprise a MPC1 inhibitor. In an aspect, a MPC1 inhibitor can comprise UK-5099. In an aspect, a mutant MPC peptide can be a mutant MPC1 peptide. In an aspect, a mutant MPC peptide can be a mutant MPC2 peptide. In an aspect, the aberrant pyruvate metabolism associated condition can comprise cancer, heart disease, a neurodegenerative disorder, lactic acidosis, hyperpyruvatemia, diabetes, diabetic cardiomyopathy, cardiomyopathy, and obesity.

Disclosed herein is an in vitro method of screening for an MPC modulator, the method comprising: administering to a cell an effective amount of a composition comprising a candidate MPC modulator; and determining the level of pyruvate oxidation in the cell following administration of the composition, wherein an increase in the level of pyruvate oxidation indicates that the candidate MPC modulator is an MPC modulator. In an aspect, a MPC modulator can be a MPC1 modulator. In an aspect, a MPC modulator can be a MPC2 modulator. In an aspect, a MPC modulator can be a MPC1/MPC2 complex modulator. In an aspect, a MPC modulator can be a modified allosteric MPC inhibitor. In an aspect of a disclosed method, determining the level of pyruvate oxidation can comprise using a colorimetric assay. In an aspect, a cell is a cancer cell.

### E. ORGANISMS

Disclosed herein is a mutant organism comprising MPC1A cells and yeast strains. Disclosed herein is a mutant organism comprising MPC2A cells and yeast strains. Disclosed herein is a mutant organism comprising mpc3Δ cells and yeast strains. Disclosed herein is a mutant organism comprising a Drosophila dMPC1 mutant. Disclosed herein is a mutant organism comprising a Themae1 ΔMPC1 Δ double mutant.

### F. THERAPEUTIC COMPOSITIONS

Described herein are the components to be used to prepare the compositions of the invention as well as the compositions themselves to be used within the methods described herein. These and other materials are described herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular indicator reagent is disclosed and discussed and a modification of that indicator reagent is also discussed, specifically contemplated is each and every combination and permutation of the indicator reagent and the modifications that are possible unless specifically indicated to the contrary. Thus, if a class of indicator reagents A, B, and C are disclosed as well as a class of indicator reagents D, E, and F and an example of a combination indicator reagent, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed, it is understood that each of these additional steps can be performed with any specific embodiment or combination of embodiments of the methods of the invention.

The compositions described herein have certain functions, such as treating an aberrant pyruvate metabolism associated condition or ameliorating a symptom associated with an aberrant pyruvate metabolism associated condition. Described herein are certain structural requirements for performing the disclosed functions, and it is understood that there are a variety of structures that can perform the same function that are related to the disclosed structures, and that these structures will typically achieve the same result, for example treating an aberrant pyruvate metabolism associated condition, or a symptom thereof.

In an aspect, the pharmaceutical agents to treat an aberrant pyruvate metabolism associated condition, or a symptom thereof, include biological therapies such as gene therapy. For example, DNA containing all or part of the coding sequence for an MPC1 or MPC2 polypeptide can be incorporated into a vector for expression of the encoded polypeptide in suitable host cells. In an aspect, the coding sequence for an MPC1 or MPC2 polypeptide can encode a variant MPC1 or MPC2 polypeptide. For example, and not to be limiting, the polypeptide can be MPC1-D118G. A large number of vectors, including bacterial, yeast, and mammalian vectors, are known in the art for replication and/or expression in various host cells or cell-free systems, and may be used for gene therapy. Expression vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). For example, disclosed herein are expression vectors comprising an isolated polynucleotide comprising a sequence of one or more of genes described herein, operably linked to a control element.

As used herein, plasmid or viral vectors are agents that transport the disclosed nucleic acids, such as an isolated polynucleotide capable of encoding one or more polypeptides disclosed herein into the cell without degradation and include a promoter yielding expression of the gene in the cells into which it is delivered. In some embodiments the isolated polynucleotides disclosed herein are derived from either a virus or a retrovirus.

### 1. Pharmaceutical Carriers/Delivery of Pharmaceutical Products

The compositions described herein (alternatively referred to as compositions) can also be administered in vivo in a pharmaceutically acceptable carrier. By “pharmaceutically acceptable” is meant a material that is not biologically or otherwise undesirable, i.e., the material may be administered to a subject, along with an agent, for example, a peptide, described herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered by oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. As used herein, “topical intranasal administration” means delivery of the compositions into the nose and nasal passages through one or both of the areas and can comprise delivery by a spraying mechanism or droplet mechanism, or through aerosolization. Administration of the compositions by inhalant can be through the nose or mouth via delivery by a spraying or droplet mechanism. Delivery can also be directly to any area of the respiratory system (e.g., lungs) via intubation. The compositions can be administered ophthalmicly.

The exact amount of the compositions required will vary from subject to subject, depending on the species, age, weight and general condition of the subject, the severity of the disease, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. In a further aspect, the disclosed compositions are administered by I.V., by injection and/or an I.V. drip.

a. Pharmaceutically Acceptable Carriers

The compositions described herein can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8, and more preferably from about 7 to about 7.5. Further carriers include sustained release preparations such as semipermeable matrices of solid hydrophobic polymers containing the peptide, which matrices are in the form of shaped articles, e.g., films, liposomes, nanoparticles, or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, Ringer's solution, dextrose in water, balanced salt solutions, and buffered solutions at physiological pH. The pharmaceutical carrier employed can also be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Pharmaceutical compositions may include carriers, thickeners, diluents, buffers, preservatives, surface active agents and the like in addition to the one or more molecules of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono, di-, trialkyl and aryl amines and substituted ethanolamines.

Certain materials, compounds, compositions, and components described herein can be obtained commercially or readily synthesized using techniques generally known to those of skill in the art.

b. Dosages

Effective dosages and schedules for administering the compositions described herein may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the aberrant pyruvate metabolism associated condition, or a symptom thereof, in the patient, route of administration, or whether other drugs are included in the regimen, and can be determined by one of skill in the art. The dosage can be adjusted by the individual physician in the event of any contraindication. Dosage can vary and can be administered in one or more dose administrations daily for one or several days. Guidance can be found in the literature for appropriate dosages for given classes of pharmaceutical products, particularly peptides. Examples of such guidance can be found throughout the literature.

In an aspect, the dosage level can be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

### G. ARRAYS

Also described herein are arrays comprising polynucleotides capable of specifically hybridizing to an MPC1 or MPC2 gene or a variant MPC1 or MPC2 encoding nucleic acid. For example, described are arrays comprising polynucleotides capable of specifically hybridizing to one or more point mutations described herein.

Also described herein are solid supports comprising one or more polypeptides capable of specifically hybridizing to MPC1 or MPC2 or a variant MPC1 or MPC2 peptide.

Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such as capture compounds, can also be immobilized on solid supports. For example, described herein are antigen binding agents capable of specifically binding to an MPC1 or MPC2 peptide or a variant MPC1 or MPC2 peptide.

A preferred form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, nanoparticles, microparticles, or a combination. Solid-state substrates and solid supports can be porous or non-porous. A preferred form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides described herein.

Although preferred, it is not required that a given array be a single unit or structure. The set of compounds may be distributed over any number of solid supports. For example, at one extreme, each compound may be immobilized in a separate reaction tube or container, or on separate beads or microparticles or nanoparticles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second, detection compound, such as an antibody. The detection compound can be specific for the same or a different protein on the calcifying nano-particle.

Methods for immobilizing antibodies (and other proteins) to solid-state substrates are well established Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy] succinimide ester (GMBS). These and other attachment agents, as well as methods for their use in attachment, are described in Protein immobilization: fundamentals and applications, Richard F. Taylor, ed. (M. Dekker, New York, 1991); Johnstone and Thorpe, Immunochemistry In Practice (Blackwell Scientific Publications, Oxford, England, 1987) pages 209-216 and 241-242, and Immobilized Affinity Ligands; Craig T. Hermanson et al., eds. (Academic Press, New York, 1992) which are incorporated by reference in their entirety for methods of attaching antibodies to a solid-state substrate. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A preferred method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each of the other different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support is the sequences or part of the sequences set forth in any of the nucleic acid sequences described herein. Also disclosed are solid supports where at least one address is the sequences or portion of sequences set forth in any of the peptide sequences described herein. Solid supports can also contain at least one address is a variant of the sequences or part of the sequences set forth in any of the nucleic acid sequences described herein. Solid supports can also contain at least one address as a variant of the sequences or portion of sequences set forth in any of the peptide sequences described herein.

Also disclosed are antigen microarrays for multiplex characterization of antibody responses. For example, disclosed are antigen arrays and miniaturized antigen arrays to perform large-scale multiplex characterization of antibody responses directed against the polypeptides, polynucleotides and antibodies described herein, using submicroliter quantities of biological samples as described in Robinson et al., Autoantigen microarrays for multiplex characterization of autoantibody responses, Nat Med., 8(3):295-301 (2002), which is herein incorporated by reference in its entirety for its teaching of constructing and using antigen arrays to perform large-scale multiplex characterization of antibody responses directed against structurally diverse antigens, using submicroliter quantities of biological samples.

Protein variants and derivatives are well understood to those of skill in the art and can involve amino acid sequence modifications. For example, amino acid sequence modifications typically fall into one or more of three classes: substitutional, insertional or deletional variants. Polypeptide variants described herein will typically exhibit at least about 70%, 75%, 80%, 85%, 90%, 91%, 92%, 93%, 94%, 95%, 96%, 97%, 98%, or 99% or more identity (determined as described below), along their length, to the polypeptide sequences set forth herein.

### H. EXAMPLE

The following example is put forth so as to provide those of ordinary skill in the art with a complete disclosure and description of how the compounds, compositions, articles, devices and/or methods claimed herein are made and evaluated, and is intended to be purely exemplary of the invention and is not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example

Pyruvate occupies a node in the regulation of carbon metabolism as it is the end product of glycolysis and a major substrate for the tricarboxylic acid (TCA) cycle in mitochondria. Pyruvate lies at the intersection of these catabolic pathways with anabolic pathways for lipid synthesis, amino acid biosynthesis, and gluconeogenesis. As a result, the failure to correctly partition carbon between these fates influences the altered metabolism evident in diabetes, obesity and cancer [Hanahan et al., 2011; Kahn et al., 2006]. Due to the importance of pyruvate, the mitochondrial pyruvate carrier (MPC) has been studied [Halestrap 1974, 1978]. This included the discovery that α-cyanocinnamate analogs, such as UK-5099, act as specific and potent inhibitors of carrier activity [Halestrap 1975]. In spite of this characterization, however, the gene or genes that encode the mitochondrial pyruvate carrier remain unknown [Todisco et al., 2006; Kildyard et al., 2003].

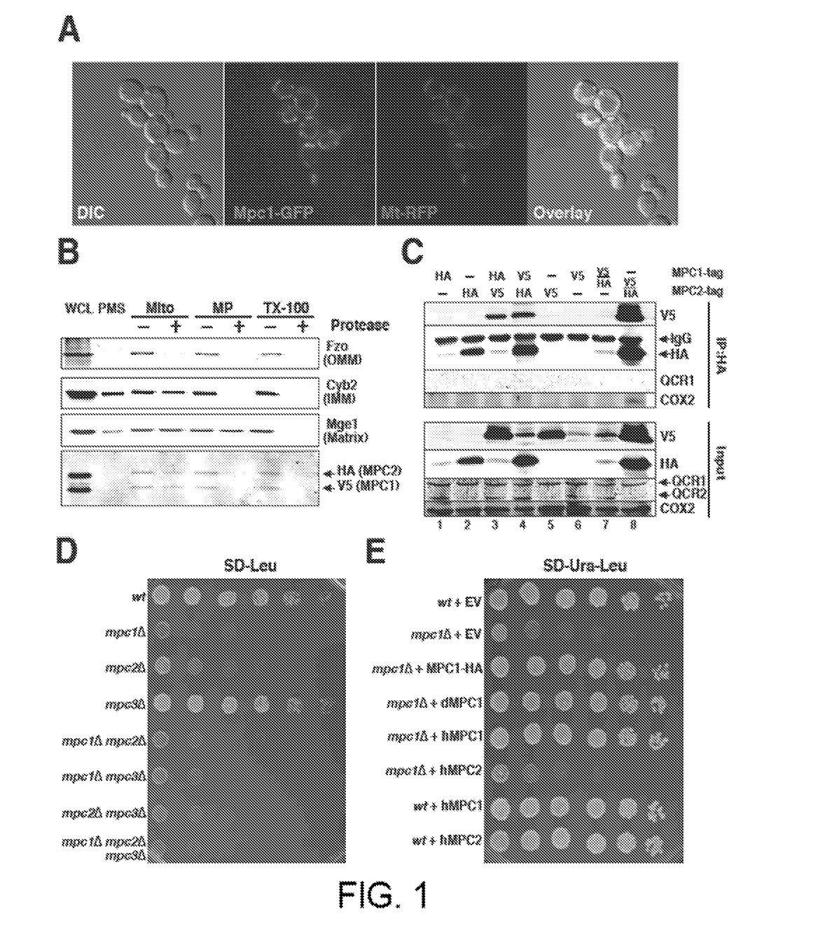

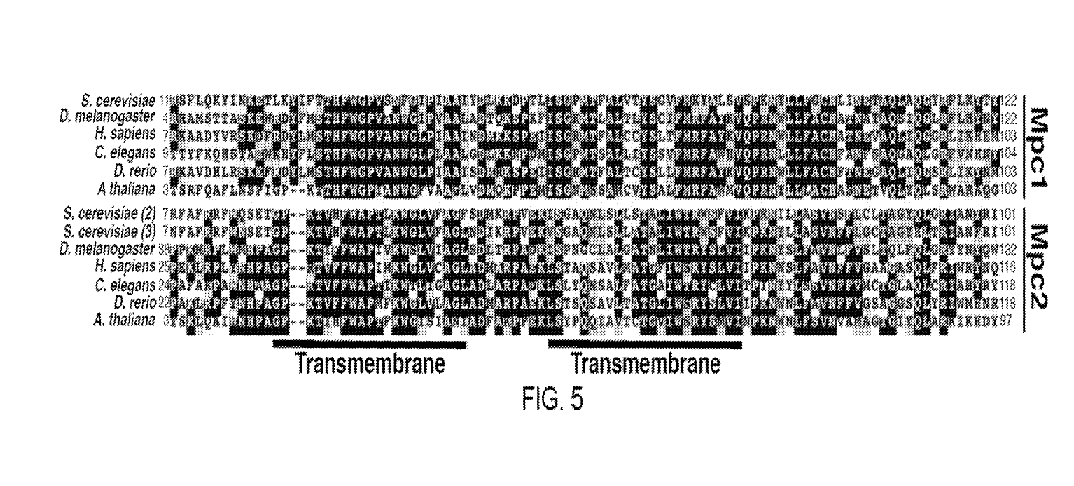

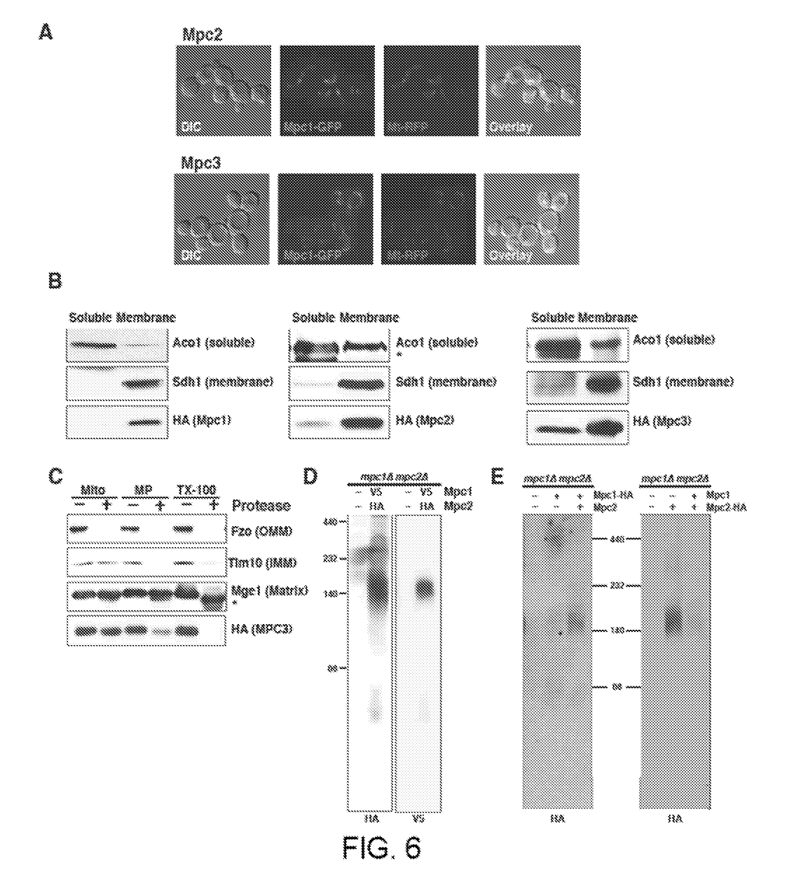

As part of an ongoing effort to characterize mitochondrial proteins that are conserved through evolution, studies of the Mitochondrial Pyruvate Carrier (MPC) protein family (originally designated BRP44 and BRP44L in humans) were initiated [Jiang et al., 2009]. This family contains three members in S. cerevisiae, encoded by YGL080W, YHR162W, and YGR243W, hereafter referred to as MPC1, MPC2 and MPC3, respectively. Mpc2 and Mpc3 are 79% identical in amino acid sequence and appear to be the product of a recent gene duplication event. Mpc1, Mpc2 and Mpc3 co-localize with mitochondria (FIG. 1A and FIG. 6A), consistent with published mitochondrial proteomic studies [Pagliarini et al., 2008; Sickmann et al., 2003]. The mitochondrial localization of Mpc1 and Mpc2 was confirmed by biochemical fractionation (FIG. 1B). Mpc1, Mpc2, and Mpc3 were enriched in mitochondrial membranes (FIG. 6B), consistent with the presence of predicted transmembrane domains in their sequences (FIG. 5). Mpc1 and Mpc2 were resistant to protease treatment unless the mitochondrial outer membrane was ruptured (FIG. 1B and S6C), implying that they are embedded in the mitochondrial inner membrane. Chromatographic purification of tagged variants of Mpc1 and Mpc2, followed by mass spectrometry, revealed that Mpc2 and Mpc3 were among the major interacting proteins of Mpc1, and that Mpc1 and Mpc3 were among the major interacting proteins of Mpc2 (Table 2). Consistent with this, immunoprecipitation of tagged Mpc1 co-purified Mpc2 and vice versa (FIG. 1C, lanes 3,4). In addition, Mpc2 interacted with itself (FIG. 1C, lane 8), while an Mpc1 homotypic interaction was not detected (FIG. 1C, lane 7). Blue native-polyacrylamide gel electrophoresis (BN-PAGE) showed that both Mpc1 and Mpc2 migrated as part of an ˜150 kilodalton (kDa) complex (FIG. 6D). Loss of Mpc2 prevented Mpc1 from migrating in this complex, while an mpc1Δ. strain showed elevated Mpc2 complex formation (FIG. 6E). It was concluded that Mpc1 and Mpc2 form a multimeric complex embedded in the mitochondrial inner membrane, with Mpc2 being the major structural subunit.

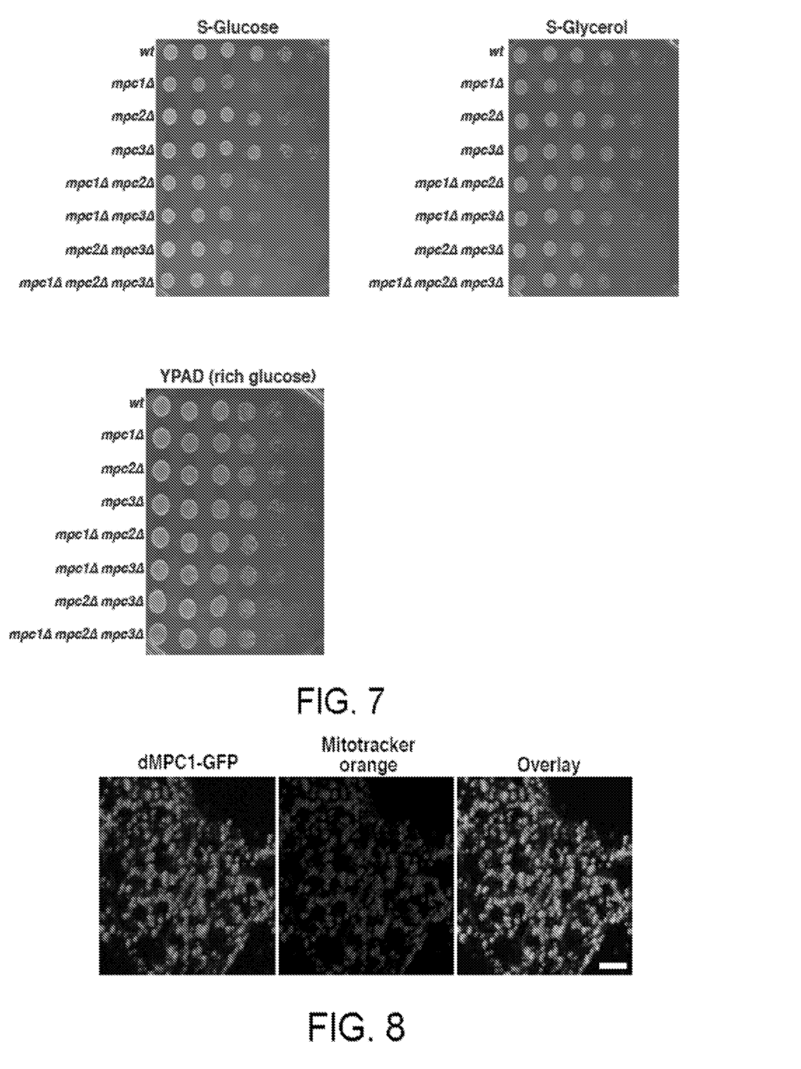

Mutant yeast strains were subjected to a variety of growth conditions. The mpc1Δ. and mpc2A. cells displayed mild growth defects on non-fermentable carbon sources like glycerol, with greater effects on glucose medium (FIG. 7) and a strong growth defect in the absence of leucine (FIG. 1D). In contrast, mpc3A. mutant displayed no apparent growth phenotypes. Yeast, Drosophila, or human MPC1 orthologs, but not human MPC2, could rescue the mpc1Δ growth phenotype (FIG. 1E), indicating that Mpc1 function is conserved through evolution.

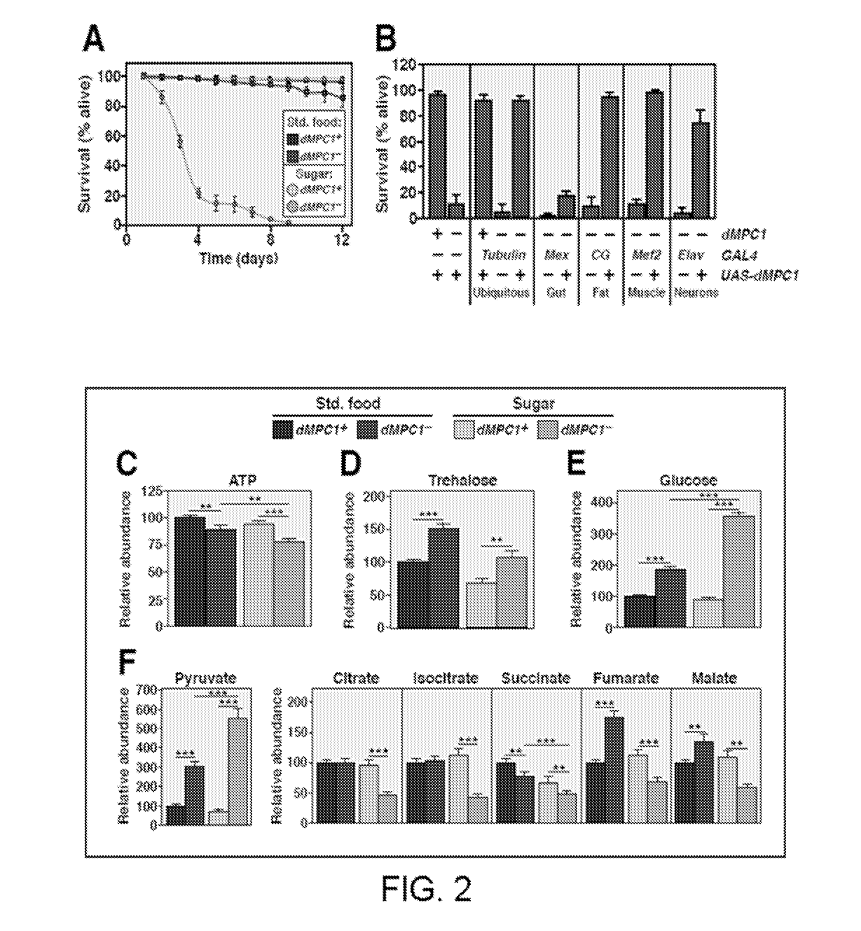

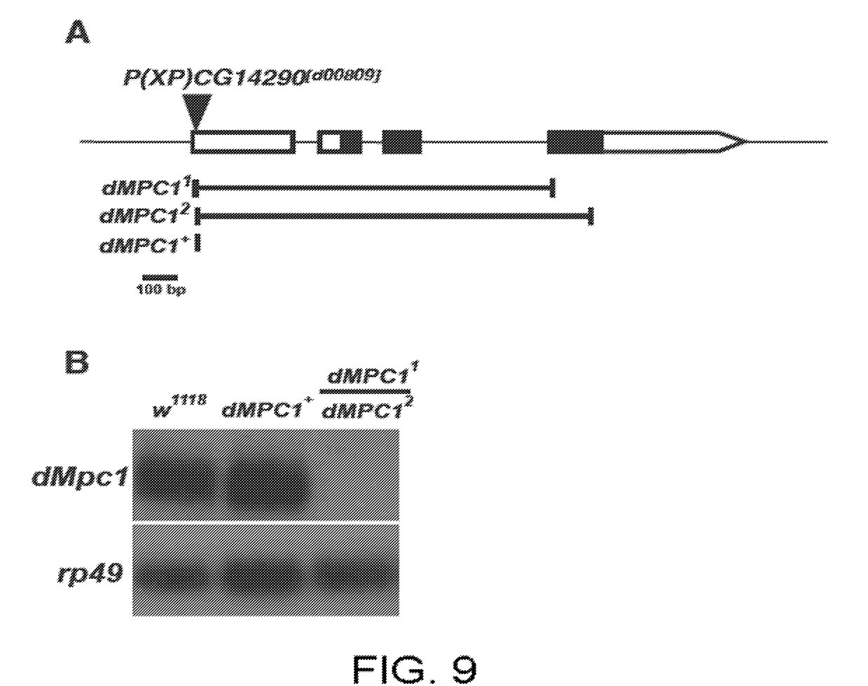

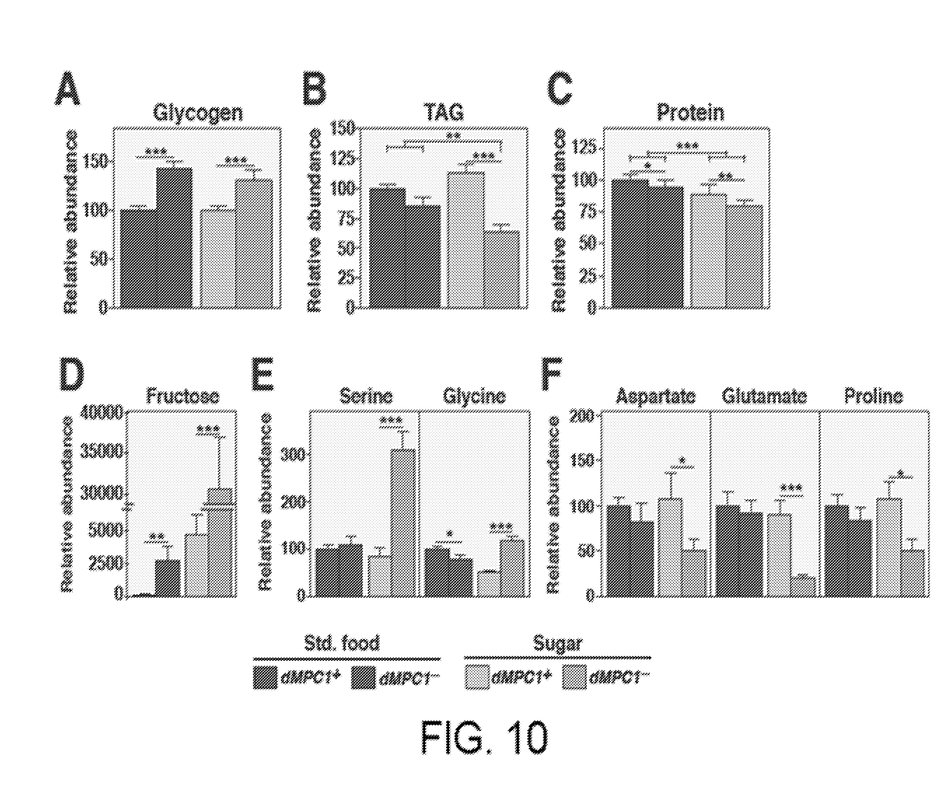

To analyze the physiological function of MPCs in a multicellular animal, the studies were extended to the Drosophila ortholog of MPC1 (dMPC1; encoded by CG14290), which also localized to mitochondria (FIG. 8). Analogous to yeast mpc1Δ mutants, dMPC1 mutants (FIG. 9) were viable on standard food, but sensitive to a carbohydrate-only diet, with rapid lethality after transfer to a sucrose medium (FIG. 2A). While ATP was reduced in dMPC1 mutants (FIG. 2C), along with TAG and protein (FIGS. 10B, and 10C), carbohydrates were elevated, including the circulating sugar trehalose (FIG. 2D), glucose (FIG. 2E), fructose, and glycogen (FIGS. 10A and 10D). These results show that dMPC1 mutants are defective in carbohydrate metabolism and may consume stored fat and protein for energy. Consistent with this, the lethality of dMPC1 mutants on the sugar diet was rescued by expression of the wild-type gene in tissues that depend heavily on glucose metabolism: the fat body, muscle, and neurons (FIG. 2B).

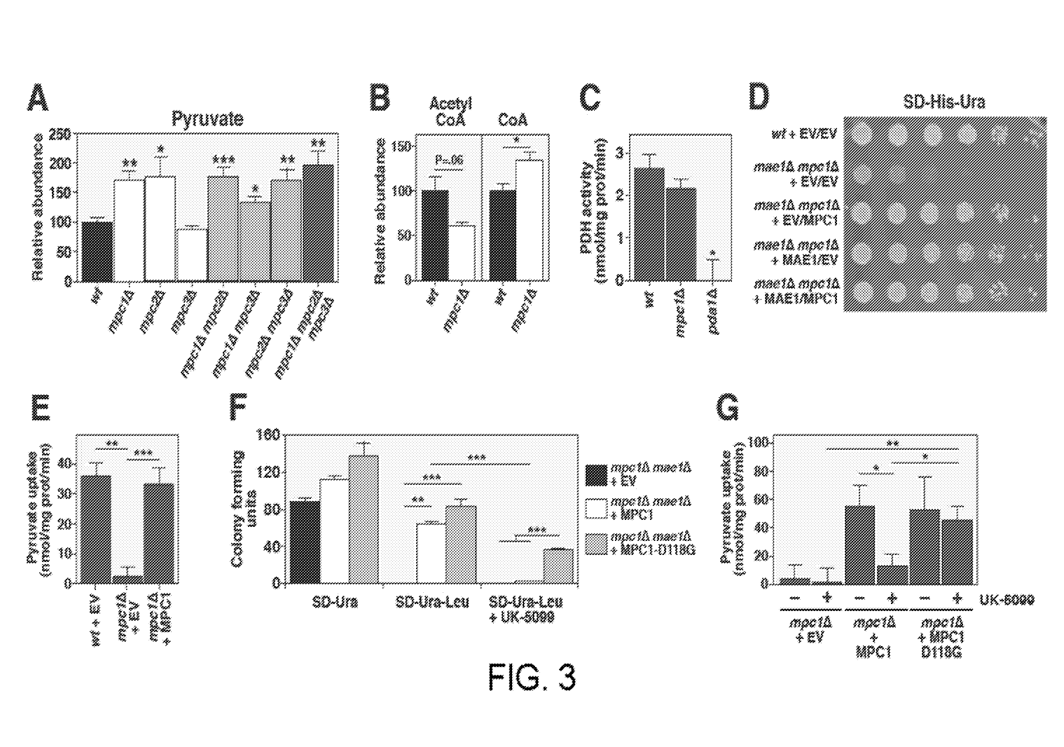

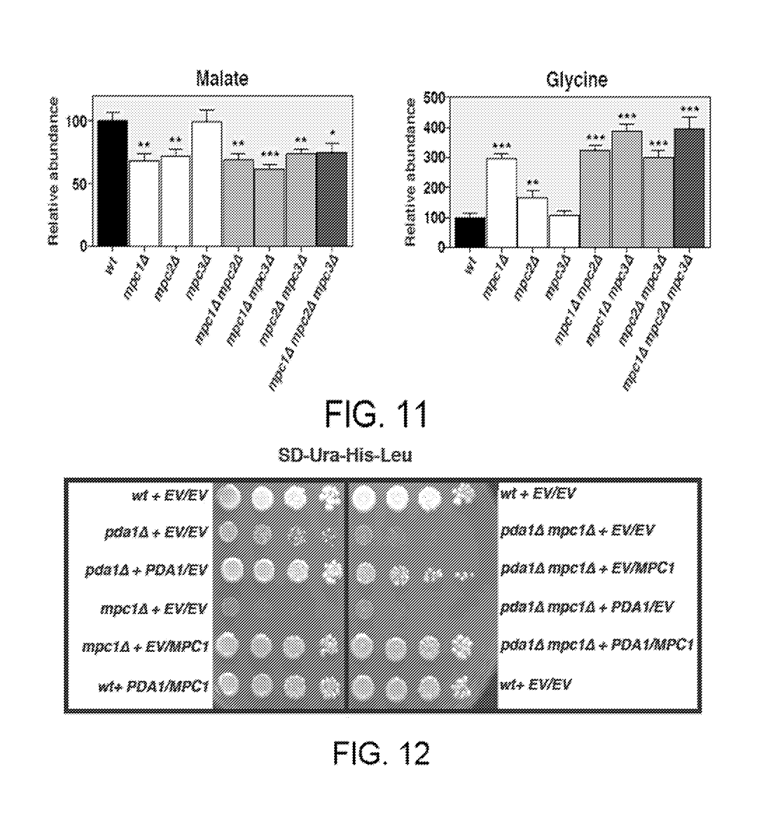

Metabolomic analyses revealed that pyruvate was highly elevated, while TCA cycle intermediates were depleted in dMPC1 mutants on the sugar diet (FIG. 2F). Similarly, glycine and serine, which can interconvert with glycolytic intermediates, were elevated in the mutants on the sugar diet (FIG. 10E), while glutamate, aspartate, and proline, which can interconvert with TCA cycle intermediates, were depleted under these conditions (FIG. 10F). Consistent with this, metabolomic analysis of mpc1Δ and mpc2Δ yeast mutants revealed elevated pyruvate levels (FIG. 3A), depletion of malate (FIG. 11), depleted acetyl-CoA, and elevated CoA levels (FIG. 3B). Taken together, these results show that MPC1 mutants are unable to efficiently convert cytosolic pyruvate to mitochondrial acetyl-CoA to drive the TCA cycle and ATP production.

These phenotypes arise from either a defect in mitochondrial pyruvate uptake or the conversion of mitochondrial pyruvate into acetyl-CoA by the pyruvate dehydrogenase (PDH) complex. Yeast lacking MPC1, however, had nearly wild-type PDH activity, unlike the strong decrease seen in pda1Δ mutants (FIG. 3C), which lacked PDH function [Steensma et al., 1990]. A decrease in PDH activity also does not explain the growth defect of mpc1Δ mutants, which is more severe than that of the pda1Δ mutant (FIG. 12). However, combining the mpc1Δ allele with a deletion for mae1, which encodes a malic enzyme that converts malate to pyruvate in the mitochondrial matrix [Boles et al., 1998], revealed a growth defect on glucose medium that was completely rescued by plasmid expression of either MAE1 or MPC1 (FIG. 3D). Importantly, mitochondria from the mpc1Δ mutant displayed almost no uptake of 14C-pyruvate, which could be fully rescued by plasmid expression of wild-type MPC1 (FIG. 3E). Moreover, Mpc1 was found to be a key target for UK-5099, which is an inhibitor of the mitochondrial pyruvate carrier [Halestrap 1975]. The mae1Δmpc1Δ double mutant displayed reduced growth on glucose medium lacking leucine, and this phenotype could be effectively rescued by transgenic expression of wild-type MPC1 in the absence, but not the presence, of UK-5099 (FIG. 3F). By screening for MPC1 mutants that could grow in the presence of UK-5099, a D118G substitution in MPC1 was recovered that conferred UK-5099 resistance (FIG. 3F). Moreover, while 14C-pyruvate uptake into mitochondria expressing wild-type MPC1 was almost completely inhibited by UK-5099, efficient pyruvate uptake that is resistant to UK-5099 was recovered upon expression of MPC1-D118G (FIG. 3G). It was thus concluded that MPC1 is a key component of the mitochondrial pyruvate carrier.

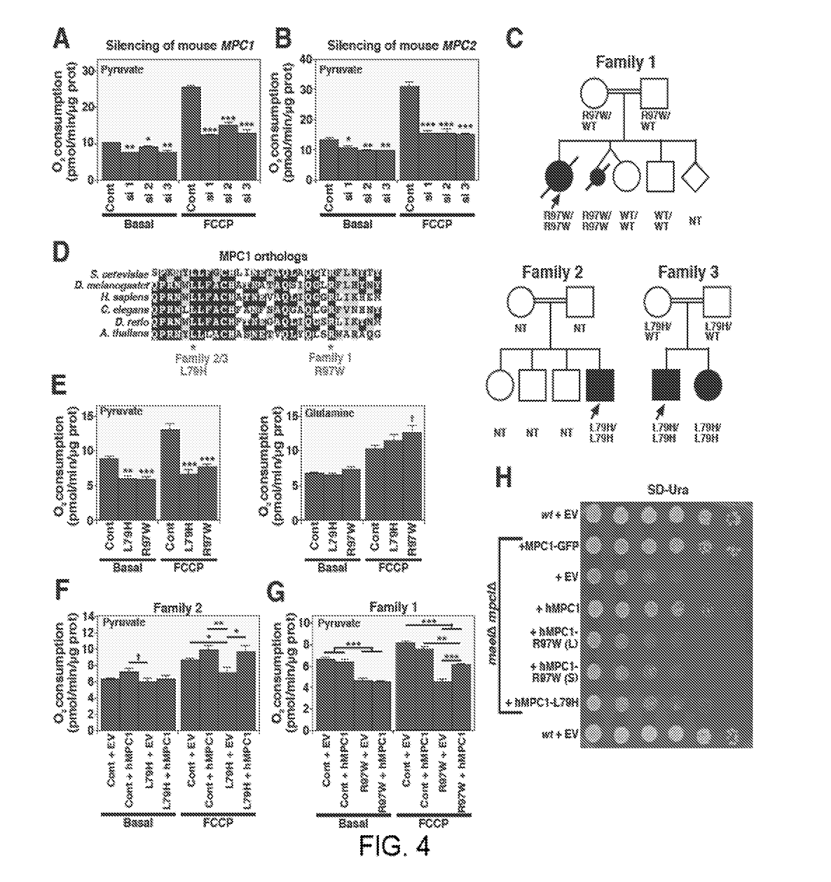

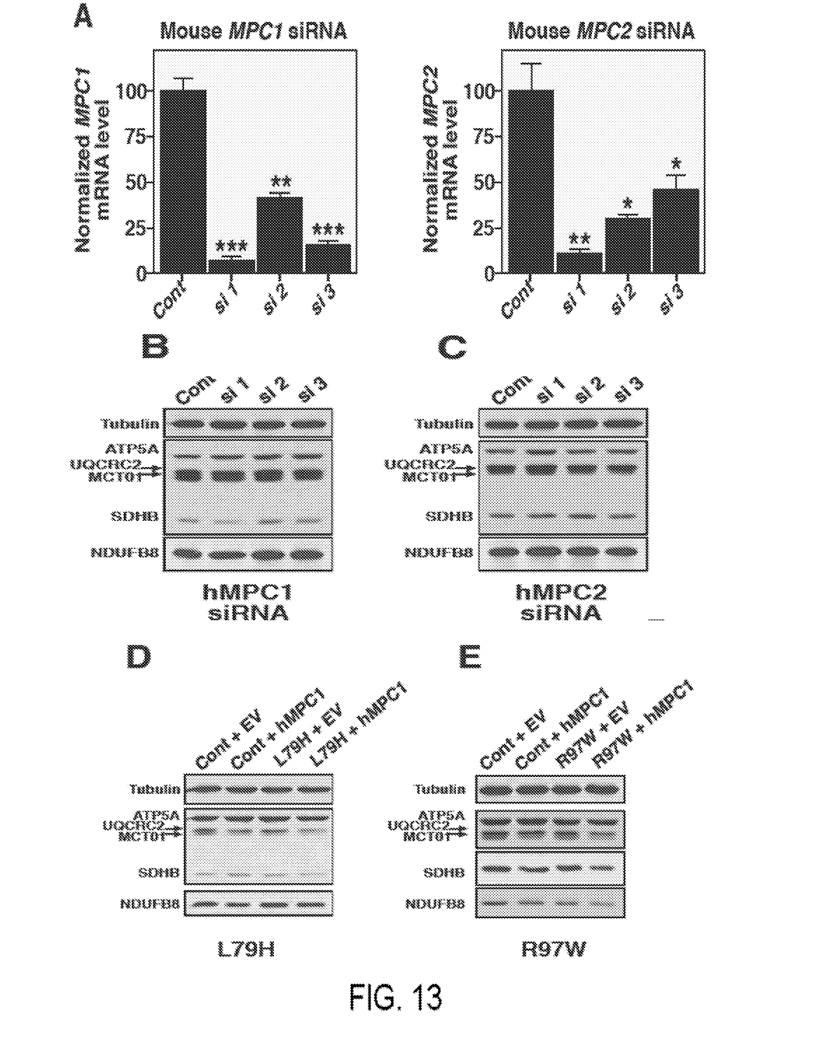

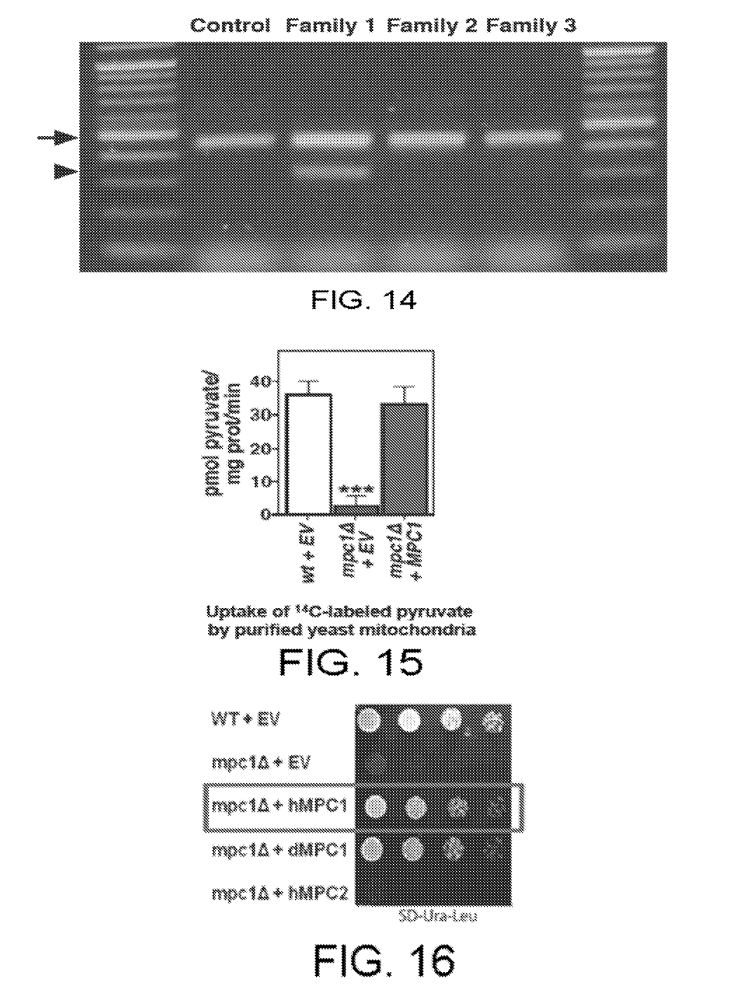

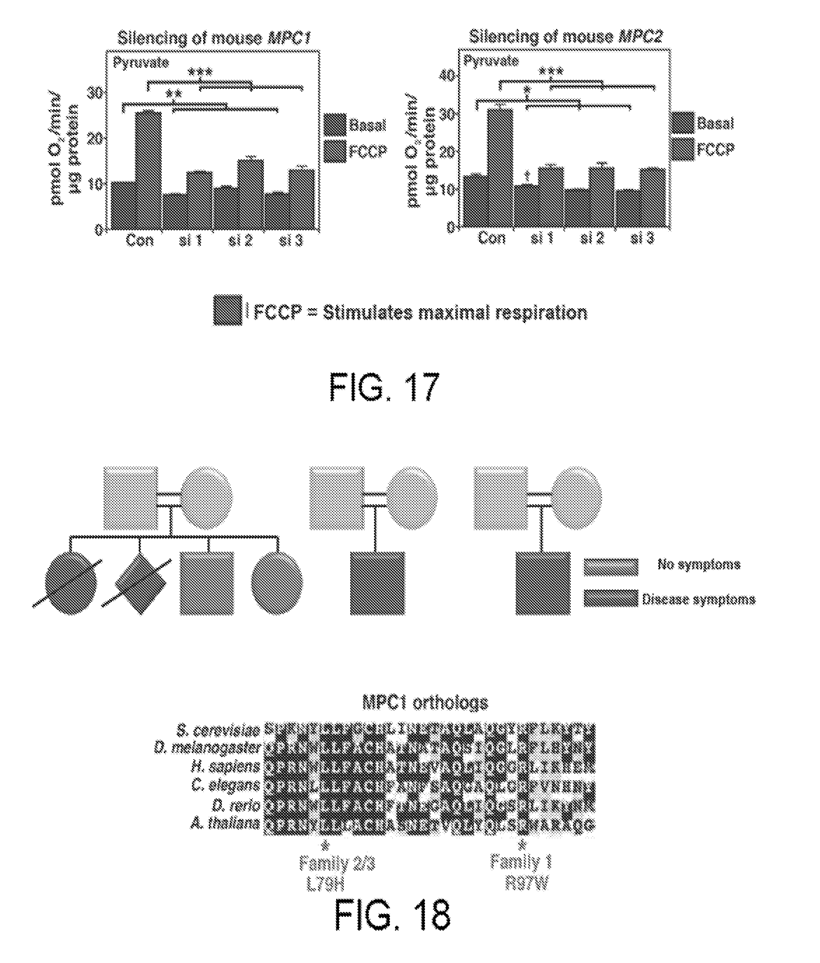

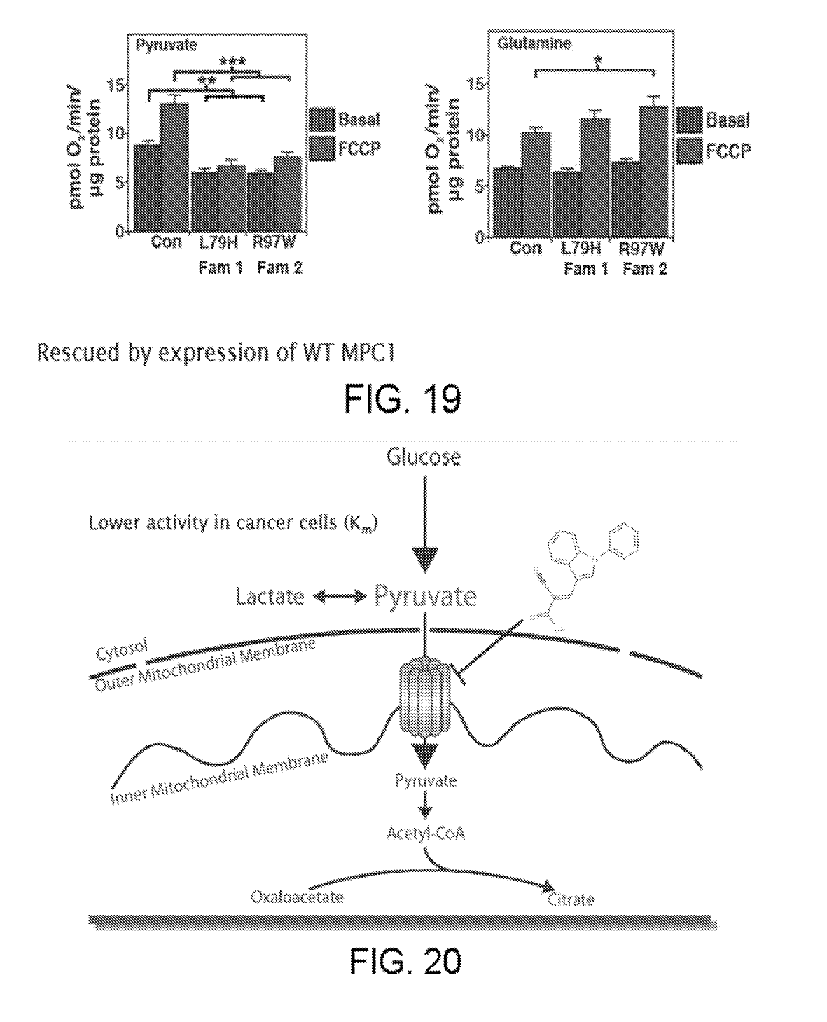

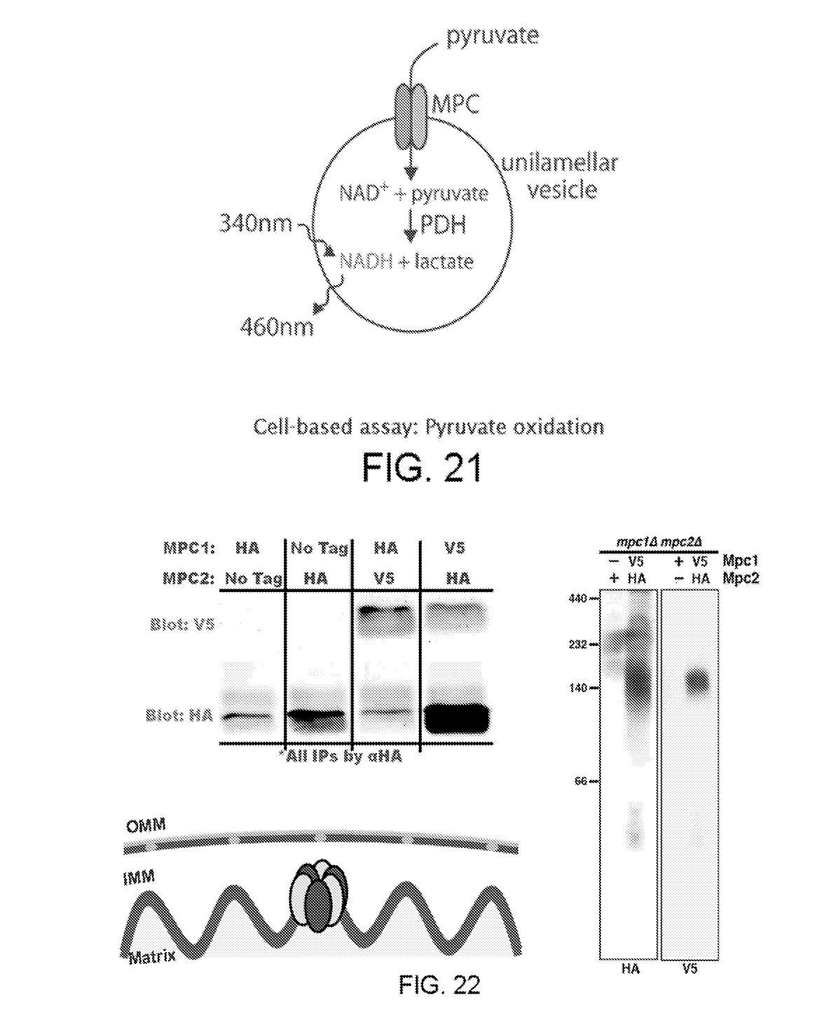

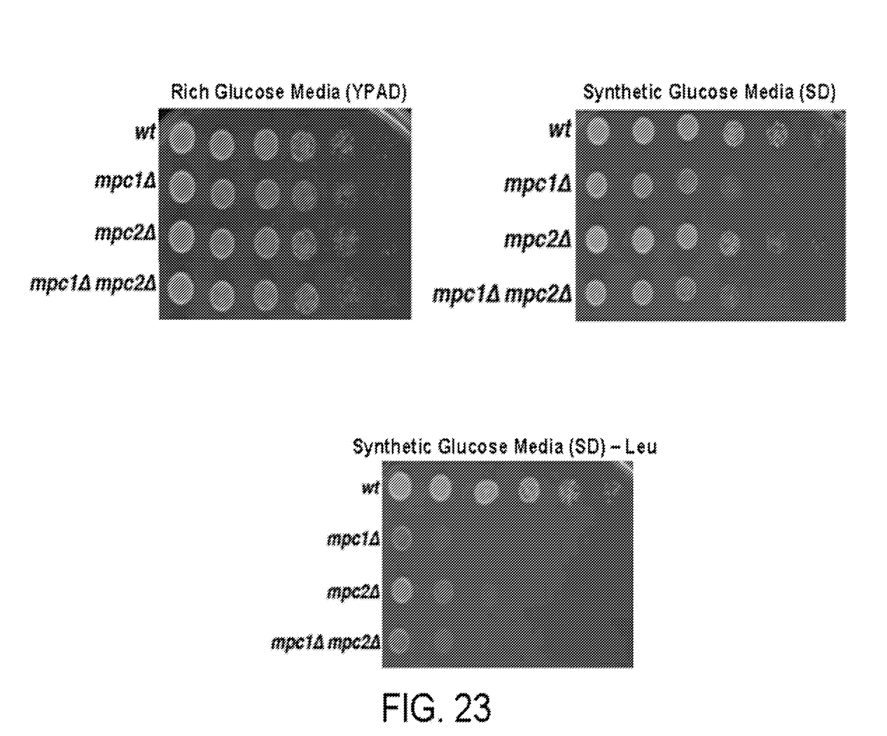

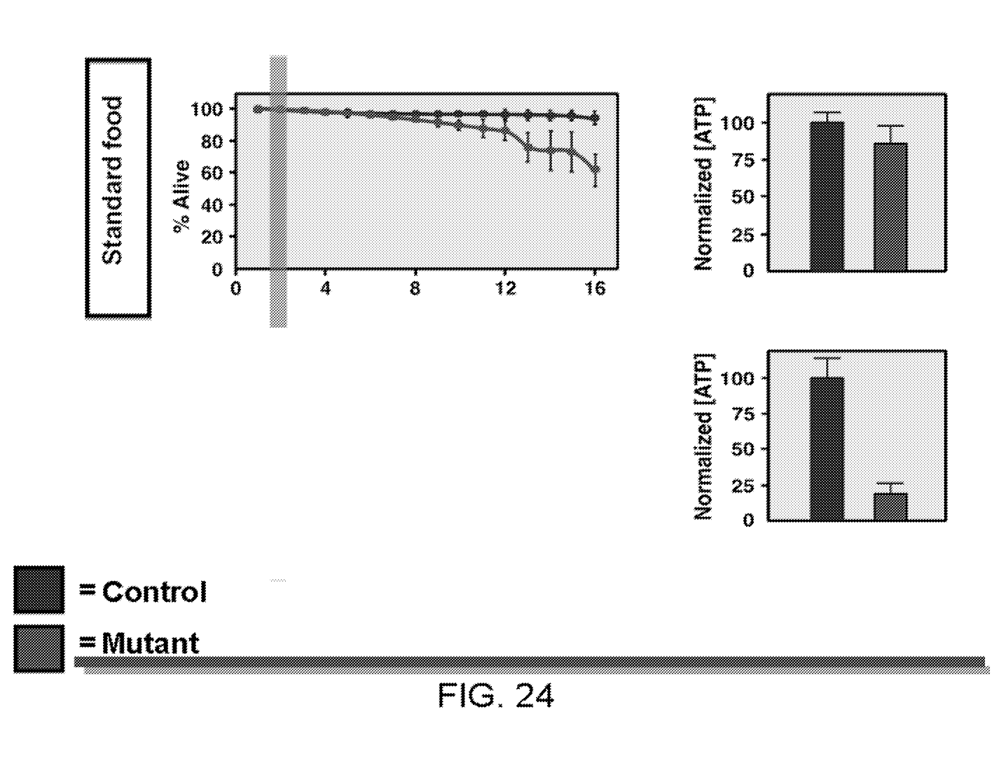

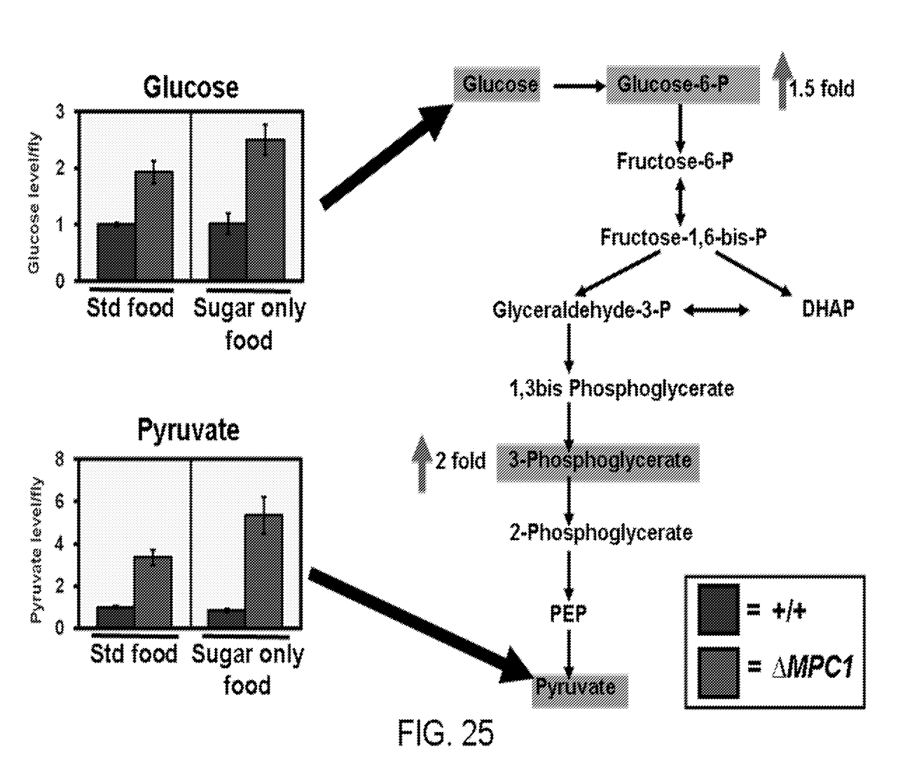

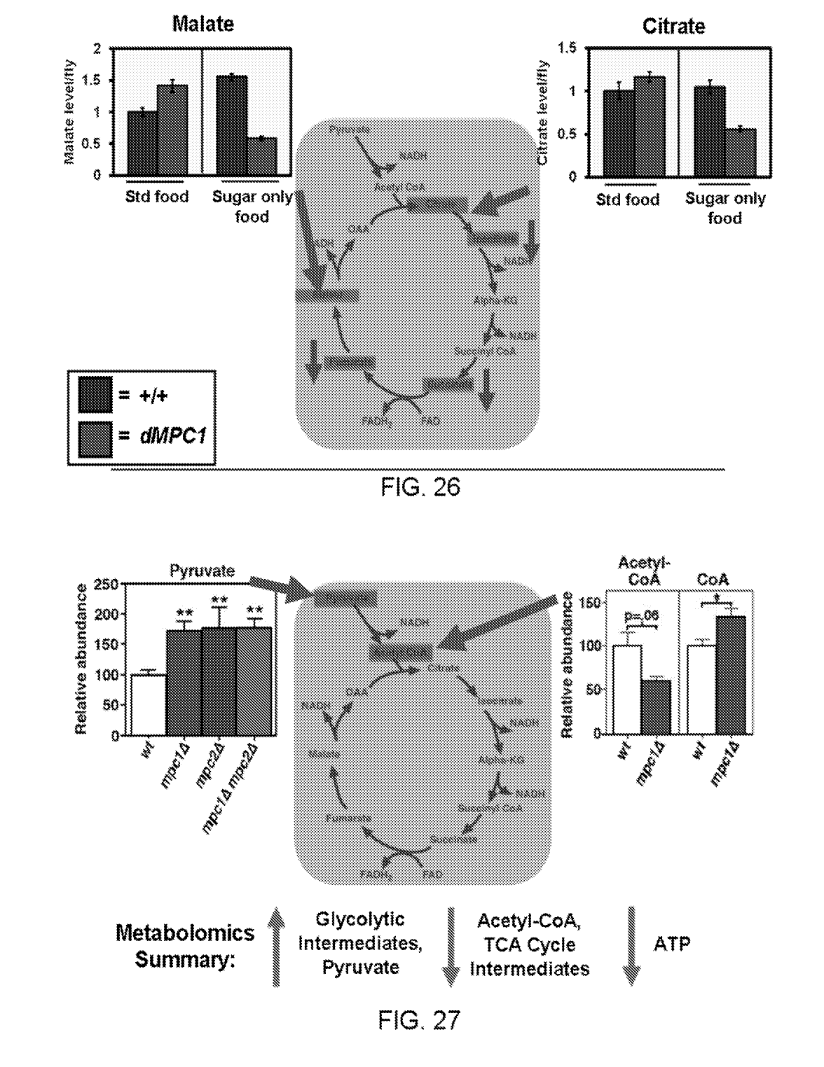

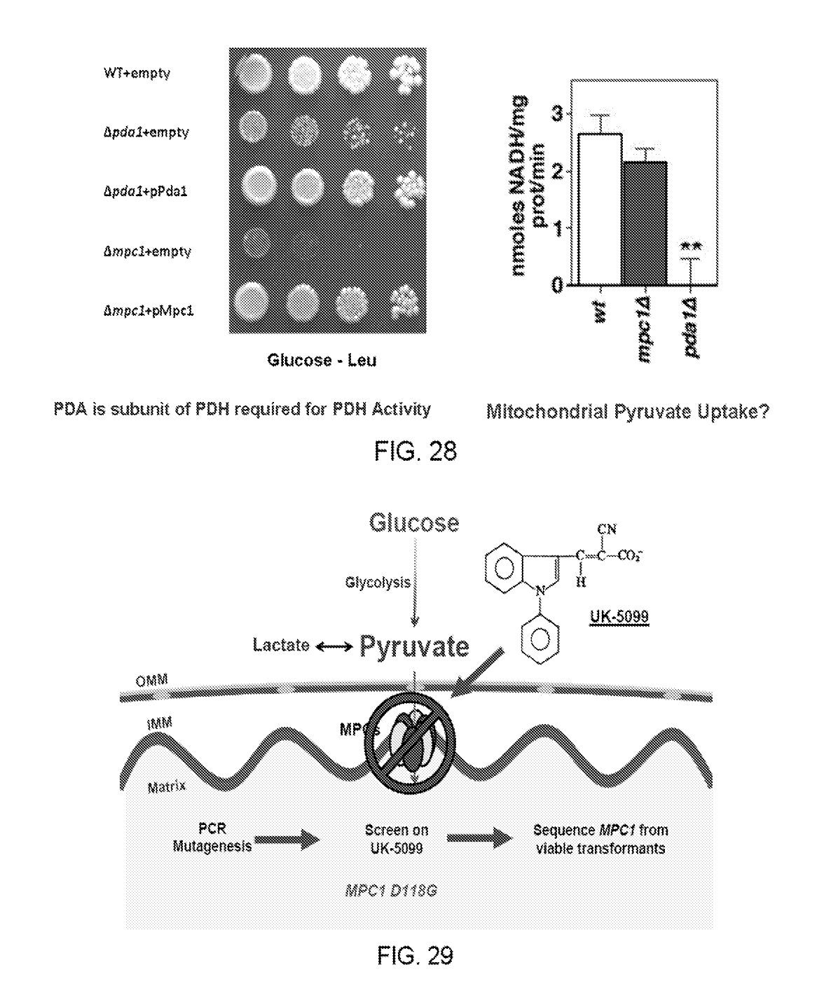

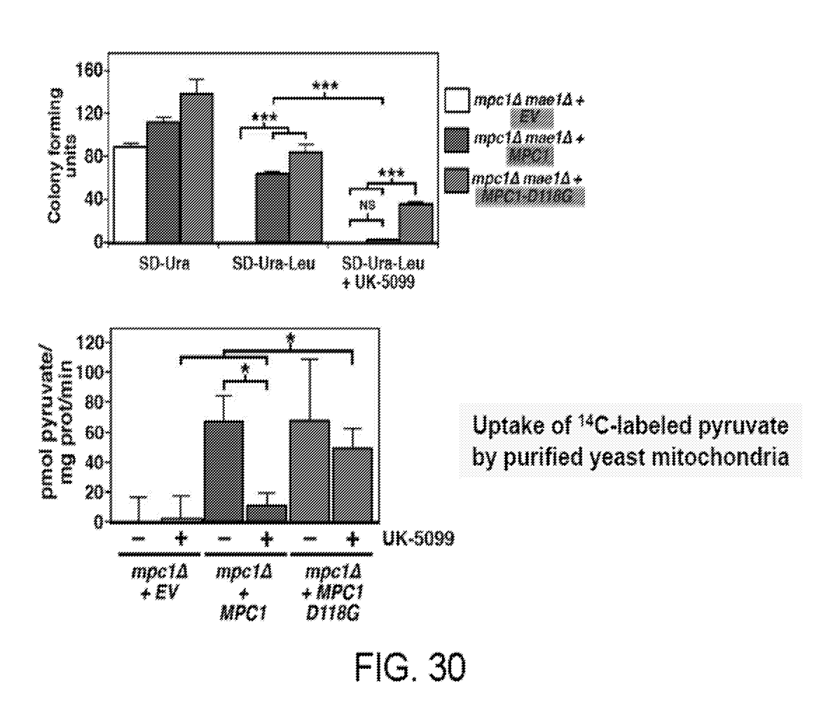

Depletion of MPC1 in mouse embryonic fibroblasts (FIG. 13) caused a modest decrease in pyruvate-driven oxygen consumption under basal conditions, and a stronger reduction in the presence of carbonyl cyanide-p-trifluoromethoxyphenylhydrazone (FCCP), which stimulates maximal respiration (FIG. 4A). Similar results were also seen upon silencing MPC2 (FIG. 4B and FIG. 13). This suppression of pyruvate oxidation, which occurred without affecting components of the oxidative phosphorylation machinery (FIGS. 13B and 13C), suggests that mammalian Mpc1 and Mpc2 mediate mitochondrial pyruvate uptake in a manner similar to that seen in yeast and Drosophila.

A French-Algerian family was previously described having two offspring that exhibited a devastating defect in mitochondrial pyruvate oxidation [Brivet et al., 2003] (FIG. 4C, Family 1). Two additional families were subsequently discovered, each with one affected child who displayed a similar, but less severe, phenotype (FIG. 4C, Families 2 and 3). Linkage analysis and homozygosity mapping allowed the study to focus on one candidate region on chromosome 6 (163,607,637-166,842,083, GRCh37/hg19). This interval contained 10 potential candidate genes: PACRG, QKI, C6orf118, PDE10A, SDIM1, T, PRR18, SFT2D1, RPS6KA2, and BRP44L, which is the human MPC1. DNA sequencing of the exons and intron/exon boundaries of the MPC1 gene in fibroblasts from the affected patients in families 2 and 3 revealed the same molecular lesion, c.236T>A, causing a predicted p.Leu79His alteration (FIG. 4D). Analysis of DNA from family 1 revealed a distinct sequence change, c.289C>T, which resulted in a predicted p.Arg97Trp mutation (FIG. 4D). Both of the affected residues are conserved through evolution between MPC1 orthologs, and Arg97 is conserved amongst both MPC1 and MPC2 orthologs (FIG. 5).

In FIG. 4D, with respect to the MPC1 orthologs, the following apply: S. cerevisiae (SEQ ID NO:19); D. melanogaster (SEQ ID NO:20); H. sapiens (SEQ ID NO:21); C. elegans (SEQ ID NO:22), D. rerio (SEQ ID NO:23); and A. thaliana (SEQ ID NO:24).

In FIG. 5, with respect to MPC1, the following apply: S. cerevisiae (SEQ ID NO:25); D. melanogaster (SEQ ID NO:26); H. sapiens (SEQ ID NO:27); C. elegans (SEQ ID NO:28), D. rerio (SEQ ID NO:29); and A. thaliana (SEQ ID NO:30). In FIG. 5, with respect to MPC2, the following apply: S. cerevisiae (2) (SEQ ID NO:31); S. cerevisiae (3) (SEQ ID NO:32); D. melanogaster (SEQ ID NO:33); H. sapiens (SEQ ID NO:34); C. elegans (SEQ ID NO:35), D. rerio (SEQ ID NO:36); and A. thaliana (SEQ ID NO:37).

Cells from the affected individuals in families 1 and 2 exhibited impaired basal and FCCP-stimulated pyruvate oxidation (FIG. 4E), while glutamine-driven oxygen consumption was normal or elevated, demonstrating that they have not acquired a generalized impairment of mitochondrial respiration (FIG. 4E). As expected, expression of wild-type human MPC1 in the cells from family 2 (FIG. 4F) or family 1 (FIG. 4G) either completely or partially rescued the defect in FCCP-induced pyruvate oxidation. Moreover, expression of the MPC1-Leu79His allele was less effective at suppressing the yeast mpc1Δ growth defect relative to wild-type human MPC1 (FIG. 4H), while the stronger MPC1-Arg97Trp allele was essentially inactive (FIG. 4H), suggesting that MPC1 function is evolutionarily conserved from yeast to man.

In summary, the data presented herein demonstrates that the Mpc1/Mpc2 complex is a component of the mitochondrial pyruvate carrier in yeast, flies and mammals. This is consistent with experiments performed in rat liver, heart, and castor beans, which implicated proteins of 12-15 kDa in mitochondrial pyruvate uptake [Thomas et al., 1981] —similar to the molecular weights of Mpc1 (15 kDa), Mpc2 (14 kDa) and Mpc3 (16 kDa). Although these individual sizes are relatively small, Mpc1 and Mpc2 form a complex of ˜150 kDa, suggesting that an oligomeric structure mediates pyruvate transport. Finally, it is important to note that the degree to which carbohydrates are imported into mitochondria and converted into acetyl-CoA is a step in normal glucose oxidation as well as the onset of diabetes, obesity and cancer. Thus, like PDH, which is controlled by allostery and post-translational modification [Harris et al., 2002], the mitochondrial import of pyruvate is likely to be a precisely regulated process [Zwiebel et al., 1982; Rognstad et al., 1983]. The identification of Mpc1 and Mpc2 as important for mitochondrial pyruvate transport provides a new framework for understanding this level of metabolic control as well as new directions for potential therapeutic intervention.

Materials and Methods:

Yeast Strains

Saccharomyces cerevisiae haploid strain BY4741 (MATa his3 leu2 met15 ura3) was used as the parental wild-type strain in the mae1Δ mpc1Δ spot tests and JRY472 (W303a MATa his3 leu2 met15 trp1 ura3) was used as the strain for all other experiments. Single deletion mutations were created in diploid strains by the standard PCR-based homologous recombination method using KanMX4 (kanamycin resistance), NatMX4 (nourseothricin resistance), or HphMX4 (hygromycin B resistance) cassettes to replace the entire gene, followed by sporulation and tetrad dissection to isolate the desired haploid knockout. Double and triple deletion mutants were created by standard crosses. Deletion strains were verified by PCR.

Yeast Plasmids

Non-tagged and tagged yeast DNA fragments with the promoter, coding, and terminator sequences were inserted into low-copy centromeric expression vectors or high-copy 2 μL expression vectors to enable endogenous level or over-expression expression. Coding sequences were tagged at the 3′ end with coding sequences for either Green Fluorescent Protein (GFP), a tandem affinity purification-tag that consist of a His6 tag and either two HA tags, or a single V5 tag. The human BRP44 and BRP44L and Drosophila CG14290 genes were inserted into pRS416 (URA3) with a GPD promoter, to create a constitutively transcribed construct. For expression of the patient-derived mutants, cDNA was used as a template to insert the patient sequences into pRS416 (URA3) with the native promoter for yeast MPC1.

Assessment of Yeast MPC1 Sub-Mitochondrial Localization

This experiment was done following a protocol adapted from Boldogh and Pon [Wittig et al., 2006]. Mitochondria harvested from the indicated strain expressing Rcfl-His₆/HA₃ were washed once with SH buffer (0.6 M sorbitol and 20 mM Hepes-KOH, pH 7.4) to remove protease inhibitors. Fifty micrograms of mitochondria was incubated in the isotonic SH buffer or hypertonic H buffer (20 mM Hepes-KOH) with and without 1% Triton X-100. To initiate protease digestion, 1 μg of Protease K was added and incubated on ice for 20-30 minutes. One microliter of 200 mM PMSF was added into the reaction mixtures to stop protease activity and lysate was denatured in Laemmli buffer and resolved on a 12% SDS-PAGE, followed by immunoblot. To assess the solubility of MPC proteins, soluble and membrane fractions of the intact mitochondria homogenized by sonication were separated by centrifugation and precipitated in 15% TCA.

Blue-Native Polyacrylamide Gel Electrophoresis (BN-PAGE)

BN-PAGE was done as described previously [Park et al., 2006]. Seventy-five μg of mitochondria harvested from the strain indicated was resuspended in lysis buffer (50 mM NaCl, 5 mM 6-aminocaproic acid, 50 mM imidazole, 1 mM AEBSF, and protease inhibitor cocktail) and solubilized in 1% digitonin. The lysate was resolved on a 8-18% gradient native PAGE gel using a PROTEAN® II xi Cell gel running system (Bio-rad). Immunoblot was performed by soaking the gel in transfer buffer (192 mM glycine, 25 mM Tris-base, 5% methanol, and 0.1% SDS) for 1 hour and transferring it onto a PVDF membrane in a Trans-Blot® transfer cell (Bio-rad). Membrane was blocked in 5% non-fat milk/TBST and probed with antibodies as indicated.

Drosophila Stocks

The transposable P-element insertion P(XP)CG14290^{[d00809]} was used to generate dMPC1 mutant alleles by imprecise excision. Two deletion alleles (dMPC1¹ and dMPC1²) and a precise excision were isolated and verified by PCR and sequencing. For all experiments, dMPC1⁻ denotes dMPC1¹/dMPC1² transheterozygotes and dMPC1⁺ denotes animals that are homozygous for the precise excision allele and thus wild-type for dMPC1 function. To generate the UAS-dMPC1 construct, the dMPC1 cDNA (BDGP Gold GH10244) was sequenced, excised by digestion with XhoI and EcoRI, and cloned into the pUAST vector. To generate the UAS-MPC1-eGFP construct, eGFP was amplified from pPelican using the primers 5′-CTCGAGGTGAGCAAGGGCGAGG-3′ (SEQ ID NO:38) and 5′-GGTACCTTACTTGTACAGCTCGTCCATG-3′ (SEQ ID NO:39), cut with XhoI and KpnI and cloned into pUAST to create pUAST-eGFP. The dMPC1 cDNA was then amplified by PCR to exclude the stop codon using primers 5′-CGGAATTCAACTTTCGGAGTGACAACACG-3′ (SEQ ID NO:40) and 5′-CGCTCGAGGGCTGCCGCCTGCTGCTCCTTAGAC-3′ (SEQ ID NO:41), cut with XhoI and EcoRI and cloned into pUAST-eGFP. All vectors used for generating transgenic lines were verified by restriction digestion and DNA sequencing. Transgenic lines were generated using standard P-element mediated transformation. All GAL4 lines were obtained from the Bloomington Stock Center. Mex-GAL4, CG-GAL4 and Elav-GAL4 transgenes on the second chromosome were crossed into the dMPC1¹ and mutant background using balanced crossing schemes. Tubulin-GAL4, Mef2-GAL4, and UAS-dMPC1 on the third chromosome were recombined onto either the dMPC1¹ or dMPC1² chromosome. The presence of dMPC1 deletion alleles in the transgenic lines was verified by PCR.

Assessment of Drosophila MPC1 Mitochondrial Localization

Fat body from adult Tubulin-GAL4/+; UAS-dMPC1-eGFP/+ animals was dissected in PBS and incubated for 30 minutes in 100 nM Mitotracker Orange CMTMROS (Invitrogen M7510). Following several washes, tissue was mounted in PBS. Imaging was performed using a Leica TCS SP2 confocal microscope using an excitation wavelength of 488 nm for eGFP and 543 nm for Mitotracker Orange CMTMROS. Emission spectra analyzed were 500-550 nm for eGFP and 550-620 nm for Mitotracker Orange CMTMROS. A Z-stack of 25 slices was imaged for each setting sequentially. The maximum intensity projection was generated in Image-J and used to generate an overlay. A 400×400 pixel region was enlarged and shown to focus on subcellular features.

Drosophila Dietary Treatments

Flies were maintained on standard Bloomington stock center medium at 25° C. in a 12:12 light:dark cycle. For diet switching experiments, animals were allowed to eclose for four days and were then transferred to standard media supplemented with yeast paste and allowed to mature for five days. Males were collected and transferred in groups of 10-20 to vials containing the same food, or to vials containing 10% sucrose, 1% agar in PBS. Lethality was assayed by counting immobile flies that were not responsive to touch. For survival assays, at least 40 flies of each genotype/dietary condition were assayed per experiment and each experiment was repeated two to three times. Representative data are shown.

Drosophila Metabolic Assays

For ATP measurements, adult male flies were homogenized in extraction buffer (6 M guanidine-HCl, 100 mM Tris, 4 mM EDTA) and boiled for 5 minutes. Homogenates were spun down and diluted for 1:750 in extraction buffer as described [Palanker et al., 2009] and ATP was measured using a luminescence-based assay (ATP Determination Kit, Invitrogen). ATP was measured three days after transfer to either standard food or the sugar diet as indicated. To measure glycogen, glucose, TAG and protein, male adult flies were homogenized in cold PBS and metabolites were measured essentially as described [Tennessen et al., 2011] except that lysates used for glycogen, glucose and TAG determination were heat treated at 70° C. for 10 minutes immediately after homogenization. Amyloglucosidase (Sigma) and a glucose assay kit (GO kit, Sigma) were used for glycogen and glucose determination. TAG was measured using Triglyceride Reagent and Free Glycerol Reagent (Sigma). Protein was measured using Bradford's reagent (Bio-Rad). For trehalose determination, male adult flies were homogenized in trehalase buffer and trehalose levels were measured as previously described [Canelas et al., 2009] using a porcine trehalase and a glucose assay kit (GO kit, Sigma). TAG, trehalose, glucose, glycogen and protein levels were measured two days after transfer to either the standard food or sugar diet as indicated. In all experiments, five flies were pooled per biological replicate and 5-6 biological replicates were assayed per condition/genotype. Combined results from at least three independent experiments are shown.

Drosophila Metabolomics Sample Preparation

For GC/MS analysis, male adult flies were transferred to either standard food or the sugar diet for two days, after which, they were washed multiple times in PBS and snap frozen in liquid nitrogen. Frozen flies were transferred to tubes containing 1.4 mm ceramic beads and 500 μL of cold (−20° C.) 90% MeOH was added and the flies pulverized using a MP Bio FastPrep 24 bead mill. The tubes were quickly transferred to −20° C. and incubated for 1 hour. Following this cell debris was removed by centrifugation. A second extraction step was performed on the pellet using cold (−20° C.) 60% MeOH. The supernatants were combined and dried en vacuou. Samples were then analyzed by GC/MS as described below. For each experiment, 15 flies were pooled per biological replicate and 5-6 biological replicates were assayed per condition/genotype. Combined results from three independent experiments are shown.

Yeast Metabolomics Sample Preparation

Starter cultures were inoculated with a single colony into either YPD for glycolytic conditions or YPE for respirative conditions and grown overnight. These cultures were used to inoculate 80 mL of SD media containing the nutrients needed to supplement the auxotrophies. The initial inoculation was OD₆₀₀ of 0.01 for YPD and 0.1 for YPE. 10 mL of this culture was transferred into eight (8) 25×150 mm culture tubes containing three 3 mm glass beads. Eight biological replicates of each strain were used per experiment. The cultures were grown to an OD₆₀₀ of 1 and harvested using the method of Canelas [Cadwell et al., 1994]. Briefly, 5 mL of culture was added to 20 mL of −40° C. MeOH followed by centrifugation at 5000×g for 3 minutes at −20° C. The supernatant was removed and the pellet gently washed with 2 mL of −40° C. 80% MeOH (aq) and centrifuged. The wash was removed and 5 mL of boiling 75% EtOH (aq) was immediately added, vortexed then incubated at 90° C. for five minutes. Cell debris was removed by centrifugation at 5000×g for three minutes. The supernatant was removed to new tubes and dried under vacuum. A total of six biological replicates per group was prepared for GC/MS analysis and four biological replicates per group was prepared for LC/MS analysis.

GC-MS Analysis

All GC-MS analysis was performed with a Waters GCT Premier mass spectrometer fitted with an Agilent 6890 gas chromatograph and a Gerstel MPS2 autosampler. Dried samples were suspended in 40 μL of a 40 mg/mL O-methoxylamine hydrochloride in pyridine and incubated for one hour at 30° C. To autosampler vials was added 20 μL of this solution with MSTFA added using the autosampler and incubation for 30 minutes at 37° C. with shaking. 1 μL of sample was injected to the inlet which was held at 250° C. The gas chromatograph an initial temperature of 95° C. for one minute followed by a 40° C./min ramp to 110° C. and a hold time of 2 minutes. This was followed by a second 5° C./min ramp to 250° C. then a third ramp to 350° C. and a final hold time of 3 minutes. A 30 m Restek Rxi-5 MS column with a 5 m long guard column was employed for analysis. Data was collected by MassLynx 4.1. Data analysis for known metabolites was performed using QuanLynx. To find possible unknown metabolites MarkerLynx was used for peak picking and this data was exported to SIMCA-P ver. 12.0.1 where PCA and PLS-DA analysis was performed. All data was saved to an Excel spread sheet for further analysis.

LC-MS Analysis

Samples were analyzed using a Phenomenex (Torrence, Calif.) 3.0 mm×150 mm Gemini-NX C18 (5 μm) column with a Phenomenex Security Guard column filled with the same packing material. The chromatographic system consisted of an integrated Shimadzu HPLC system consisting of two LC-10AD pumps, column oven and a CBM-20A 82 controller. A PE200 autosampler with a cooling unit set to 4° C. was used for sample handling. A PE Sciex API 365 mass spectrometer modified with an Ionics EP 10+ source was used for analyte detection. A mobile phase consisting of solvent A (water with 15 mM ammonium formate/6.5 mM N-dibutylamine) and solvent B (methanol/6.5 mM N-dibutylamine) was used for elution of samples. The initial condition was 5% B with an initial hold time of 3 minutes followed by a ramp to 73% B over 21 min. A second ramp to 90% B was employed over the next minute with a 1 minute hold. The column was brought back to 5% B over two minutes and re-equilibrated for 9 minutes. The flow rate was 0.3 mL/min at 24° C. Mass spectrometer transition optimization was performed using a syringe pump. For each metabolite optimized it was dissolved in buffer A as a 1 mg/mL solution. Infusion was performed at 20 μL/min while 10% B/90% A buffer was co-infused using the HPLC at 0.3 mL/min. Samples were prepared as follows: to prevent the exogenous oxidation of GSH it was derivatized using 2-vinyl pyridine. To each sample was added 48 μL of 10 mM K₂PO₄ pH 7 and 2 μL of 2-VP. A brief sonication using a water bath was performed to fully elute each sample. After 30 minutes of incubation at room temperature 50 μL of buffer A was added followed by 10 min of centrifugation at 20000×g. 90 μL of this was transferred to an autosampler vial and immediately transferred to the autosampler which was held at 4° C. until analysis. After analysis each metabolites peak height was recorded in EXCEL.

Mitochondrial Isolation

Yeast mitochondria were isolated using a simplified version of the method described by Boldogh and Pon [Wittig et al., 2006]. Briefly, yeast were grown in synthetic 2% raffinose media to an A₆₀₀˜3.0 and collected. The cells were pelleted, washed with sterile water, re-pelleted, and then frozen at −80° C. Mitochondrial isolation began by thawing the cells on ice, digesting the cell wall with lyticase (Sigma) for one hour to generate spheroplasts. Following 40 minutes in regeneration buffer, spheroplasts were lysed using a Dounce homogenizer. The mitochondria were further purified through several high and low speed centrifugation steps.

Pyruvate Uptake

Mitochondria used for pyruvate uptake experiments were extracted from live cells, kept on ice and used within 5 hours of harvest without freezing. An estimate of mitochondrial concentration was calculated using A₂₈₀. Samples were then diluted to yield equal mitochondrial mass and centrifuged at 10000 rpm for 10 minutes to pellet mitochondria. The supernatant was then removed and the mitochondrial pellet was resuspended in respiratory buffer (120 mM KCl, 5 mM KH₂PO₄, 1 mM EGTA, and 3 mM HEPES pH 7.4) and placed on ice.

To initiate pyruvate uptake, 50 μL of mitochondria was added to 100 μL of room temperature respiratory buffer at pH 6.8 in a microcentrifuge tube. The final concentration of the reaction mixture included 0.05 mM unlabeled malate along with 0.1 mM labeled pyruvic acid mixture (0.01 mM ¹⁴C-pyruvate and 0.09 mM unlabeled pyruvate). The reaction was carried out at room temperature with constant stirring using a micro stir bar. Immediately following the addition of mitochondria to the reaction buffer the timer was started and 50 μL samples were taken and spotted onto binderless glass fiber filters (0.5 to 0.7 μm pore size) at 1, 2, or 3 min time points. The filter was then rinsed with 3 mL of ice cold TBS using an aspirator to draw the wash buffer through the filter and dry it. The filter was then placed in a scintillation vial containing Ultima Gold MV for liquid scintillation counting. Blank samples in which the 50 μL of mitochondria was replaced with the buffer used to resuspend the mitochondria were taken at corresponding time points and these blank values were subtracted from the experimental data. Five biological replicates were analyzed per group.

Drug Inhibitor Studies

Pyruvate uptake was carried out as above with the following modifications. Prior to initiating the reaction, individual mitochondrial samples were incubated in 0.2 mM UK-5099 or DMSO (vehicle) for 2 min at room temperature with constant stirring. Following incubation, 100 μL of buffer containing radiolabeled pyruvate was added and the timer was started and samples were collected as described above. Results shown are combined from four independent experiments, and a total of 17-21 biological replicates per group.

Screen for UK-5099-Resistant Mutants

A plasmid library harboring MPC1 mutants was generated by PCR mutagenesis using methods previously described [Hinman et al., 1981]. mpc1Δ mpc3Δ mae1Δ triple mutant yeast cells were transformed with the MPC1 mutant library and plated onto SD-Ura-Leu supplemented 500 μM UK-5099. Plates were incubated at 30° C. for 72 hours and mutant plasmids were isolated from viable colonies, amplified in E. coli., and then transformed into naive mpc1Δmpc3Δmae1Δ triple mutant yeast cells to confirm the conferral of resistance to UK-5099. An MPC1 mutant containing a D118G substitution was recovered from this screen. To quantify UK-5099 resistance conferred by the D118G substitution, uniform suspensions of mpc1Δmpc3Δmae1Δ triple mutant yeast cells were transformed with 2 μg empty vector, plasmid harboring wild-type MPC1, or plasmid harboring MPC1 D118G, and then plated onto either SD-Ura, SD-Ura-Leu, or SD-Ura-Leu plus 500 μM UK-5099.

Immunoprecipitation

JRY472 mpc1Δ mpc2Δ was transformed with plasmids expressing tagged proteins (MPC1-HA, MPC1-V5, MPC2-HA, and MPC2-V5) or untagged MPC1 or MPC2 as controls. These strains were then grown to log phase in selective synthetic media containing 2% raffinose. Following harvest, mitochondria were isolated as above and stored at −80° C. For immunoprecipitation, 2 mg of total mitochondria was solubilized in 500 μL XWA buffer containing 150 mM NaCl, 0.8% digitonin, and protease and phosphatase inhibitors for one hour at 4° C. with gentle agitation. The debris was then pelleted and the supernatant was saved, one aliquot for input and the remainder being used for the IP. 20 μL/10 mg of HA-Agarose beads were rinsed and incubated with the supernatant from above at 4° C. with gentle agitation for 2 hours. The HA-agarose beads were then washed 5 times with buffer as above with 0.1% digitonin. Following the final wash the supernatant was aspirated and the beads resuspended in 1×SDS loading buffer and incubated at 95° C. for 10 minutes. 60 μL was then loaded to a 15% SDS polyacrylamide gel and separated by electrophoresis. Following transfer to a nitrocellulose membrane anti-HA and anti-V5 primary antibody were used in combination with fluorescent anti-mouse and anti-rabbit secondary antibodies respectively. Protein bands were visualized using an Odyssey imager (Licor Biosciences).

Pyruvate Dehydrogenase Activity Assay

The indicated strains were grown to stationary phase at 30° C. in selective media containing 2% raffinose. Once in stationary growth phase the strains were harvested and pelleted at 3000×g for five minutes and washed once with sterile water. Following resuspension, concentration was determined by A₆₀₀ and used to dilute to an OD of 1.0 in 1 liter of synthetic media containing 2% glucose. After 3 hours of growth the strains were harvested and mitochondria were isolated as described above. Mitochondria were diluted to 4 mg/mL and sonicated on ice at an amplitude of 1.0; 3 seconds on and 9 seconds off for a total sonication time of 60 seconds. Debris was pelleted by centrifugation at 10,000 rpm for 1 minute. Sonicated mitochondrial samples were then subjected to a coupled enzymatic assay with a spectrometric read out modified from the assay explained by Hinman and Blass [Brivet et al., 2003]. Briefly, purified sonicated mitochondria was used to start the pyruvate dehydrogenase reaction in 50 mM potassium phosphate buffer pH 7.8, 0.2 mM thiamine diphosphate, 2.5 mM NAD⁺, 0.1 mM CoA, 1 mM MgCl₂, 0.3 mM dithiothreitol, 5 mM pyruvate, with coupling molecules 0.6 mM 2(p-iodophenyl)-3-p-nitrophenyl-5-phenyl-tetrazolium chloride, and 6.5 μM phenazine methosulfate. The reaction proceeded with intermittent shaking at 25° C. and absorbance readings at 495 nm are taken every 30 seconds over 30 minutes. Rates are determined by calculating the change in absorbance in the linear range per unit of time. Pyruvate dehydrogenase activity is calculated by subtracting the rate of absorbance change in samples supplied with 5 mM pyruvate from identical samples lacking pyruvate. The molar absorption of INT (12.4 mM⁻¹ cm⁻¹) [Brivet et al., 2007] was used to calculate the amount of NADH in nanomoles per milligram of protein per minute. Three biological replicates were analyzed per group.

Fluorescence Colocalization

The indicated strains were transformed with a vector for expression of a C-terminally GFP-tagged Mpc1, Mpc2 or Mpc3 shown to rescue the relevant phenotype along with a Mito-RFP plasmid. These cells were grown in selective synthetic media with 2% glucose to an A₆₀₀=1.0. The cells were then observed using a Zeiss Axioplan 2 Imaging microscope (Carl Zeiss).

Mammalian Cell Culture

Mouse embryonic fibroblasts (MEFs) were isolated on day-13 postcoitum. MEFs were transformed by viral transduction with SV40 Large-T antigen (pLNX SV-40) and selection with hygromycin (200 μg/mL). Transformed MEFs were maintained in DMEM with 10% FBS, 2 mM glutamax, and 1% primocin (Invivogen). Human skin fibroblasts (HSFs) were isolated as previously described [Stoetzel et al., 2007] and immortalized by viral transduction with hTERT (Addgene Plasmid 1773) and selection with hygromycin (20 μg/mL). For rescue experiments, HSFs were reconstituted with empty vector or MPC1 by viral transduction, and selection with puromycin (1 μg/mL). HSFs were maintained in DMEM with 15% FBS, 2 mM glutamax, and 1% primocin. All viral transductions were performed with pseudotyped retroviral supernatants generated by co-transfection of 293T cells with Vsv-G, Gag-Pol, and a retroviral targeting vector harboring cDNA for the gene of interest. MPC1 cDNA was cloned into (Not1/BamHI) and delivered by the pQCXIP retroviral vector (Clontech). Knockdown of MPC1 and MPC2 was performed by treating cells with 20 nM siRNA using the Lipofectamine RNAiMax transfection reagent, according to the manufacturer's instructions (Invitrogen). The All-Stars non-targeting siRNA (Qiagen) was used as the control for siRNAs targeting MPC1 and MPC2, which were designed with the Dharmacon siDesign Center tool (http://www.dharmacon.com/designcenter/DesignCenterPage.aspx). Sequences of the sense strands of targeting siRNAs, which include a 3′ tt DNA overhang are as follows. MPC1:[Tennessen et al., 2011] UCAACUACGAGAUGAGUAAtt (SEQ ID NO:42), (2) GGGAAAACACAGAAUGCUAtt (SEQ ID NO:43), and (3) CCAUGUAACAAACGAAGUAtt (SEQ ID NO:44). MPC2: (1) CCGAUAAGGUGAUGCUAAAtt (SEQ ID NO:45), (2) UGGAUAAAGUGGAGUUGUUtt (SEQ ID NO:46), and (3) ACCAAGAACUCAAAUCUAAtt (SEQ ID NO:47). Cells were subjected to knockdown on day zero, again on day 3, and analyzed on day 6.

Measurements of Mammalian Cellular Respiration

Oxygen consumption of MEFs and HSFs was measured with the XF-24 Seahorse Bioanalyzer. The day prior to assays, cells were seeded at densities between 20-30 k (MEFs) and 15-25K (HSFs) per well of Seahorse XF-24 plates. At the time of seeding, 12-well plates were seeded proportionally to 1) enable normalization of oxygen consumption data, and 2) provide protein lysates for analysis by Western Blot. One hour prior to assays, cells were incubated, after 1 wash, with DMEM without glucose, bicarbonate, or phenol red (Sigma, D5030), but with either pyruvate at 20 mM (MEFs) or 2 mM (HSFs), or glutamine at 2 mM (HSFs). Basal Oxygen consumption was measured in the incubation media. FCCP-stimulated respiration was measured after injection of FCCP to a final concentration of either 0.5 μM (MEFs) or 1.0 μM (HSFs). For both basal and FCCP-stimulated respiration, a measurement loop was repeated 3 times: 1-minute mixing, 2 minutes waiting, and 3 minutes measuring oxygen consumption. Oxygen consumption data was normalized to total protein. Total protein values were obtained by measuring the protein content, by BCA, of the proportionally seeded wells within 12-well plates, and multiplying by the fraction of cells loaded per well in the Seahorse XF-24 plates. 5-11 biological replicates were analyzed per group in each experiment.

Human Genetic Mapping

A severe defect of mitochondrial pyruvate oxidation has been found previously in three unrelated consanguineous families of Algerian descent. Key features of the index case in family 1 have been previously reported [Stoetzel et al., 2007]. This female patient developed a severe neonatal encephalopathy with lactic acidosis and died at 19 months after progressive neurological deterioration. Pyruvate dehydrogenase deficiency was ruled out as in other cases of families 2 and 3, less severely affected and presenting essentially with psychomotor retardation without regression and epilepsy in family 2 or peripheral neuropathy in family 3. Currently these patients are alive aged 5-14 years.

Samples were subjected to a whole genome scan using The GeneChip Human Mapping 250k NspI arrays (Affymetrix, Santa Clara, Calif., USA). Homozygosity regions were defined when more than 35 consecutive SNPs were homozygous, using HomoSNP software {http://bips.u-strasbg.fr/HomoSNP/, updated from [Gudbjartsson et al., 2000}. Candidate intervals were selected when homozygous regions were observed for all affected individuals but not for unaffected ones. Homozygous haplotypes were then checked to be either identical for all 4 affected individuals (hypothesis of a common shared mutation), or identical for both affected individuals of family 1 on one side and identical for the 2 more mildly affected patients from families 2 and 3 on the other side (hypothesis of one mild and one more severe mutation). Parametric multipoint linkage analysis was performed in family 1 using software ALLEGRO 1.2 via the easyLINKAGE Plus V5.02 interface, and assuming autosomal recessive inheritance, full penetrance and a disease allelic frequency of 0.0001.

Homozygosity mapping using the three affected families allowed the identification of four candidate regions, each containing more than 35 consecutive homozygous SNPs in all four affected individuals, but not in unaffected probands from family 1. Haplotype-coherence checking permitted us to discard two regions. Out of the two remaining candidate regions, the one located on chromosome 7 was 400 kb in length (84,106,464-84,587,114, GRCh37/hg19) and supported the initial hypothesis that a common mutation was shared by all four affected individuals since all homozygous haplotypes were identical. This region contained a single gene (SEMA3A), which did not appear to be a functionally relevant candidate. The second region was located on chromosome 6 and of total length 3.2 Mbp (163,607,637-166,842,083, GRCh37/hg19). Haplotypes therein suggested the existence of two different mutations at the same locus, one carried by the more severely affected patients from family 1 and the other by the more mildly affected patients from families 2 and 3. An independent multipoint analysis on family 1 highlighted five regions of linkage reaching the maximal expected LOD score of 2.05 (taking into account 1st cousin consanguinity), one of them encompassing the entire homozygosity region on chromosome 6. While the LOD score of 2.05 is lower than required for definitive mapping to a single locus, it is maximal LOD score given the structure of Family 1 that has confirmed first cousin consanguinity. 5 other regions in family 1 showed the same LOD score, but the candidate region on chromosome 6 was the only one consistent with homozygosity mapping in the two other families. This interval on chromosome 6 contained 10 potential candidate genes: PACRG, QKI, C6orf118, PDE10A, SDIM1, T, PRR18, SFT2D1, RPS6KA2, as well as BRP44L, which encodes human MPC1.

Human MPC1 Alleles

The c.289C>T mutation in MPC1 in family 1, which caused the predicted p.Arg97Trp substitution, also caused an altered splicing pattern of the pre-mRNA, leading to the formation of a truncated mRNA that lacked exon 4 in mutant fibroblasts (FIG. 14). Sequence analysis of the other probands from family 1 revealed that the two parents were heterozygous for the c.289C>T mutation and the two unaffected children did not carry the mutation (FIG. 4C). Similarly, the two affected children in family 3 were both homozygous for the c.236T>A mutation, which caused the predicted p.Leu79His substitution, and the two parents were both heterozygous (FIG. 4C). Sequence analysis of MPC1 from 80 unaffected donors revealed no evidence of mutations in this gene. Further, the c.236T>A mutation has not been observed in the more than 5200 individuals (European Americans and African Americans) whose exome data are reported in the exome variant server database.

Primers for Amplification of Aspects of the MPC1 and MPC2 Genes

The following primers were used to amplify aspects of the MPC1 and MPC2 genes. Amplification of MPC1 exons and MPC1 cDNAs and ORFs was performed with the Hot-Start Phusion Polymerase (Thermo-Fisher) with the inclusion of Betaine to a final concentration of 1 M, and all with annealing temperatures of 72° C. Primers for the MPC1 ORF include NotI (5′) and EcoR1 (3′) restriction endonuclease sites that were utilized for cloning. Quantitative PCR (qPCR) was performed with a Roche 480 Light cycler using the Applied BioSystems Sybr Green Master Mix.

TABLE 1

| Amplicon | Primer | Sequence |
|---|---|---|
| hsMPC1 | FWD | CCAGCCCCAGCCGTTTTACGGCAG |
| Exon 1 | | [SEQ ID NO: 1] |
| hsMPC1 | REV | CTGAAAGGCGCCCACTGTCACCG |
| Exon 1 | | [SEQ NO: 2] |
| hsMPC1 | FWD | GGCACAAACCACCCATGCCCAGC |
| Exon 2 | | [SEQ ID NO: 3] |
| hsMPC1 | REV | GCTGTCACAGAGCTCGTGTCTAGGC |
| Exon 2 | | [SEQ ID NO: 4] |
| hsMPC1 | FWD | GTGTCTGGGGTCCTGGGCATTGATTTC |
| Exon 3 | | [SEQ ID NO: 5] |
| hsMPC1 | REV | GCAAGCAGGAGCTCTACTATGTTGAAAGTCC |
| Exon 3 | | [SEQ ID NO: 6] |
| hsMPC1 | FWD | CAGCCTACTCTTGCTCGGAAATATGTTCC |
| Exon 4 | | [SEQ ID NO: 7] |
| hsMPC1 | REV | CCAGTCCCGCAGCACTCCCTC |
| Exon 4 | | [SEQ ID NO: 8] |
| hsMPC1 | FWD | GCCGGGGTGTCATTGGCTCT |
| cDNA | | [SEQ ID NO: 9] |
| hsMPC1 | REV | GTGACTCAGCAGCAGCTGGCAAT |
| cDNA | | [SEQ ID NO: 10] |
| hsMPC1 | FWD | GATCGCGGCCGCACCATGGCGGGCGCGTTG |
| ORF | | [SEQ ID NO: 11] |
| hsMPC1 | REV | GATCGGATCCTTATGCAGATGCCGTTTTAGT |
| ORF | | CATCTC[SEQ ID NO: 12] |
| mmMPC1 | FWD | GCACGGCCATGGCTGGAGC |
| qPCR | | [SEQ ID NO: 13] |
| mmMPC1 | REV | GCAACAGAGGGCGAAAGTCATCCG |
| qPCR | | [SEQ ID NO: 14] |
| mmMPC2 | FWD | CCGCTTTACAACCACCCGGCA |
| qPCR | | [SEQ ID NO: 15] |
| mmMPC2 | REV | CAGCACACACCAATCCCCATTTCA |
| qPCR | | [SEQ ID NO: 16] |

Statistical Analysis

Quantitative results were analyzed for statistically significant effects. P-values determined by Student's t test using Microsoft Excel. All quantitative data are reported as mean±SEM. Significance is denoted as ***P<0.001, **P<0.01, and *P<0.05.

Mass Spectrometric Identification of Mpc1 and Mpc2 Interacting Proteins

Eluates from TAP purification of Mpc1-His6/HA2 were subjected to trypsin digestion and mass spectrometric protein identification. Proteins that were absent from a negative control purification and are annotated as mitochondrial are shown, along with the Peptide-Spectral Matches (PSMs). Size of the protein is also shown as this correlates with number of possible tryptic peptides that can be detected.

TABLE 2

| | | |
|---|---|---|
| Protein | PSMs | Protein Length (aa) |

Mpc1 TAP purification

| | | |
|---|---|---|
| Yg1080 (Mpc1) | 9 | 139 |
| Mpm1 | 7 | 252 |
| Gut2 | 7 | 649 |
| Ygr243 (Mpc3) | 5 | 146 |
| Fmp24 | 4 | 501 |
| Yhr162 (Mpc2) | 4 | 129 |

Mpc2 TAP purification

| | | |
|---|---|---|
| Md12 | 28 | 773 |
| Yhr162 (Mpc2) | 24 | 129 |
| Yg1080 (Mpc1) | 17 | 130 |
| Nde1 | 6 | 560 |
| Cor1 | 5 | 457 |
| Ygr243 (Mpc3) | 5 | 146 |
| Gut2 | 4 | 649 |
| Qcr2 | 3 | 368 |

### I. REFERENCES

1. D. Hanahan, et al., Cell 144, 646-74 (2011).
2. S. E. Kahn, et al., Nature 444, 840-6 (2006).
3. A. P. Halestrap, et al., Biochem J 138, 313-6 (1974).
4. A. P. Halestrap, Biochem J 172, 377-87 (1978).
5. A. P. Halestrap, Biochem J 148, 85-96 (1975).
6. S. Todisco, et al., J Biol Chem 281, 1524-31 (2006).
7. J. C. Hildyard, et al., Biochem J 374, 607-11 (2003).
8. M. Jiang, et al., Molecular Biology Reports 36, 215-20 (2009).
9. D. J. Pagliarini, et al., Cell 134, 112-23 (2008).
10. A. Sickmann, et al., Proc Natl Acad Sci USA 100, 13207-12 (2003).
11. H. Y. Steensma, et al., Eur J Biochem 191, 769-74 (1990).
12. E. Boles, et al., J Bacteriol 180, 2875-82 (1998).
13. S. Papa, et al., Eur J Biochem 49, 265-74 (1974).
14. M. Brivet, et al., Mol Genet Metab 78, 186-92 (2003).
15. A. P. Thomas, et al., Biochem J 196, 471-9 (1981).
16. R. A. Harris, et al., Adv Enzyme Regul 42, 249-59 (2002).
17. F. M. Zwiebel, et al., Biochemistry 21, 346-53 (1982).
18. R. Rognstad, Int J Biochem 15, 1417-21 (1983).
19. I. R. Boldogh, et al., Methods Cell Biol 80, 45-64 (2007).
20. I. Wittig, et al., Nat Protoc 1, 418-28 (2006).
21. J. Park et al., Nature 441, 1157-61 (2006).
22. L. Palanker, et al., Cell Metab 9, 228-39 (2009).
23. J. M. Tennessen, et al., Cell Metab 13, 139-48 (2011).
24. A. B. Canelas, et al., Anal Chem 81, 7379-89 (2009).
25. R. C. Cadwell, et al., PCR Methods Appl 3, S136-40 (1994).
26. L. M. Hinman, et al., J Biol Chem 256, 6583-6 (1981).
27. M. Brivet, et al., Mol Genet Metab 78, 186-92 (2003).
28. C. Stoetzel, et al., Am J Hum Genet 80, 1-11 (2007).
29. D. F. Gudbjartsson, et al., Nat Genet 25, 12-3 (2000).

1. (canceled)

2. A method of detecting an aberrant pyruvate metabolism associated condition in a subject comprising determining the expression level of MPC1 or MPC2 genes in a sample from the subject and comparing the expression level to the expression level of MPC1 or MPC2 genes in a normal sample.

3. The method of claim 2, wherein a decrease in the expression level of the MPC1 or MPC2 genes below the expression level in the normal sample indicates the presence of an aberrant pyruvate metabolism associated condition.

4. The method of claim 1, further comprising determining the activity of MPC1 or MPC2 in a sample from the subject and comparing the activity level to the activity level of MPC1 or MPC2 in a normal sample.

5. The method of claim 4, wherein a decrease in the activity of MPC1 or MPC2 below the activity level in the normal sample indicates the presence of an aberrant pyruvate metabolism associated condition.

6-8. (canceled)

9. A method for treating a subject with an aberrant pyruvate metabolism associated condition, the method comprising administering to the subject in need thereof an effective amount of one or more MPC1/MPC2 complex modulators as described herein in an amount sufficient to ameliorate the aberrant pyruvate metabolism associated condition.

10. The method of claim 9, wherein the one or more MPC1/MPC2 complex modulators comprises MPC1-D118G.

11. (canceled)

12. The method of claim 9, wherein the aberrant pyruvate metabolism associated conditions is diabetes, cancer, obesity, heart disease or cardiomyopathy.

13-43. (canceled)

44. The method of claim 9, wherein the one or more MPC1/MPC2 complex modulators restore MPC1 gene expression or MPC1 protein activity in the subject.

45. A method of screening for a pharmaceutical agent effective in treating a mitochondrial pyruvate oxidation defect comprising administering one or more pharmaceutical agents to a subject, determining MPC1 gene expression levels and/or MPC1 protein activity levels in the subject, and comparing those expression or activity levels to the MPC1 gene expression or MPC1 protein activity levels prior to administering the pharmaceutical agent, wherein an increase in the MPC1 gene expression levels or MPC1 protein activity levels in the subject after administration indicates efficacy of the pharmaceutical agent.

46. The method of claim 45, wherein the pharmaceutical agent is an orphan drug.

47-51. (canceled)

52. The method of claim 45, wherein the pharmaceutical agent is a mutant MPC peptide.

53. The method of claim 45, wherein the pharmaceutical agent is an MPC1 inhibitor

54. The method of claim 45, wherein the pharmaceutical agent is an MPC modulator

55. The method of claim 45, wherein the pharmaceutical agent is an MPC1, MPC2, or MPC1/MPC2 complex modulator

56. The method of claim 45, wherein the pharmaceutical agent is a modified allosteric MPC inhibitors.
